(19) United States
(12) Patent Application Publication
(10) Pub. No.: US 2026/0184739 A1
(43) Pub. Date: Jul. 2, 2026

(54) METHOD OF PURIFYING HEMOPEXIN USING MIXED-MODE CHROMATOGRAPHY

(71) Applicant: CSL Behring AG, Bern (CH)

(72) Inventors: Karl Beaumont MCCANN, Bern (CH); Hal BRALEY, Bern (CH); Chathuri WIJEKOON, Bern (CH)

(73) Assignee: CSL Behring AG, Bern (CH)

(21) Appl. No.: 19/130,479

(22) PCT Filed: Dec. 22, 2023
(86) PCT No.: PCT/IB2023/063164

§ 371 Date: May 15, 2025

(30) Foreign Application Priority Data
Dec. 22, 2022 (AU) 2022903972

(51) Int. Cl. C07K 1/18; A61K 38/00; C07K 1/16; C07K 1/34; C07K 1/36; C07K 14/47

(52) U.S. Cl. CPC C07K 1/18; A61K 38/00; C07K 1/165; C07K 1/34; C07K 1/36; C07K 14/4717

(57) ABSTRACT

Disclosed herein is a method of purifying proteins, in particular hemopexin, comprising passing a solution comprising hemopexin and other proteins through a mixed-mode cation exchange chromatography resin under conditions that promote selective binding of hemopexin to the resin, and eluting the bound hemopexin from the resin. The present disclosure also extends to compositions comprising purified hemopexin and uses thereof.

### TECHNICAL FIELD

The present invention relates generally to a method of purifying proteins. More specifically, the present invention relates to a method of purifying hemopexin, and uses thereof.

### BACKGROUND

Haemolysis is characterized by the destruction of red blood cells and is a hallmark of anaemic disorders associated with red blood cell abnormalities, such as enzyme defects, haemoglobinopathies, hereditary spherocytosis, paroxysmal nocturnal haemoglobinuria and spur cell anaemia, as well as extrinsic factors such as splenomegaly, autoimmune disorders (e.g., Hemolytic disease of the newborn), genetic disorders (e.g., Sickle-cell disease or G6PD deficiency), microangiopathic haemolysis, Gram-positive bacterial infection (e.g., Streptococcus, Enterococcus and Staphylococcus), parasite infection (e.g., Plasmodium), toxins and trauma (e.g., burns). Haemolysis is also a common disorder of blood transfusions, particularly massive blood transfusions and in patients using an extracorporeal cardio-pulmonary support.

The adverse effects seen in patients with conditions associated with haemolysis are largely attributed to the release of iron and iron-containing compounds, such as haemoglobin (Hb) and heme, from red blood cells. Under physiological conditions, released haemoglobin is bound by soluble proteins, such as haptoglobin, and transported to macrophages and hepatocytes. However, where the incidence of haemolysis is accelerated and becomes pathological in nature, the buffering capacity of haptoglobin becomes overwhelmed. As a result, haemoglobin is quickly oxidised to ferri-haemoglobin, which in turn releases free heme (comprising protoporphyrin IX and iron). Whilst heme plays a critical role in several biological processes (e.g., as part of essential proteins such as haemoglobin and myoglobin), free heme is highly toxic. Free heme is a source of redox-active iron, which produces highly toxic reactive oxygen species (ROS) that damages lipid membranes, proteins and nucleic acids. Heme toxicity is further exacerbated by its ability to intercalate into lipid membranes, where is causes oxidation of membrane components and promotes cell lysis and death.

The evolutionary pressure of continuous low-level extracellular Hb/heme exposure has led to compensatory mechanisms that control the adverse effects of free Hb/heme under physiological steady-state conditions and during mild haemolysis. These systems include the release of a group of plasma proteins that bind Hb or heme, including the Hb scavenger protein, haptoglobin, and the heme scavenger proteins, hemopexin and α1-microglobin. However, while endogenous haptoglobin and hemopexin control the adverse effects of free Hb/heme under physiological steady-state conditions, they have little effect in maintaining steady-state Hb/heme levels under pathophysiological conditions, such as those associated with haemolysis.

Hemopexin preparations have been shown to exhibit serine protease activity (Lin et al., 2016, Molecular Medicine, 22:22-31), anti- and pro-inflammatory activity, inhibition of cellular adhesion and binding of certain divalent metal ions. Further, hemopexin infusion has been shown to alleviate heme-induced endothelial activation, inflammation and oxidative injury in animal models of haemolytic disorders, such as Sickle-cell disease and β-thalassemia. While purified hemopexin shows important therapeutic potential, the amount of hemopexin required to satisfy the expected market demand will require a high capacity purification process. However, previously developed processes for the purification of hemopexin from human plasma have been limited to small-scale production methods for research purposes only, e.g., toxicology studies and phase I clinical manufacture (see, e.g., WO 2014/055552; Tsutsui and Mueller, 1981, Analytical Biochemistry, 121:244-250; and Muller-Eberhard, 1988, Methods in Enzymology, 163:563-565).

There remains a need, therefore, to develop an improved method for the purification of hemopexin.

### SUMMARY OF THE INVENTION

In an aspect of the present invention, there is provided a method of purifying hemopexin from a solution containing hemopexin and other proteins, the method comprising:
(i) providing a solution comprising hemopexin and other proteins, wherein solution comprises less than about 300 mM NaCl;
(ii) passing the solution of step (i) through a mixed-mode cation exchange chromatography resin under conditions that promote selective binding of hemopexin to the resin over binding of the other proteins to the resin;
(iii) washing the resin after step (ii) to remove unbound proteins;
(iv) eluting the hemopexin bound to the resin after step (iii); and
(v) recovering the hemopexin eluted in step (iv).

In an embodiment, the method further comprises:
(vi) passing the recovered hemopexin eluate of step (v) through a mixed-mode anion exchange chromatography resin under conditions that allow any impurities in the recovered hemopexin eluate to bind to the resin while allowing the hemopexin to pass through the resin as an unbound fraction; and
(vii) recovering the unbound fraction comprising hemopexin.

In another aspect of the present invention, there is provided a composition comprising hemopexin recovered by the methods disclosed herein.

In another aspect of the present invention, there is provided a formulation comprising the composition disclosed herein and a pharmaceutically acceptable carrier.

In another aspect of the present invention, there is provided the composition or formulation disclosed herein for use as a medicament for treating a condition associated with haemolysis.

In another aspect of the present invention, there is provided a method of treating a condition associated with haemolysis, the method comprising administering to a subject in need thereof the composition or formulation disclosed herein.

In another aspect of the present invention, there is provided use of the composition or formulation disclosed herein in the manufacture of a medicament for treating a condition associated with haemolysis.

### BRIEF DESCRIPTION OF THE FIGURES

FIG. 1 shows that the mixed mode anion exchange chromatography resin Capto Adhere is able to separate hemopexin from other impurity proteins. A photographic representation of non-reducing SDS-PAGE of fractions eluted with a stepwise pH elution from mixed mode anion exchange chromatography screening using (A) Capto Adhere (Cytiva); (B) HEA Hypercel (Pall); (C) PPA Hypercel (Pall); and (D) MEP Hypercel (Pall). Lane 1 and 2=flow through, Lane 3=wash, Lane 4=pH 6, Lane 5=pH 5, Lane 6=pH 4, Lane 7=pH 3, Lane 8=blank, and Lane 9=control.

FIG. 2 shows the amount of protein in solution (%; y-axis) extracted from Fraction IV-4 paste across a pH range of pH 4 to pH 8 (pH; x-axis). Line indicated by t=transferrin, line indicated by a=albumin, line indicated by h=haptoglobin, and line indicated by H=hemopexin.

FIG. 3 shows the consistency of FIV-4 paste extraction across batches. A graphical representation of hemopexin concentration (g/L; y-axis) and FIV-4 paste batch. Hemopexin concentration as measured by reversed phase HPLC.

FIG. 4 shows that yield of hemopexin from FIV-4 paste can be increased by use of an extraction buffer with higher pH. A graphical representation of hemopexin concentration (g/L; y-axis) and FIV-4 paste batch (x-axis) following extraction at pH 6.2 (black bars) and 7.5 (grey bars).

FIG. 5 shows the development and optimisation of Capto MMC chromatography as an effective capture step for the commercial purification of hemopexin. A series of photographic representations of non-reduced SDS-PAGE analysis of fractions from Capto MMC chromatography of clarified FIV-4 extracted paste, loaded at (A) pH 5.0, (B) pH 6.0, and (C) pH 7.0, eluted with a stepwise NaCl gradient. Lane 1=protein marker, Lane 2=load, Lane 3=drop through, Lane 4=wash, Lane 5=50 mM NaCl, Lane 6=150 mM, Lane 7=300 mM NaCl, Lane 8=500 mM NaCl, Lane 9=1 M NaCl, and Lane 9=hemopexin control.

FIG. 6 shows the optimisation of loading and elution conditions of Capto MMC chromatography. A photographic representation of a non-reduced SDS-PAGE analysis of fractions from Capto MMC chromatography loaded at pH 6.5, 200 mM NaCl. Lane 1=protein marker (MW standards), Lane 2=hemopexin standard, Lane 3=extract, Lane 4=drop through, Lane 5=wash, pH 6.5, 200 mM NaCl, Lane 6=wash, pH 7.0, Lane 7=elution, pH 7.5, 150 mM NaCl, Lane 8=elution, pH 7.5, 500 mM NaCl, Lane 9=elution, pH 7.5, 1 M NaCl.

FIG. 7 shows the concentration of hemopexin in the unbound fraction (g/L; y-axis) from a Capto MMC chromatography column with differing loading amounts (g/L resin; x-axis).

FIG. 8 shows the purity of hemopexin product obtained through the three step chromatographic process (Capto MMC, Capto Adhere and Eshmuno CPS) following different load conditions at the Capto MMC step. (A) A photographic representation of non-reduced SDS-PAGE analysis of Eshmuno CPS eluate with hemopexin product obtained from differing Capto MMC loading conditions. (B) A graphical representation of hemopexin purity (%; y-axis) measured by RP-HPLC at various process stages, with Capto MMC loaded under different conditions. Circles represent Capto MMC eluate, triangles represent Capto Adhere eluate, squares represent Eshmuno CPS eluate.

FIG. 9 is a representative chromatogram of Capto MMC mixed-mode cation exchange chromatography, showing eluted protein concentration as UV absorbance (AU; y-axis) and time (minutes; x-axis). The hemopexin containing peak is labelled.

FIG. 10 shows the development and optimisation of conditions for further purification of hemopexin by Capto Adhere chromatography. Recovery of various proteins (%; y-axis) from the unbound fraction of a Capto Adhere mixed-mode anion exchange chromatographic column loaded with Capto MMC eluate under various pH and NaCl conditions (x-axis). Bars indicated by t=transferrin, bars indicated by a=albumin, bars indicated by h=haptoglobin, and bars indicated by H=hemopexin.

FIG. 11 shows the development and optimisation of conditions for further purification of hemopexin by Capto Adhere chromatography. Recovery (%; y-axis) of (A) hemopexin and (B) transferrin from Capto MMC eluate applied to a Capto Adhere mixed-mode anion exchange chromatographic column loaded under various pH and NaCl conditions (x-axis).

FIG. 12 shows the purity of hemopexin obtained from Capto Adhere mixed-mode anion exchange chromatography of Capto MMC eluate. A photographic representation of non-reducing SDS-PAGE analysis of unbound fractions from Capto MMC purified hemopexin loaded at pH 7.5 and various NaCl concentrations.

FIG. 13 shows the optimisation of hemopexin load limit of Capto Adhere mixed-mode anion exchange chromatographic column. A photographic representation of non-reducing SDS-PAGE analysis of unbound fractions from Capto Adhere chromatography. Lane labels indicate the amount of protein loaded per mL of resin.

FIG. 14 shows the robustness of Capto Adhere loading conditions. (A) A photographic representation of non-reducing SDS-PAGE analysis of unbound fractions from Capto Adhere loaded under various pH and NaCl conditions. Lane 1=MMC eluate feed material; Lane 2=pH 7.0, 100 mM NaCl; Lane 3=pH 7.0, 200 mM NaCl; Lane 4=pH 7.2, 150 mM NaCl; Lane 5=pH 7.5, 150 mM NaCl; Lane 6=pH 7.8, 150 mM NaCl; Lane 7=pH 8.0, 100 mM NaCl; Lane 8=pH 8.0, 200 mM NaCl; and Lane 9=pH 7.5, 150 mM NaCl. (B) A graphical representation of transferrin recovery (%, y-axis) from eluates from Capto Adhere loaded under various pH and NaCl conditions (x-axis) as measured by nephelometry or RP-HPLC.

FIG. 15 is a representative chromatogram from the Capto Adhere mixed-mode anion exchange chromatographic column showing protein recovery as UV absorbance (AU; y-axis) and time (minutes; x-axis). Hemopexin is contained in the unbound fraction.

FIG. 16 shows the viral inactivation kinetics during solvent detergent incubation of Capto MMC eluate. A graphical representation of titre (log₁₀TCID₅₀/mL; y-axis) and time (minutes; x-axis) is shown.

FIG. 17 shows the purification of hemopexin by Eshmuno CPS chromatography. A photographic representation of non-reduced SDS-PAGE analysis of fractions of SD treated Capto Adhere eluate, under various loading conditions. Lane 1=Mark 12 MW standard; Lane 2=thawed Capto MMC eluate; Lane 3=Capto Adhere unbound; Lane 4=Eshmuno CPS feed; Lane 5=Eshmuno CPS unbound; Lane 6=Eshmuno CPS 100 mM NaCl eluate; Lane 7=Eshmuno CPS 200 mM NaCl eluate; Lane 8=pH 8.0, 200 mM NaCl eluate; and Lane 9=pH 7.5, 150 mM NaCl eluate.

FIG. 18 shows the binding capacity of Eshmuno CPS resin. A graphical representation of the amount of hemopexin in the unbound fraction (mg/mL; y-axis) from a 5 mL Eshmuno CPS chromatography column with differing load amounts (mg/mL resin; x-axis).

FIG. 19 shows the robustness of loading conditions for Eshmuno CPS chromatography. (A) A graphical representation of hemopexin and transferrin recovery (%; y-axis) under various pH and conductivity conditions (x-axis) as calculated by immunonephelometric quantitation. (B) A photographic representation of non-reduced SDS-PAGE analysis of eluates from Eshmuno CPS chromatography column with differing pH and conductivity conditions. Lane 1=molecular weight (MW) marker; Lane 2=pH 5.8, 8 mS/cm; Lane 3=pH 5.8, 10 mS/cm; Lane 4=pH 5.8, 12 mS/cm; Lane 5=pH 6.0, 8 mS/cm; Lane 6=pH 6.0, 10 mS/cm; Lane 7=pH 6.0, 12 mS/cm; Lane 8=pH 6.2, 8 mS/cm; and Lane 9=pH 6.2, 10 mS/cm.

FIG. 20 shows the effect of pH and conductivity on virus filtration. A graphical representation of filter throughput (L/m²; y-axis) and time (min; x-axis) at a conductivity of 18 mS/cm (1), 18 mS/cm (2), 37 mS/cm and 54 mS/cm.

FIG. 21 shows the effect of the pre-filter to virus filter area ratio on virus filtration. (A) A graphical representation of filter throughput (L/m²; y-axis) and time (min; x-axis) with a pre-filter to virus filter surface area ratio of 0.75:1 and 0.22:1 using the Sartopore 2 XLM pre-filter. (B) A graphical representation of filter throughput (L/m²; y-axis) and time (min; x-axis) with a pre-filter to virus filter surface area ratio of 0.25:1 and 1.6:1 using a Virosart Max pre-filter.

FIG. 22 shows the effectiveness of the Asahi BioEX filtration step for removal of Minute Virus of Mice (MVM) from purified hemopexin. (A) A graphical representation of filter throughput (L/m²; y-axis) and time (min; x-axis). (B) Viral titres of purified hemopexin spiked with MVM, when filtered using an Asahi BioEx filter.

FIG. 23 shows the partitioning of heme-hemopexin complex and hemopexin over a Capto MMC chromatographic column. A graphical representation of absorbance (mAu; y-axis) and volume (mL; x-axis) of a 1:1 mixture of hemopexin and heme-hemopexin complex during Capto MMC chromatography.

FIG. 24 is a flow diagram of the hemopexin purification process in an embodiment disclosed herein.

FIG. 25 shows a series of photographic representations of non-reducing SDS-PAGE analysis of fractions from purification of hemopexin from several batches of Fraction V paste (A), (B) and (C), at small laboratory scale.

FIG. 26 shows hemopexin recovery and purity following the streamlined laboratory scale batch of the hemopexin purification process in an embodiment disclosed herein. (A) A graphical representation of step recovery (%; y-axis) across each chromatography step of the streamlined laboratory scale batch (x-axis). (B) A photographic representation of non-reduced SDS-PAGE analysis for process intermediates.

FIG. 27 shows the characterisation of hemopexin drug substance. A photographic representation of reducing SDS-PAGE analysis of hemopexin drug substance (DS) produced in two pilot scale batches (1) and (2). Gel is a compilation of two original gels.

FIG. 28 is a flow diagram of the pre-viral inactivation process steps of a hemopexin purification process in accordance with an embodiment disclosed herein. The process intermediates are shown in bold text.

FIG. 29 is a flow diagram of the post-viral inactivation process steps of a hemopexin purification process in accordance with an embodiment disclosed herein. The process intermediates are shown in bold text.

FIG. 30 is a flow diagram of the pre- and post-viral inactivation process steps of a hemopexin purification process in accordance with an embodiment disclosed herein.

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

It must be noted that, as used in the subject specification, the singular forms “a”, “an” and “the” include plural aspects unless the context clearly dictates otherwise. Thus, for example, reference to “a resin” includes a single resin, as well as two or more resins; reference to “the composition” includes a single composition, as well as two or more compositions; and so forth.

In the absence of any indication to the contrary, reference made to a “%” content throughout this specification is to be taken as meaning % w/w (weight/weight). For example, a solution comprising a hemopexin content of at least 80% of total protein is taken to mean a composition comprising a hemopexin content of at least 80% w/w of total protein.

As used herein, the “about”, as applied to one or more values, refer to a value that is similar to a stated reference value. In certain embodiments, the term “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In a particular embodiment, the term “about” means+10% of the recited value.

The present invention is predicated, at least in part, on the finding that hemopexin can be purified from human plasma at a commercial scale. Thus, in an aspect of the present invention, there is provided a method of purifying hemopexin from a solution containing hemopexin and other proteins, the method comprising:
(i) providing a solution comprising hemopexin and other proteins, wherein the solution comprises less than about 300 mM NaCl;
(ii) passing the solution of step (i) through a mixed-mode cation exchange chromatography resin under conditions that promote selective binding of hemopexin to the resin over binding of the other proteins to the resin;
(iii) washing the resin after step (ii) to remove unbound proteins;
(iv) eluting the hemopexin bound to the resin after step (iii); and
(v) recovering the hemopexin eluted in step (iv).

Hemopexin (Hx) has been described as a 60-kD plasma β-1B-glycoprotein comprising a single 439 amino acid long peptide chain, which forms two domains joined by an interdomain linker. It has the highest known affinity for heme (K_{d}<1 μM) of any characterized heme-binding protein and binds heme in an equimolar ratio between the two domains of Hx in a pocket formed by the interdomain linker.

Hemopexin represents the primary line of defence against heme toxicity attributed at least in part to its ability to bind heme with high affinity and function as a heme-specific carrier from the bloodstream to the liver. Hemopexin has also been reported to possess serine protease activity and several other functions, such as anti- and pro-inflammatory activities, the ability to inhibit cellular adhesion and the binding of certain divalent metal ions.

### Extraction and Clarification

Any suitable material comprising hemopexin can be used to prepare a solution for use in accordance with the methods described herein. Suitable material would be known to persons skilled in the art, illustrative examples of which include plasma fractions such as various supernatants and precipitates derived from plasma fractionation processes. In these processes, plasma is typically sequentially subjected to various physical purification methods (e.g., precipitation, filtration, and adsorption), leading to intermediate products enriched in certain proteins. The separation of individual plasma proteins by fractionation processes can be achieved by exploiting the fact that different plasma proteins have different solubilities depending on, for example, pH, temperature, and ionic strength, as well as different adsorption properties on different types of solid supports (for example). Suitable industrial scale plasma fractionation processes will be known to persons skilled in the art, illustrative examples of which include precipitation with cold ethanol, following protocols such as the Cohn/Oncley fractionation process or Kistler/Nitschmann fractionation process. Typical fractionation processes are reviewed in Schultze and Heremans (Molecular Biology of human proteins. Volume I: Nature and Metabolism of Extracellular Proteins (Elsevier Publishing Company 1966), p. 236-317). Illustrative examples of ethanol fractionation processes, including Cohn fractionation and Kistler-Nitschmann fractionation, are described, for example, by Cohn et al (J Am Chem Soc. 1946; 68:459-75), Kistler and Nitschmanns (1962. Vox Sang 7:414-424), Friedli and Morgenthaler (Lancet, 1985; 1 (8439): 1215) and Gregori et al (Biologicals, 2004; 32:1-10), the entire contants of which are incorporated herein by reference. Illustrative examples of suitable fractions include those derived from a Cohn Fraction or a Kistler-Nitschmann Fraction, or similar, obtained from cold-ethanol fractionation of blood derived plasma. Also contemplated are fractions obtained from plasma fractionation processes that do not include ethanol, illustrative examples of which include affinity purification (e.g., affinity chromatograph or immunoaffinity) and others described in Burnouf T (Transfus. Med. Rev. 2007; 21 (2): 101-117, the entire contents of which is incorporated herein by reference. In some embodiments, the hemopexin-comprising sample is selected from the group consisting of plasma, cryo-poor plasma, IgG depleted plasma, or a Cohn Fraction or Kistler-Nitchmann Fraction or similar obtained from cold-ethanol fractionation of blood derived plasma. In embodiments, the hemopexin-comprising sample is selected from the group comprising cryosupernatant, 8% ethanol Supernatant I, Suspension A, Supernatant II+III, Supernatant (I)+II+III, Supernatant II, Fraction III, Fraction IV (e.g., Fraction IV1 or Fraction IV4 supernatant or precipitate), Fraction V, Supernatant V, Supernatant A, Precipitate C, and other similar variant fractions and precipitates. Plasma fractions that are derived from an immunoglobulin purification process are also contemplated herein. Persons skilled in the art will understand that the solution comprising hemopexin may comprise other proteins, such as haptoglobin, transferrin and heme-hemopexin complexes. In some embodiments, where proteins such as haptoglobin, transferrin and/or heme-hemopexin complexes are present in the solution comprising hemopexin, it may be desirable to remove these, such as by chromatographic separation, prior to performing the methods described herein.

In an embodiment, the solution comprising hemopexin is a human plasma fraction.

As described elsewhere herein, the methods disclosed herein may be used for commercial/industrial scale purification of hemopexin. When using plasma fractions as a starting material, employing the methods described herein on a commercial/industrial scale may suitably involve the use of a plasma fraction derived from at least about 500 kg of plasma. Thus, in an embodiment, the plasma fraction is derived from at least about 500 kg of plasma, preferably from at least about 5,000 kg, preferably from at least about 7,500 kg, preferably from at least about 10,000 kg or preferably from at least about 15,000 kg of plasma. In another embodiment, employing the methods described herein on a commercial/industrial scale may suitably involve using a batch of Fraction IV-4 paste from 21,000 kg of plasma and, optionally, pooling a plurality of batches (2 or more, 3 or more, 4 or more, and so on) into a single batch of starting material.

The skilled person will understand that plasma for fractionation is the liquid component of blood remaining after separation of the cellular material from blood collected by suitable means known to persons skilled in the art, illustrative examples of which include continuous filtration or apheresis.

In an embodiment, the solution comprising hemopexin is derived from a Cohn Fraction or an equivalent fraction from another plasma fractionation process. In an embodiment, the solution comprising hemopexin is a Cohn Fraction IV supernatant, Cohn Fraction IV precipitate, or an equivalent from another plasma fractionation process. In an embodiment, the solution comprising hemopexin is derived from a Fraction IV₄ Precipitate.

Where the solution comprising hemopexin is derived from a precipitate (e.g., a Fraction IV₄ Precipitate), the precipitate may suitably be stored prior to purifying hemopexin in accordance with the methods disclosed herein. Suitable storage conditions would be known to persons skilled in the art, illustrative examples of which include freezing the precipitate comprising hemopexin before re-solubilisation at −20° C., −80° C. or using liquid nitrogen. Accordingly, in some embodiments, the precipitate comprising hemopexin is a frozen Cohn Fraction IV. In a particularly preferred embodiment the solution comprising hemopexin is derived from a frozen Fraction IV₄ Precipitate.

Persons skilled in the art will appreciate that frozen precipitate comprising hemopexin are to be thawed prior to performing the methods disclosed herein. Thawing of such frozen precipitate may be performed at any temperature, preferably a temperature in the range of from about 2 to about 30° C. (e.g., 2° C., 3° C., 4° C., 5° C., 6° C., 7° C., 8° C. and so on), more preferably at an ambient temperature.

It will be understood that, where the starting material is provided as a precipitate (e.g., Fraction IV₄ Precipitate), it will be necessary to solubilise or resuspend the precipitate to provide a suitable starting solution comprising hemopexin for the methods described herein. In an embodiment, the solution comprising hemopexin is prepared by (a) resuspending a starting material comprising hemopexin in an extraction buffer to obtain a solution of resuspended or solubilised hemopexin, (b) passing the resuspended hemopexin solution of step (a) through a filter, and (c) recovering from step (b) the solution comprising hemopexin. In an embodiment, the starting material comprising hemopexin is a Cohn Fraction IV. In an embodiment, the Cohn Fraction IV is a Cohn Fraction IV₄. In an embodiment, the Cohn Fraction IV is a Cohn Fraction IV₄ precipitate.

The extraction buffer used to resuspend the starting material comprising hemopexin can comprise any suitable agent or combination of agents that is capable of solubilising or resuspending hemopexin present in the starting material, while also providing a matrix that is compatible with, for example, clarification and further downstream purification of hemopexin.

In an embodiment, the extraction buffer comprises from about 20 mM to about 500 mM NaCl (e.g., about 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM or 500 mM NaCl).

Accordingly, in an embodiment, the extraction buffer comprises from about 20 mM to about 500 mM NaCl, preferably about 20 mM, preferably about 30 mM, preferably about 40 mM, preferably about 50 mM, preferably about 60 mM, preferably about 70 mM, preferably about 80 mM, preferably about 90 mM, preferably about 100 mM, preferably about 110 mM, preferably about 120 mM, preferably about 130 mM, preferably about 140 mM, preferably about 150 mM, preferably about 160 mM, preferably about 170 mM, preferably about 180 mM, preferably about 190 mM, preferably about 200 mM, preferably about 210 mM, preferably about 220 mM, preferably about 230 mM, preferably about 240 mM, preferably about 250 mM, preferably about 260 mM, preferably about 270 mM, preferably about 280 mM, preferably about 290 mM, preferably about 300 mM, preferably about 310 mM, preferably about 320 mM, preferably about 330 mM, preferably about 340 mM, preferably about 350 mM, preferably about 360 mM, preferably about 370 mM, preferably about 380 mM, preferably about 390 mM, preferably about 400 mM, preferably about 410 mM, preferably about 420 mM, preferably about 430 mM, preferably about 440 mM, preferably about 450 mM, preferably about 460 mM, preferably about 470 mM, preferably about 480 mM, preferably about 490 mM or preferably about 500 mM NaCl.

In an embodiment, the extraction buffer comprises about 400 mM NaCl.

In an embodiment, the extraction buffer comprises from about 20 mM to about 60 mM buffering agent. Suitable buffering agents will be familiar to persons skilled in the art, illustrative examples of which include sodium phosphate. In an embodiment, the extraction buffer comprises from about 20 mM to about 60 mM (e.g., about 20 mM, 30 mM, 40 mM, 50 mM, 60 mM) sodium phosphate. In an embodiment, the extraction buffer comprises from about 30 mM to about 50 mM sodium phosphate. In an embodiment, the extraction buffer comprises from about 20 mM sodium phosphate. In an embodiment, the extraction buffer comprises from about 30 mM sodium phosphate. In an embodiment, the extraction buffer comprises from about 40 mM sodium phosphate. In an embodiment, the extraction buffer comprises from about 50 mM sodium phosphate. In an embodiment, the extraction buffer comprises from about 60 mM sodium phosphate.

In an embodiment, the extraction buffer comprises about 40 mM sodium phosphate (Na₂HPO₄/NaH₂PO₄) and about 400 mM NaCl.

In an embodiment, the extraction buffer has a pH of from about 6 to about 8 (e.g., about 6, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0).

Accordingly, in an embodiment, the extraction buffer has a pH of from about 6 to about 8, preferably about 6, preferably about 6.1, preferably about 6.2, preferably about 6.3, preferably about 6.4, preferably about 6.5, preferably about 6.6, preferably about 6.7, preferably about 6.8, preferably about 6.9, preferably about 7.0, preferably about 7.1, preferably about 7.2, preferably about 7.3, preferably about 7.4, preferably about 7.5, preferably about 7.6, preferably about 7.7, preferably about 7.8, preferably about 7.9 or preferably about 8.0.

In an embodiment, the extraction buffer has a pH of from about 6.2 to about 7.5. In another embodiment, the extraction buffer has a pH of about 7.5.

The conductivity of the extraction buffer may suitably be in the range of from about 30 mS/cm to about 45 mS/cm (e.g., about 30 mS/cm, about 31 mS/cm, about 32 mS/cm, about 33 mS/cm, about 34 mS/cm, about 35 mS/cm, about 36 mS/cm, about 37 mS/cm, about 38 mS/cm, about 39 mS/cm, about 40 mS/cm, about 41 mS/cm, about 42 mS/cm, about 43 mS/cm, about 44 mS/cm, about 45 mS/cm). Thus, in an embodiment, the conductivity of the extraction buffer is from about 30 mS/cm to about 45 mS/cm. In an embodiment, the conductivity of the extraction buffer is from about 31 mS/cm to about 44 mS/cm. In an embodiment, the conductivity of the extraction buffer is from about 32 mS/cm to about 43 mS/cm. In an embodiment, the conductivity of the extraction buffer is from about 33 mS/cm to about 42 mS/cm. In an embodiment, the conductivity of the extraction buffer is from about 35 mS/cm to about 41 mS/cm. In an embodiment, the conductivity of the extraction buffer is from about 35 mS/cm to about 40 mS/cm. In an embodiment, the conductivity of the extraction buffer is from about 35 mS/cm to about 39 mS/cm. In an embodiment, the extraction buffer has a conductivity of about 42 mS/cm. Suitable methods for determining (measuring) conductivity of a solution, including those described herein, will be familiar to persons skilled in the art, illustrative examples of which include using a Thermo Fisher Orion Star A212 conductivity meter. The conductivity of the extraction buffer may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity is measured at ambient temperature. In an embodiment, conductivity is measured at a temperature of from about 18° C. to about 25° C.

Resuspension of the hemopexin in the extraction buffer may suitably be achieved by mixing the material comprising hemopexin and the extraction buffer for a period of time and under conditions suitable to achieve resuspension (i.e., dissolution) of the hemopexin. In an embodiment, the material comprising hemopexin and the extraction buffer are mixed for a period of from about 10 minutes to about 240 minutes (e.g., 10, 20, 30, 40, 50, 60, 70, 80, 90, 100, 110, 120, 130, 140, 150, 160, 170, 180, 190, 200, 210, 220, 230 or 240 minutes). In another embodiment, the material comprising hemopexin and the extraction buffer are mixed for a period of ≥120 minutes, for example, from about 120 minutes to about 240 minutes (e.g., 120, 130, 140, 150, 160, 170, 180, 190, 200, 210, 220, 230 or 240 minutes minutes).

In an embodiment, the material comprising hemopexin and the extraction buffer are mixed for at least about 20 minutes. In a preferred embodiment, the material comprising hemopexin and the extraction buffer are mixed (i.e., stirred) for at least about 60 minutes.

Mixing of the material comprising hemopexin and the extraction buffer may be achieved using any method known to persons skilled in the art, illustrative examples of which include stirring, vortexing, shaking, rotating, rocking and any other suitable form of agitation. In an embodiment, the material comprising hemopexin and the extraction buffer are mixed by vortexing. In accordance with this embodiment, the material comprising hemopexin and the extraction buffer are vortexed to from about 5% to about 20% of liquid depth (e.g., about 5%, 10%, 15% or 20% of liquid depth). In a preferred embodiment, the material comprising hemopexin and the extraction buffer are vortexed to about 10% of liquid depth.

In an embodiment disclosed herein, the solution comprising hemopexin is prepared by a process comprising resuspending the material comprising hemopexin in an extraction buffer at a ratio of material comprising hemopexin: extraction buffer of from about 1:2 to about 1:20, preferably about 1:2, preferably about 1:2.5, preferably about 1:3, preferably about 1:3.5, preferably about 1:4, preferably about 1:4.5, preferably about 1:5. preferably about 1:5.5, preferably about 1:6, preferably about 1:6.5, preferably about 1:7, preferably about 1:7.5, preferably about 1:8, preferably about 1:8.5, preferably about 1:9, preferably about 1:9.5, preferably about 1:10, preferably about 1:10.5, preferably about 1:11, preferably about 1:11.5, preferably about 1:12, preferably about 1:12.5, preferably about 1:13, preferably about 1:13.5, preferably about 1:14, preferably about 1:14.5, preferably about 1:15, preferably about 1:15.5, preferably about 1:16, preferably about 1:16.5, preferably about 1:17, preferably about 1:17.5, preferably about 1:18, preferably about 1:18.5, preferably about 1:19, preferably about 1:19.5, preferably about 1:20 or preferably about 1:20.5.

In an embodiment, the solution comprising hemopexin is prepared by a process comprising resuspending the material comprising hemopexin in an extraction buffer at a ratio of material comprising hemopexin: extraction buffer of about 1:2.5. In an embodiment, the starting material comprising hemopexin is a Cohn Fraction IV. In an embodiment, the Cohn Fraction IV is a Cohn Fraction IV₄. In an embodiment, the Cohn Fraction IV is a Cohn Fraction IV₄ precipitate.

In an embodiment, the resuspended hemopexin solution is passed through a depth filter.

In an embodiment, the depth filter is a cellulose depth filter (e.g., 3M™ 90SP Zeta Plus™ Pall™ EK1P™, ErtelAlsop™ 953P™, Cytiva™ Stax™ depth filters, 3M™ 70CA Zeta Plus™ lenticular depth filters).

Optimisation of filtration throughput may be achieved by adjusting, for example, any one or more of the filter area, frame depth and flowthrough pressure. Persons skilled in the art will appreciate that any adjustment to the filtration parameters may alter the clarity, throughput and consistency of the clarified solution comprising hemopexin.

In an embodiment, the filtration throughput is about from about 50 to about 200 L/m². In an embodiment, the filtration throughput is about from about 100 L/m².

In an embodiment, the filter area is from about 0.5 to about 2 m²/9 kg of Cohn Fraction IV-4 precipitate. In an embodiment, the filter area is about 1 m²/9 kg of Cohn Fraction IV-4 precipitate.

In another embodiment, the filter area is less than about 1 m²/9 kg Cohn Fraction IV-4 precipitate (i.e., about 0.0312 m²/L Cohn Fraction IV extract). Persons skilled in the art will appreciate that where the filter area is reduced to less than about 1 m²/9 kg Cohn Fraction IV-4 precipitate, corresponding adjustments to the other filtration parameters may also be required to maintain throughput, e.g., increasing the frame depth/volume due to the amount of solids present.

In an embodiment, the frame depth is from about 1 cm to about 10 cm, preferably from about 2 cm to about 6 cm, more preferably from about 3 cm to about 5 cm. In an embodiment, the frame depth is 4 cm.

Further improvements to filtration throughput may be achieved by pre-coating (i.e., pre-flushing) the depth filter with a filter aid (e.g., Celpure C1000) under conditions and in an amount sufficient to evenly coat the filter. The depth filter may be washed prior to pre-coating using a suitable buffer, as necessary. In an embodiment, the depth filter comprises a filter aid. In some embodiments, the depth filter does not comprise a filter aid. In an embodiment, the buffer is the extraction buffer described elsewhere herein (e.g., 40 mM sodium phosphate, 400 mM NaCl, pH 7.2).

In an embodiment, the amount of filter aid is sufficient to provide a pre-coat of about 2 mm, e.g., about 0.625 kg/m² filter. In an embodiment, the flush volume to pre-coat with filter aid is about 1 press volume. The flow rate of the filter aid is set to provide an even coat of the filter and to prevent pooling of the filter aid in the bottom of the frames. In an embodiment, the flow rate is about 6.25 L/m²/min. In an embodiment, the filter aid is applied to the filter under pressure (e.g., 0.5, 0.6, 0.7, 0.8, 0.9 or 1 bar).

In an embodiment, step (b) comprises passing the resuspended hemopexin solution of step (a) through a filter at a flow rate and under pressure sufficient to prevent clogging of the filter and to preserve throughput and clarification. In an embodiment, the flow rate is 6.25 L/m²/min. In an embodiment, the filtration pressure is less than about 2 bar. In another embodiment, the filtration pressure is less than about 1.5 bar. In yet another embodiment, the filtration pressure is less than about 1 bar.

Following filtration, the depth filter may be washed to optimise for hemopexin recovery, i.e., a post-wash. Suitable post-wash solutions and conditions would be known to persons skilled in the art. In an embodiment, the post-wash solution is the equilibrium buffer described elsewhere herein (e.g., 40 mM sodium phosphate, 225 mM NaCl, pH 6.4). In an embodiment, the flush volume of the post-wash solution sufficient to maximise recovery of hemopexin from the filter is 2.5 press volumes. In an embodiment, the flush volume is about 3.0 press volumes.

In an embodiment, the pH of the solution comprising hemopexin is adjusted to a value of from about 6.2 to about 6.6 (e.g., 6.2, 6.3, 6.4, 6.5 or 6.6).

Accordingly, in an embodiment, the pH of the solution comprising hemopexin is adjusted to a value of from about 6.2 to about 6.6, preferably about 6.2, preferably about 6.3, preferably about 6.4, preferably about 6.5 or preferably about 6.6.

In an embodiment, the pH of the solution comprising hemopexin is adjusted to about 6.4 (i.e., 6.4±0.1).

The pH of the solution comprising hemopexin may be adjusted with any suitable acidic solution known to persons skilled in the art, illustrative examples of which include hydrochloric acid (HCl).

The conductivity of the solution comprising hemopexin may be suitably adjusted to a value of from about 24 mS/cm to about 30 mS/cm (e.g., 24 mS/cm, 25 mS/cm, 26 mS/cm, 27 mS/cm, 28 mS/cm, 29 mS/cm or 30 mS/cm). The conductivity of the solution comprising hemopexin may also be suitably adjusted to a value of from about 20 mS/cm to about 30 mS/cm (e.g., about 20 mS/cm, about 21 mS/cm, about 22 mS/cm, about 23 mS/cm, about 24 mS/cm, about 25 mS/cm, about 26 mS/cm, about 27 mS/cm, about 28 mS/cm, about 29 mS/cm or about 30 mS/cm).

Thus, in an embodiment, the conductivity of the solution comprising hemopexin is adjusted to a value of from about 24 mS/cm to about 30 mS/cm, preferably from about 26 mS/cm to about 28 mS/cm, preferably about 24 mS/cm, preferably about 25 mS/cm, preferably about 26 mS/cm, preferably about 27 mS/cm, preferably about 28 mS/cm, preferably about 29 mS/cm, or preferably about 30 mS/cm. In an embodiment, the conductivity of the solution comprising hemopexin is from about 26 mS/cm to about 28 mS/cm, preferably about 26 mS/cm, preferably about 27 mS/cm or preferably about 28 mS/cm. In an embodiment, the conductivity of the solution comprising hemopexin is about 27 mS/cm. In another embodiment, the conductivity of the solution comprising hemopexin is adjusted to a value of from about 20 mS/cm to about 30 mS/cm, preferably from about 20 mS/cm to about 29 mS/cm, preferably from about 20 mS/cm to about 28 mS/cm, preferably from about 20 mS/cm to about 27 mS/cm, preferably from about 20 mS/cm to about 26 mS/cm, preferably from about 20 mS/cm to about 25 mS/cm, preferably about 20 mS/cm, preferably from about 26 mS/cm to about 28 mS/cm, preferably about 21 mS/cm, preferably about 22 mS/cm, preferably about 23 mS/cm,

In another embodiment, the conductivity of the solution comprising hemopexin is from about 20 mS/cm to about 28 mS/cm. In another embodiment, the conductivity of the solution comprising hemopexin is from about 20 mS/cm to about 26 mS/cm. In an embodiment, the conductivity of the solution comprising hemopexin is from about 21 mS/cm to about 26 mS/cm. In an embodiment, the conductivity of the solution comprising hemopexin is from about 22 mS/cm to about 26 mS/cm. In an embodiment, the conductivity of the solution comprising hemopexin is from about 23 mS/cm to about 26 mS/cm. In an embodiment, the conductivity of the solution comprising hemopexin is from about 22 mS/cm to about 25 mS/cm. In an embodiment, the conductivity of the solution comprising hemopexin about 23 mS/cm. In an embodiment, the conductivity of the solution comprising hemopexin about 24 mS/cm. In an embodiment, the conductivity of the solution comprising hemopexin about 25 mS/cm. The conductivity of the solution may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity is measured at ambient temperature. In an embodiment, conductivity is measured at a temperature of from about 18° C. to about 25° C.

The solution comprising hemopexin can be stored for future use.

In an embodiment, the solution comprising hemopexin is prepared by further filtration of a Cohn Fraction IV extract. For example, a Fraction IV-4 (FIV-4) paste can be used as the starting material and resuspended in a solution comprising 40 mM sodium phosphate, 400 mM NaCl, pH 7.5+/−0.1 and at a ratio of 2.5 kg buffer per kg of paste. The resuspended FIV-4 paste is then be filtered through 3M 90SP zeta plus filter media in a filter press using, for example, a filter area of 1 m²/32 L of extracted paste solution, and a frame thickness of 4 cm. The filter may optionally be pre-coated with a filter aid, such as Celpure C1000.

In an embodiment, the solution comprising hemopexin is passed through a fine filter having a pore size of about 0.5 μm or less to obtain a clarified solution comprising hemopexin.

In an embodiment, the area of the fine filter is from about 10 cm²/L to about 50 cm²/L (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cm²/L). In another embodiment, the filter area of the fine filter is about 33 cm²/L depth filtrate.

In an embodiment, the filtration pressure is less than about 5 bar (e.g., 5, 4.5, 4, 3.5, 3, 2.5, 2, 1.5 or 1 bar). In another embodiment, the filtration pressure is <1.5 bar.

The clarified solution comprising hemopexin can be stored for future use. In an embodiment, the clarified solution comprising hemopexin can be stored at <23° C. for up to about 24 hours.

In another embodiment, the clarified solution comprising hemopexin can be stored at from about 2° C. to about 8° C. for up to 48 hours.

### Chromatographic Purification of Hemopexin

Purification of proteins by chromatography can be performed using either axial flow columns, such as those available from Cytiva, Sartorius and Bio-Rad, or using radial flow columns, such as those available from Proxcys. Chromatography can also be conducted using expanded bed technologies known to persons skilled in the art.

Most chromatographic processes employ a solid support, also referred to interchangeably herein as a resin or matrix. Suitable solid supports would be familiar to persons skilled in the art and the choice will depend on the type of product to be purified. Examples of suitable solid supports include inorganic carriers, such as glass and silica gel, organic, synthetic or naturally occurring carriers, such agarose, cellulose, dextran, polyamide, polyacrylamides, vinyl copolymers of bifunctional acrylates, and various hydroxylated monomers, and the like. Commercially available carriers are sold under the names of Eshmuno™, Nuvia™, Sephadex™, Sepharose™, Hypercel™, Capto™, Fractogel™, MacroPrep™, Unosphere™, GigaCap™, Trisacryl™, Ultrogel™ Dynospheres™, Macrosorb™ and XAD™ resins.

As described elsewhere herein, the inventors have unexpectedly found that mixed-mode cation exchange chromatographic resins can be used for the selective retention of hemopexin.

### Mixed-Mode Chromatography

Mixed-mode chromatography (MMC) is a chromatographic technique used for the separation of proteins on the basis of two or more forms of interaction between the stationary phase and the protein to be separated (e.g., hydrophobic, hydrophilic, ionic). For a given mixed-mode chromatographic column, the predominant separation interaction will depend on the properties of the solution and the mobile phase conditions.

Persons skilled in the art will understand that any mixed-mode cation exchange chromatographic resin can be used to purify hemopexin from the solution, as long as the hemopexin is capable of binding to the chromatographic resin while allowing some impurities in the solution to pass though the resin. Suitable resins would be known to persons skilled in the art. Examples of suitable mixed-mode cation exchange chromatography resins are those comprising a ligand with the structure of formula (I) or (II):

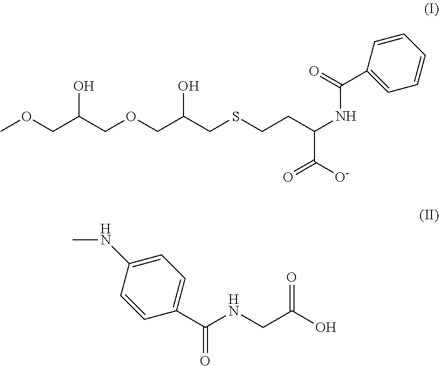 [SMILES: COCC(O)COCC(O)CSCCC(NC(=O)c1ccccc1)C(=O)[O-] ; CNc1ccc(C(=O)NCC(=O)O)cc1]

In an embodiment, the mixed-mode cation exchange chromatography resin comprises a ligand with the structure of formula I (e.g., Capto MMC™).

In an embodiment, the mixed-mode cation exchange chromatography resin comprises a ligand with the structure of formula II (e.g., Nuvia cPrime™).

The chromatography steps will generally be carried out under non-denaturing conditions and at convenient temperatures in the range of about 5° C. to +30° C., more usually at about ambient temperatures. The chromatographic steps may be performed batch-wise or continuously, as convenient.

Optimisation of chromatographic efficiency of the mixed-mode cation exchange chromatography resin may be achieved by adjusting variables such as pressure, temperature, column length, column bed height, height equivalent to a theoretical plate (HETP) and linear flow rate. Persons skilled in the art will appreciate that any adjustment to such variables may alter the selective binding of hemopexin and other proteins to the chromatographic column.

The chromatography column bed height will vary according to the specified product load and the column's pressure and performance range. In an embodiment, the column bed height is about 15 cm (i.e., 15±2 cm). For large-scale processes, the column bed height can be from about 10 cm to about 25 cm.

Persons skilled in the art will appreciate that the column linear flow rate should provide a convenient flow rate without the generation of significant backpressure. In an embodiment, the column linear flow rate is about 120 cm/hr.

Prior to loading the solution comprising hemopexin, an equilibration buffer may be applied to the mixed-mode chromatographic column to ensure that the pH and conductivity are equivalent to the solution comprising hemopexin (e.g., clarified Cohn Fraction IV extract). Suitable equilibration buffers would be known to persons skilled in the art, illustrative examples of which include the wash buffer described elsewhere herein (e.g., 40 mM sodium phosphate, 225 mM NaCl at pH 6.4). In an embodiment, the volume of the equilibration buffer required to pre-equilibrate is ≥1 column volumes (CV). In an embodiment, the pH of the mixed-mode chromatographic column following equilibration is from about pH 6.3 to about 6.5 (e.g., pH 6.3, 6.4 or 6.5). The conductivity of the equilibration buffer may suitably be from about 20 mS/cm to about 30 mS/cm (e. g., about 20 mS/cm, about 21 mS/cm, about 22 mS/cm, about 23 mS/cm, about 24 mS/cm, about 25 mS/cm, about 26 mS/cm, about 27 mS/cm, about 28 mS/cm, about 29 mS/cm or about 30 mS/cm). Thus, in an embodiment, the conductivity of the equilibration buffer is from about 20 mS/cm to about 30 mS/cm, preferably from about 21 mS/cm to about 29 mS/cm, preferably from about 22 mS/cm to about 28 mS/cm, preferably from about 23 mS/cm to about 28 mS/cm, preferably from about 23 mS/cm to about 27 mS/cm, preferably from about 23 mS/cm to about 26 mS/cm, or more preferably from about 23 mS/cm to about 25 mS/cm. In an embodiment, the conductivity of the equilibration buffer is from about 20 mS/cm to about 30 mS/cm. In an embodiment, the conductivity of the equilibration buffer is from about 20 mS/cm to about 30 mS/cm. In an embodiment, the conductivity of the equilibration buffer is from about 20 mS/cm to about 30 mS/cm. In an embodiment, the conductivity of the equilibration buffer is from about 23 mS/cm to about 28 mS/cm. In an embodiment, the conductivity of the equilibration buffer is from about 23 mS/cm to about 25 mS/cm. The conductivity of the equilibration buffer may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity is measured at ambient temperature. In an embodiment, conductivity is measured at a temperature of from about 18° C. to about 25° C.

The inventors have unexpectedly found that a solution comprising hemopexin and other proteins with a sodium chloride (NaCl) concentration less than about 300 nM (e.g., 10 mM, 20 mM, 30 mM, 40 mM, 50 mM, 60 mM, 70 mM, 80 mM, 90 mM, 100 mM, 210 mM, 220 mM, 230 mM, 240 mM, 250 mM, 260 mM, 270 mM, 280 mM, 290 mM or 300 mM) is optimal for loading the mixed-mode cation exchange chromatography resin to promote selective binding of hemopexin to the resin.

Accordingly, in an embodiment, the solution comprising hemopexin comprises less than about 300 mM NaCl, preferably about 10 mM, preferably about 20 mM, preferably about 30 mM, preferably about 40 mM, preferably about 50 mM, preferably about 60 mM, preferably about 70 mM, preferably about 80 mM, preferably about 90 mM, preferably about 100 mM, preferably about 210 mM, preferably about 220 mM, preferably about 230 mM, preferably about 240 mM, preferably about 250 mM, preferably about 260 mM, preferably about 270 mM, preferably about 280 mM, or preferably about 290 mM NaCl.

In an embodiment, the solution comprising hemopexin comprises from about 160 mM to about 250 mM NaCl. In another embodiment, the solution comprising hemopexin comprises from about 200 mM to about 250 mM NaCl. In a preferred embodiment, the solution comprising hemopexin comprises about 250 mM NaCl. In another embodiment, the solution comprising hemopexin comprises from about 220 mM to about 230 mM NaCl. In a preferred embodiment, the solution comprising hemopexin comprises about 225 mM NaCl.

The inventors have also shown a solution comprising hemopexin and other proteins with a pH maintained of less than about 8 (e.g., 7, 6, 5, 4 and values in between) is optimal for loading the mixed-mode cation exchange chromatography resin to promote selective binding of hemopexin to the resin.

Accordingly, in an embodiment, the solution comprising hemopexin has a pH of less than about 7, preferably about 6.5, preferably about 6, preferably about 5.5, preferably about 5, preferably about 4.5 or preferably about 4.

In an embodiment, the solution comprising hemopexin has a pH of from about 6.2 to about 6.6. In another embodiment, the solution of step (i) has a pH of about 6.4.

In an embodiment, the solution comprising hemopexin comprises:
(a) a pH of about 6.2 to about 6.6;
(b) from about 20 mM to about 60 mM phosphate buffer; and
(c) from about 160 mM to about 250 mM NaCl.

In another embodiment, the solution comprising hemopexin comprises:
(a) a pH of about 6.4;
(b) about 40 mM phosphate buffer; and
(c) about 225 mM NaCl.

In an embodiment, the amount of hemopexin that is passed through the resin in step (ii) is from about 1 mg to about 40 mg per mL of resin (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg per mL of resin).

Accordingly, in an embodiment, the amount of hemopexin that is passed through the resin in step (ii) is preferably about 1 mg, preferably about 2 mg, preferably about 3 mg, preferably about 4 mg, preferably about 5 mg, preferably about 6 mg, preferably about 7 mg, preferably about 8 mg, preferably about 9 mg, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg or preferably about 40 mg per mL of resin. In an embodiment, the amount of hemopexin that is passed through the resin in step (ii) is from about 10 mg/mL to about 20 mg/mL of resin. In an embodiment, the amount of hemopexin that is loaded onto the resin in step (ii) is from about 1 mg to about 40 mg per mL of resin (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg per mL of resin). In an embodiment, the amount of hemopexin that is loaded onto the resin in step (ii) is from about 10 mg/mL to about 20 mg/mL of resin.

Once the hemopexin is bound to the mixed-mode cation exchange chromatography resin, the resin may be washed to remove any residual impurities under conditions that retain the hemopexin bound to the resin. Suitable wash solutions and conditions will be known to persons skilled in the art. In an embodiment, the wash solution comprises 40 mM sodium phosphate, 225 mM NaCl at a pH of 6.4. In an embodiment, the volume of wash solution applied to the mixed-mode cation exchange chromatography resin is about 3 CV. The flow through wash fraction can also be collected and stored for future use, as necessary. The bound hemopexin can be eluted from the mixed-mode cation exchange chromatography resin by means known to persons skilled in the art.

Buffers that are suitable for eluting the hemopexin from the resin will also be known to persons skilled in the art, illustrative examples of which include phosphate. In an embodiment, the elution buffer comprises 40 mM sodium phosphate at a pH of about 7.5.

In an embodiment, the elution buffer further comprises from about 100 mM to about 200 mM NaCl. This equates to an elution buffer having a conductivity range of about 10 mS/cm (100 mM NaCl) to about 18 mS/cm (200 mM NaCl). In particular embodiments the elution buffer comprises about 140 to 160 mM NaCl. In an embodiment, the elution buffer comprises about 150 mM NaCl. However, persons skilled in the art would know that the NaCl concentration of the elution buffer may depend on the protein composition applied to the column and that adjustments beyond the described limits may be required to achieve the necessary recovery and purity of the hemopexin eluted from the resin.

In an embodiment, the elution buffer comprises from about 20 mM to about 60 mM sodium phosphate (e.g., about 20 mM, 30 mM, 40 mM, 50 mM, 60 mM sodium phosphate). In an embodiment, the extraction buffer comprises from about 30 mM to about 50 mM sodium phosphate. In an embodiment, the elution buffer comprises from about 20 mM sodium phosphate. In an embodiment, the elution buffer comprises from about 30 mM sodium phosphate. In an embodiment, the elution buffer comprises from about 40 mM sodium phosphate. In an embodiment, the elution buffer comprises from about 50 mM sodium phosphate. In an embodiment, the elution buffer comprises from about 60 mM sodium phosphate.

The conductivity of the elution buffer may suitably be from about 16 mS/cm to about 24 mS/cm (e. g., about 16 mS/cm, about 17 mS/cm, about 18 mS/cm, about 19 mS/cm, about 20 mS/cm, about 21 mS/cm, about 22 mS/cm, about 23 mS/cm, or about 24 mS/cm). Thus, in an embodiment, the conductivity of the elution buffer is from about 16 mS/cm to about 24 mS/cm, preferably from about 17 mS/cm to about 23 mS/cm, preferably from about 17 mS/cm to about 22 mS/cm, preferably from about 17 mS/cm to about 21 mS/cm, preferably from about 17 mS/cm to about 20 mS/cm, or more preferably from about 17 mS/cm to about 19 mS/cm. In an embodiment, the conductivity of the elution buffer is from about 17 mS/cm to about 21 mS/cm. In an embodiment, the conductivity of the elution buffer is from about 17 mS/cm to about 20 mS/cm. In an embodiment, the conductivity of the elution buffer is from about 17 mS/cm to about 19 mS/cm. The conductivity of the elution buffer may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity is measured at ambient temperature. In an embodiment, conductivity is measured at a temperature of from about 18° C. to about 25° C.

In some embodiments, it is advantageous that the volume of elution buffer applied to the mixed-mode cation exchange chromatography resin is sufficient to completely elute the bound hemopexin from the resin. However, in some instances, it may be sufficient to elute only a fraction of the bound hemopexin from the resin. In an embodiment the volume of the elution buffer applied to the mixed-mode cation exchange chromatography resin is about 3 CV.

In an embodiment, collection of the hemopexin eluate commences after about 0.5 column volumes (CV) of elution buffer application to the mixed-mode cation exchange chromatography resin and continues until the elution peak falls below A₂₈₀ₙₘ≤50 mAU (2 mm path length).

In an embodiment, the recovered hemopexin eluate will suitably have a purity (e.g., substantially purity) of at least about 50% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%).

Accordingly, in an embodiment, the recovered hemopexin eluate has a purity of preferably at least 50%, preferably at least 51%, preferably at least 52%, preferably at least 53%, preferably at least 54%, preferably at least 55%, preferably at least 56%, preferably at least 57%, preferably at least 58%, preferably at least 59%, preferably at least 60%, preferably at least 61%, preferably at least 62%, preferably at least 63%, preferably at least 64%, preferably at least 65%, preferably at least 66%, preferably at least 67%, preferably at least 68%, preferably at least 69%, preferably at least 70%, preferably at least 71%, preferably at least 72%, preferably at least 73%, preferably at least 74%, preferably at least 75%, preferably at least 76%, preferably at least 77%, preferably at least 78%, preferably at least 79%, preferably at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99%, or preferably 100%.

In an embodiment, the recovered hemopexin eluate has a purity of from about 70% to about 99%.

In an embodiment, the conductivity of the recovered hemopexin eluate is from about 16 mS/cm to about 22 mS/cm (e.g., about 16 mS/cm, about 17 mS/cm, about 18 mS/cm, about 19 mS/cm, about 20 mS/cm, about 21 mS/cm, or about 22 mS/cm). In an embodiment, the conductivity of the recovered hemopexin eluate is from about 17 mS/cm to about 21 mS/cm. In an embodiment, the conductivity of the recovered hemopexin eluate is from about 17 mS/cm to about 20 mS/cm. In an embodiment, the conductivity of the recovered hemopexin eluate is from about 17 mS/cm to about 19 mS/cm. In an embodiment, the conductivity of the recovered hemopexin eluate is about 17 mS/cm. In an embodiment, the conductivity of the recovered hemopexin eluate is about 18 mS/cm. In an embodiment, the conductivity of the recovered hemopexin eluate is about 19 mS/cm. In an embodiment, the conductivity of the recovered hemopexin eluate is about 20 mS/cm. In an embodiment, the conductivity of the recovered hemopexin eluate is from about 18 to about 19 mS/cm. The conductivity of the recovered hemopexin eluate may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the recovered hemopexin eluate is measured at ambient temperature. In an embodiment, conductivity of the recovered hemopexin eluate is measured at a temperature of from about 18° C. to about 25° C.

In an embodiment, the recovered hemopexin eluate can be stored for future use. In an embodiment, the recovered hemopexin eluate can be stored at a temperature of less than about 23° C. for up to 48 hours. In another embodiment, the recovered hemopexin eluate can be stored at a temperature of from about 2° C. to about 8° C. for at least 7 days.

In an embodiment, the recovered hemopexin eluate is further purified, e.g., by concentrating and diafiltering the eluted hemopexin through an ultrafiltration membrane, sterile filtering the concentrated and/or diafiltering hemopexin, and/or by further chromatographic purification, as required. In an embodiment, the recovered hemopexin eluate is further purified by ultrafiltration. In an embodiment, the recovered hemopexin eluate is further purified by tangential flow filtration. In another embodiment, the recovered hemopexin eluate is further purified by single-pass tangential flow filtration.

### Mixed-Mode Anion Exchange Chromatography

In an embodiment, the method described herein further comprises:
(vi) passing the recovered hemopexin eluate of step (v) through a mixed-mode anion exchange chromatography resin under conditions that allow any impurities in the recovered hemopexin eluate to bind to the resin while allowing the hemopexin to pass through the resin as an unbound fraction; and
(vii) recovering the unbound fraction comprising hemopexin.

Persons skilled in the art will understand that any mixed-mode anion exchange chromatographic resin can be used to further purify hemopexin from the recovered hemopexin eluate, as long as other proteins and impurities in the recovered hemopexin eluate are capable of binding to the chromatographic resin while suitably allowing hemopexin from the recovered hemopexin eluate to pass through the resin. By evaluating a number of different mixed-mode anion exchange chromatographic resins, the inventors have unexpectedly found mixed-mode anion exchange chromatographic resins comprising a N-benzyl methyl ethanolamine ligand (e.g., Capto Adhere™) is particularly suitable for further purification of hemopexin, including for commercial or industrial scale manufacture. Thus, in an embodiment, the mixed-mode anion exchange chromatography resin comprises a N-benzyl methyl ethanolamine ligand.

Optimisation of chromatographic efficiency of the mixed-mode anion exchange chromatographic resin may be achieved by adjusting variables such as temperature, column length, column bed height, height equivalent to a theoretical plate (HETP) and linear flow rate. Persons skilled in the art will appreciate that any adjustment to such variables may alter the binding of impurities to the chromatographic column or the elution of hemopexin in the unbound fraction.

In an embodiment, the column bed height is about 15 cm (i.e., 15±2 cm).

In an embodiment, the column linear flow rate is about 120 cm/hr.

Solutions that are suitable for the equilibration of the mixed-mode anion exchange resin (herein also referred to as an equilibration buffer) may comprise a buffering agent at a concentration of from about 10 mM to about 200 mM, preferably from about 10 to about 60 mM, or more preferably about 40 mM. The pH of the equilibration buffer may be in the range of 5 to about 9 and the conductivity of the equilibration buffer may suitably be less than about 18 mS/cm. In an embodiment, the conductivity of the equilibration buffer is from about 16 mS/cm to about 22 mS/cm (e.g., about 16 mS/cm, about 17 mS/cm, about 18 mS/cm, about 19 mS/cm, about 20 mS/cm, about 21 mS/cm, or about 22 mS/cm). In an embodiment, the conductivity of the equilibration buffer is about 17 mS/cm. In an embodiment, the conductivity of the equilibration buffer is about 18 mS/cm. In an embodiment, the conductivity of the equilibration buffer is about 19 mS/cm. In an embodiment, the conductivity of the equilibration buffer is about 20 mS/cm. The conductivity of the equilibration buffer may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the equilibration buffer is measured at ambient temperature. In an embodiment, conductivity of the equilibration buffer is measured at a temperature of from about 18° C. to about 25° C.

In an embodiment, the mixed-mode anion exchange chromatography resin is equilibrated with an equilibration buffer comprising a pH of about 7.0, preferably about 7.1, preferably about 7.2, preferably about 7.3, preferably about 7.4, preferably about 7.5, preferably about 7.6, preferably about 7.7, preferably about 7.8, preferably about 7.9 or preferably about 8.0.

In an embodiment, the equilibration buffer has a pH of about 7.5.

In an embodiment, the equilibration buffer comprises from about 100 mM to about 200 mM NaCl (e.g., 100 mM, 110 mM, 120 mM, 130 mM, 140 mM, 150 mM, 160 mM, 170 mM, 180 mM, 190 mM or 200 mM NaCl).

Accordingly, in an embodiment, the equilibration buffer comprises about 100 mM, preferably about 110 mM, preferably about 120 mM, preferably about 130 mM, preferably about 140 mM, preferably about 150 mM, preferably about 160 mM, preferably about 170 mM, preferably about 180 mM, preferably about 190 mM or preferably about 200 mM.

In another embodiment, the equilibration buffer comprises about 150 mM NaCl.

In an embodiment, the volume of the equilibrium buffer required to pre-equilibrate is ≥1 column volumes (CV). In an embodiment, the volume of the equilibrium buffer required to pre-equilibrate is ≥3 CV. In an embodiment, the volume of the equilibrium buffer required to pre-equilibrate is about 3 CV. In an embodiment, the pH of the effluent from the mixed-mode anion exchange chromatographic resin following pre-equilibration is from about pH 7.4 to about 7.6 (e.g., pH 7.4, 7.5 or 7.6). In an embodiment, the conductivity of the effluent from the mixed-mode anion exchange chromatographic resin following pre-equilibration is about 20 mS/cm.

Prior to step (vi), the concentration of the hemopexin in the recovered hemopexin eluate may be suitably concentrated, for example, by passing the recovered hemopexin eluate through an ultrafiltration membrane. Thus, in an embodiment, prior to step (vi), the concentration of the hemopexin in the recovered hemopexin eluate is concentrated. In an embodiment, the concentration of the hemopexin in the recovered hemopexin eluate is concentrated by passing the recovered hemopexin eluate through an ultrafiltration membrane. In an embodiment, the concentration of the hemopexin in the concentrated hemopexin eluate is from about 10 mg/mL to about 30 mg/mL (e.g., about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL), preferably from about 10 mg/mL to about 30 mg/mL, from about 15 mg/mL to about 25 mg/mL or more preferably about 20 mg/mL.

In an embodiment, the amount of hemopexin that is passed through the resin in step (vi) is from about 20 g to about 50 g per L of resin (e.g., about 20 g, about 21 g, about 22 g, about 23 g, about 24 g, about 25 g, about 26 g, about 27 g, about 28 g, about 29 g, about 30 g, about 31 g, about 32 g, about 33 g, about 34 g, about 35 g, about 36 g, about 37 g, about 38 g, about 39 g, about 40 g, about 41 g, about 42 g, about 43 g, about 44 g, about 45 g, about 46 g, about 47 g, about 48 g, about 49 g or about 50 g per L of resin). In another embodiment, the amount of hemopexin that is passed through the resin in step (iv) is about 30 g per L of resin.

The unbound fraction comprising hemopexin that is recovered in step (vii) may suitably have a conductivity of from about 16 mS/cm to about 22 mS/cm (e.g., about 16 mS/cm, about 17 mS/cm, about 18 mS/cm, about 19 mS/cm, about 20 mS/cm, about 21 mS/cm, or about 22 mS/cm). In an embodiment, the conductivity of the recovered unbound fraction is from about 17 mS/cm to about 21 mS/cm. In an embodiment, the conductivity of the recovered unbound fraction is from about 17 mS/cm to about 20 mS/cm. In an embodiment, the conductivity of the recovered unbound fraction is about 17 mS/cm. In an embodiment, the conductivity of the recovered unbound fraction is about 18 mS/cm. In an embodiment, the conductivity of the recovered unbound fraction is about 19 mS/cm. In an embodiment, the conductivity of the recovered unbound fraction is about 20 mS/cm. The conductivity of the recovered unbound fraction may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the recovered unbound fraction is measured at ambient temperature. In an embodiment, conductivity of the recovered unbound fraction is measured at a temperature of from about 18° C. to about 25° C.

After the recovered hemopexin eluate of step (v) has been passed through the resin in step (vi), the resin may be washed to ensure that all of the hemopexin is collected in the unbound hemopexin fraction. Suitable wash solutions and conditions will be known to persons skilled in the art. In an embodiment, the wash solution comprises 40 mM sodium phosphate, 150 mM NaCl at a pH of 7.5. In an embodiment, the volume of wash solution applied to the mixed-mode anion exchange chromatography resin is about 3 CV. In an embodiment, the conductivity of the wash solution is from about 16 mS/cm to about 22 mS/cm (e.g., about 16 mS/cm, about 17 mS/cm, about 18 mS/cm, about 19 mS/cm, about 20 mS/cm, about 21 mS/cm, or about 22 mS/cm). In an embodiment, the conductivity of the wash solution is about 17 mS/cm. In an embodiment, the conductivity of the wash solution is about 18 mS/cm. In an embodiment, the conductivity of the wash solution is about 19 mS/cm. In an embodiment, the conductivity of the wash solution is about 20 mS/cm. The conductivity of the wash solution may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the wash solution is measured at ambient temperature. In an embodiment, conductivity of the wash solution is measured at a temperature of from about 18° C. to about 25° C.

In an embodiment, collection of the unbound fraction comprising hemopexin commences at A₂₈₀ₙₘ≥50 mAU (when measured at the column outlet; 2 mm path length) and ends at A₂₈₀ₙₘ≤50 mAU (when measured at the column outlet; 2 mm path length).

In an embodiment, the unbound fraction comprising hemopexin can be stored for future use. In an embodiment, the unbound fraction comprising hemopexin can be stored at <23° C. for up to 48 hours. In another embodiment, the unbound fraction comprising hemopexin can be stored at from about 2° C. to about 8° C. for up to 7 days.

### Virus Inactivation

Where hemopexin is to be used for clinical or veterinary applications (e.g., for administration to a subject with a condition associated with haemolysis), it may be desirable to reduce the level of active virus content (i.e., virus titre) and other potential infectious agents (e.g., prions). This may be desirable where, for example, the feedstock comprising hemopexin and other proteins (i.e., the starting material/solution) is derived from blood plasma. Methods of reducing the virus titre in a solution will be known to persons skilled in the art. Examples include pasteurization (e.g., incubating the solution at 60° C. for 10 hours in the presence of high concentrations of stabilisers such as glycine (e.g., 2.75M) and sucrose (e.g., 50%) and/or other selected excipients or salts), dry heat treatment, virus filtration (e.g., passing the solution through a nano-filter, e.g., 20 nm cut-off) and/or subjecting the solution to treatment with a suitable organic solvent and surfactant for a period of time and under conditions to inactivate virus in the solution. Solvent detergent (SD) has been used for over 20 years to inactivate enveloped viruses particularly in plasma-derived products. Thus it may be carried out using various reagents and methods known in the art (see, e.g., U.S. Pat. Nos. 4,540,573 and 4,764,369, which are hereby incorporated by reference). Suitable solvents include tri-n-butyl phosphate (TnBP) and ether, preferably TnBP (typically at about 0.3%). Suitable detergents include polysorbate (Tween) 80, polysorbate (Tween) 20 and Triton X-100 (typically at about 0.3%). The selection of treatment conditions including solvent and detergent concentrations depend in part on the characteristics of the feedstock. Less pure feedstock generally require higher concentrations of reagents and more extreme reaction conditions. A preferred detergent is polysorbate 80 and a particularly preferred combination is polysorbate 80 and tri-n-butyl phosphate (TnBP). The feedstock may be stirred with solvent and detergent reagents at a temperature and for a time sufficient to inactivate any enveloped viruses that may be present. For example, the solvent detergent treatment may be carried out for about 2 to 24 hours at 23±2° C. The solvent detergent chemicals are subsequently removed by for example adsorption on chromatographic media such as C-18 hydrophobic resins or eluting them in the drop-through fraction of ion exchange resins under conditions which adsorb the protein of interest.

The virus inactivation step can be performed at any suitable stage of the methods disclosed herein. In an embodiment, the unbound fraction comprising hemopexin is subject to a viral inactivation step after step (vii). Prior to the virus inactivation step, the unbound fraction comprising hemopexin recovered in step (vii) may be suitably concentrated, for example, to minimise the volume of product handled during subsequent processing steps, including virus inactivation. Thus, in an embodiment, the method described herein further comprises concentrating the unbound fraction comprising hemopexin recovered in step (vii). In an embodiment, the unbound fraction comprising hemopexin recovered in step (vii) is concentrated prior to the virus inactivation step. Suitable methods for concentrating the unbound fraction comprising hemopexin recovered in step (vii) will be familiar to persons skilled in the art, illustrative examples of which include ultrafiltration/diafiltration using, for example, ultrafiltration/diafiltration membranes such as the Millipore Pellicon 3 cassette with Biomax (PES), Pellicon 2 cassette (Millipore) or Polyethersulfone or Hydrosart cassette (Sartorius). In an embodiment, the unbound fraction comprising hemopexin recovered in step (vii) is concentrated by ultrafiltration.

Employing a virus inactivation step, such as solvent detergent treatment after passing the partially purified hemopexin (i.e., the unbound fraction comprising hemopexin) through a mixed-mode anion exchange chromatographic resin, advantageously avoids solvent and detergent interference with purification and limits the number of purification steps required to be performed. This is particularly advantageous for industrial scale manufacture.

In an embodiment disclosed herein, the viral inactivation step comprises exposing the unbound fraction comprising hemopexin of step (vii) to a solution comprising a surfactant and a solvent.

In an embodiment, the temperature of the unbound fraction comprising hemopexin prior to the addition of a solution comprising a surfactant and a solvent is from about 21° C. to about 25° C. (e.g., 21° C., 22° C., 23° C., 24° C. or 25° C.).

In an embodiment, the solvent is tri-n-butyl phosphate (TnBP).

In an embodiment, the surfactant is polysorbate 80 (PS80).

In an embodiment, the solvent detergent treatment comprises exposing the recovered unbound fraction of step (vii) to 1% polysorbate 80 (PS80) and 0.3% tri-n-butyl phosphate (TnBP).

The unbound fraction comprising hemopexin and the solution comprising a surfactant and a solvent may be incubated for a time and under conditions suitable to achieve viral clearance. For example, the unbound fraction comprising hemopexin and the solution comprising a surfactant and a solvent may be mixing (i.e., stirring) using any method known to persons skilled in the art, illustrative examples of which include stirring, shaking, rotating and rocking. In an embodiment, the unbound fraction comprising hemopexin and the solution comprising a surfactant and a solvent are mixed by stirring. In accordance with this embodiment, the unbound fraction comprising hemopexin and the solution comprising a surfactant and a solvent are stirred such that a vortex occurs from about 5% to about 20% of liquid depth (e.g., about 5%, 10%, 15% or 20% of liquid depth). In an embodiment, the unbound fraction comprising hemopexin and the solution comprising a surfactant and a solvent are stirred with a vortex of about 5-10% of liquid depth.

In an embodiment, the unbound fraction comprising hemopexin and the solution comprising a surfactant and a solvent are incubated at about 23° C. (i.e., 23° C.±2° C.). In another embodiment, the unbound fraction comprising hemopexin and the solution comprising a surfactant and a solvent are incubated for from about 1 hour to about 24 hours (e.g., 1, 2, 3, 4, 5, 6, 7, 8, 9, 10, 11, 12, 13, 14, 15, 16, 17, 18, 19, 20, 21, 22, 23 or 24 hours).

Viral inactivation, including as described herein, may suitably further comprise adjusting the solution to a low pH. Low pH may be a pH of from about 2 to about 4. In an embodiment, low pH viral inactivation is performed in the presence of caprylate.

In another example, viral inactivation may be effected by contacting the fraction comprising hemopexin, with n-Octyl-β-D-Glucopyranoside (OG), thereby forming an OG-IgG mixture.

In a further example, low pH viral inactivation is performed in the presence of N,N-Dimethylmyristylamine N-oxide (TDAO).

In a further example, viral inactivation may be effected by exposing the fraction comprising hemopexin to a solvent-detergent inactivation step. Suitable solvent-detergent treatments would be known to the persons skilled in the art, illustrative examples of which include detergents, including biodegradable and/or environmentally friendly detergents. Exemplary biodegradable and/or environmentally friendly detergents suitable for use in a viral inactivation step, in particular for inactivating lipid enveloped viruses, include N,N-Dimethylmyristylamine N-oxide (TDAO), polysorbate 80 (PS80), polyoxyethylene (10) isooctylcyclohexyl ether (TRITON® X-100-reduced), and a non-ionic surfactant prepared from glucose and alcohol (e.g., Simulsol™ formulations). In an embodiment, the detergent is N,N-Dimethylmyristylamine N-oxide (TDAO). In another embodiment, the detergent is polysorbate 80. In another embodiment, the detergent is polyoxyethylene (10) isooctylcyclohexyl ether (TRITON® X-100-reduced). In yet another embodiment, the detergent is a non-ionic surfactant prepared from glucose and alcohol.

The methods disclosed herein may further comprise a virus filtration step. For example, virus filtration membranes of pore sizes of from about 15 nm to about 20 nm may be used to remove microbes and viruses from a solution. Illustrative examples of suitable nanofilters include Planova S20N (Asahi), Virosart HC (Sartorius) and Planova 20N (Asahi). In an embodiment, the recovered hemopexin is subjected to virus filtration. In an embodiment, the virus filtration comprises passing the recovered hemopexin through a virus filter having a pore size of from about 15 nm to about 20 nm diameter.

In an embodiment, the methods described herein further comprise ultrafiltration/diafiltration of any solution described herein comprising hemopexin. Illustrative examples of suitable ultrafiltration/diafiltration membranes include Pellicon 2 Cassettes (Millipore) or Polyethersulfone or Hydrosart cassettes (Sartorius).

### Ion Exchange Chromatography

In an embodiment, the method described herein further comprises:
(ix) passing the virus inactivated hemopexin solution through an ion exchange chromatography resin under conditions that allow the hemopexin to bind to the resin;
(x) optionally washing the resin following step (ix); and
(xi) eluting the hemopexin bound to the resin in step (ix); and
(xii) recovering the eluted hemopexin from step (xi).

Ion exchange chromatography is based on the attachment of amino acids (e.g., histidine) to positively or negatively charged functional groups, allowing proteins with a net negative charge (i.e., captured with a positively charged anion exchange resin) or a net positive charge (i.e., captured with a negatively charged cation exchange resin) to be retained in a column containing immobilized functional groups, such as —N+(C₂H₅)₂, —N+(CH₃)₃, —COO⁻ or —SO₃⁻.

In an embodiment, the ion exchange chromatography resin is a cation exchange chromatography resin (e.g., Eshmuno CPS) or an anion exchange chromatography resin (e.g., Capto Q™). In an embodiment, the ion exchange chromatography resin is a cation exchange chromatography resin. In an embodiment, the ion exchange chromatography resin is an anion exchange chromatography resin.

Optimisation of chromatographic efficiency of the ion exchange chromatography resin may be achieved by adjusting variables such as pressure, temperature, column length, column bed height, height equivalent to a theoretical plate (HETP) and linear flow rate. Persons skilled in the art will appreciate that any adjustment to such variables may alter the selective binding of hemopexin to the resin.

In an embodiment, the column bed height is from about 10 cm to about 25 cm, preferably at least about 10 cm, preferably at least about 11 cm, preferably at least about 12 cm, preferably at least about 13 cm, preferably at least about 14 cm, preferably at least about 15 cm, preferably at least about 16 cm, preferably at least about 17 cm, preferably at least about 18 cm, preferably at least about 19 cm, preferably at least about 20 cm, preferably at least about 21 cm, preferably at least about 22 cm, preferably at least about 23 cm, preferably at least about 24 cm, or more preferably about 25 cm. In an embodiment, the column bed height is about 15 cm (i.e., 15±2 cm).

In an embodiment, the column linear flow rate is from about 100 cm/hr to about 200 cm/hr, preferably from about 100 cm/hr to about 150 cm/hr, preferably from about 110 cm/hr to about 130 cm/hr, or more preferably from about 115 cm/hr to about 125 cm/hr. In an embodiment, the column linear flow rate is about 120 cm/hr.

The ion exchange chromatography resin may suitably be equilibrated in preparation for passage of the virus inactivated hemopexin solution in step (ix) described herein. This may suitably be achieved by passing through the ion exchange chromatography resin, before step (ix), a suitable equilibration buffer. The pH of the equilibration buffer may suitably be, preferably from about 5 to 7, more preferably about 6. In an embodiment, the equilibration buffer comprises from about 20 mM to about 60 mM buffering agent. Suitable buffering agents will be familiar to persons skilled in the art, illustrative examples of which include sodium phosphate. In an embodiment, the equilibration buffer comprises from about 20 mM to about 60 mM (e.g., about 20 mM, 30 mM, 40 mM, 50 mM, 60 mM) sodium phosphate. In an embodiment, the equilibration buffer comprises from about 30 mM to about 50 mM sodium phosphate. In an embodiment, equilibration buffer comprises from about 20 mM sodium phosphate. In an embodiment, the equilibration buffer comprises from about 30 mM sodium phosphate. In an embodiment, the equilibration buffer comprises from about 40 mM sodium phosphate. Solutions that are suitable for the equilibration of the pH (i.e., a pH equilibration buffer) of the ion exchange chromatography resin will normally be in the range of 5 to about 9. In an embodiment, the pH equilibration buffer comprises 40 nM sodium phosphate at a pH of 6.0. Additional solutions for the equilibration of the conductivity (i.e., an equilibration buffer) of the ion exchange chromatography resin are also contemplated herein and typically comprise a concentration of a buffering agent of about 20 mM to about 100 mM to obtain a conductivity of about 10 mS/cm. In an embodiment, the equilibration buffer comprises 25 mM sodium phosphate, 25 mM sodium acetate, 38 mM NaCl at a pH of 6.0. In an embodiment, the conductivity of the equilibration buffer for the ion exchange chromatography resin is from about 8 mS/cm to about 12 mS/cm, preferably about 8 mS/cm, preferably about 9 mS/cm, preferably about 10 mS/cm, preferably about 11 mS/cm or preferably about 12 mS/cm. In an embodiment, the conductivity of the equilibration buffer for the ion exchange chromatography resin from about 8 mS/cm to about 12 mS/cm (e.g., about 8 mS/cm, about 8.5 mS/cm, about 9 mS/cm, about 9.5 mS/cm, about 10 mS/cm, about 10.5 mS/cm, about 11 mS/cm, about 11.5 mS/cm, or about 12 mS/cm). In an embodiment, the conductivity of the equilibration buffer for the ion exchange chromatography resin from about 8 mS/cm to about 10 mS/cm. In an embodiment, the conductivity of the equilibration buffer for the ion exchange chromatography resin is from about 8.5 mS/cm to about 9.5 mS/cm. The conductivity of the equilibration buffer for the ion exchange chromatography resin may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the equilibration buffer for the ion exchange chromatography resin is measured at ambient temperature. In an embodiment, conductivity of the equilibration buffer for the ion exchange chromatography resin is measured at a temperature of from about 18° C. to about 25° C.

In an embodiment, the volume of the equilibrium buffer required to pre-equilibrate is ≥1 column volumes (CV). In an embodiment, the pH of the effluent from the ion exchange chromatography resin following pre-equilibration is from about pH 5.9 to about 6.1 (e.g., pH 5.9, 6.0 or 6.1). In an embodiment, the conductivity of the effluent from the mixed-mode anion exchange chromatographic resin following pre-equilibration is about 10 mS/cm.

In an embodiment, prior to step (ix), the pH of the virus inactivated hemopexin solution is adjusted to a value of from about pH 5.9 to about 6.2 (e.g., pH 5.9, 6.0, 6.1 or 6.2).

Accordingly, in an embodiment, prior to step (ix), the pH of the virus inactivated hemopexin solution is adjusted to a value of from about 5.9 to about 6.2, preferably about 5.9, preferably about 6.0, preferably about 6.1 or preferably about 6.2.

In an embodiment, prior to step (ix), the pH of the virus inactivated hemopexin solution is adjusted to about 6.0.

In an embodiment, prior to step (ix), the conductivity of the virus inactivated hemopexin solution is adjusted to a value of from about 8 mS/cm to about 12 mS/cm, preferably about 8 mS/cm, preferably about 9 mS/cm, preferably about 10 mS/cm, preferably about 11 mS/cm or preferably about 12 mS/cm. In an embodiment, the conductivity of the virus inactivated hemopexin solution is from about 8 mS/cm to about 12 mS/cm (e.g., about 8 mS/cm, about 8.5 mS/cm, about 9 mS/cm, about 9.5 mS/cm, about 10 mS/cm, about 10.5 mS/cm, about 11 mS/cm, about 11.5 mS/cm, or about 12 mS/cm). In an embodiment, the conductivity of the virus inactivated hemopexin solution is from about 8 mS/cm to about 10 mS/cm. In an embodiment, the conductivity of the virus inactivated hemopexin solution is from about 8.5 mS/cm to about 9.5 mS/cm. The conductivity of the virus inactivated hemopexin solution may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the virus inactivated hemopexin solution is measured at ambient temperature. In an embodiment, conductivity of the virus inactivated hemopexin solution is measured at a temperature of from about 18° C. to about 25° C.

In an embodiment, prior to step (ix), the conductivity of the virus inactivated hemopexin solution is adjusted to about 10 mS/cm (i.e., 10±2 mS/cm).

In an embodiment, the amount of hemopexin that is passed through the resin in step (ix) is from about 30 g to about 60 g per L of resin (e.g., about 30 g, about 31 g, about 32 g, about 33 g, about 34 g, about 35 g, about 36 g, about 37 g, about 38 g, about 39 g, about 40 g, about 41 g, about 42 g, about 43 g, about 44 g, about 45 g, about 46 g, about 47 g, about 48 g, about 49 g, about 50 g, about 51 g, about 52 g, about 53 g, about 54 g, about 55 g, about 56 g, about 57 g, about 58 g, about 59 g or about 60 g per L of resin). In another embodiment, the amount of hemopexin that is passed through the resin in step (ix) is about 40 g per L of resin.

Once the virus inactivated hemopexin solution is passed through the ion exchange chromatography resin, the flow through fraction can be collected and stored for future use.

The bound hemopexin can be eluted from the ion exchange chromatography resin by any means known to persons skilled in the art. Prior to eluting the hemopexin from the resin, the resin can be optionally washed with a suitable wash solution or buffer under conditions that retain the hemopexin bound to the resin. Suitable wash buffers and conditions would be known to persons skilled in the art. Wash buffer concentrations depend to a certain degree on column load, however, typical wash solutions possess a buffering effect at a pH of from about 6 to about 8. In an embodiment, the wash buffer comprises 25 mM sodium phosphate, 25 mM sodium acetate and 38 mM sodium chloride at a pH of 6.0. In an embodiment, the volume of wash solution applied to the mixed-mode anion exchange chromatography resin is about 3 CV. The flow through wash fraction can also be collected and stored for future use, as necessary.

In an embodiment, the bound hemopexin can be eluted from the ion exchange chromatography resin using an elution buffer comprising 20 mM sodium phosphate and 0.6 M sodium chloride at a pH of 7.2. In an embodiment, the volume of elution buffer applied to the ion exchange chromatography resin is about 3 CV. The elution buffer may suitably have a conductivity of from about 45 mS/cm to about 60 mS/cm (e.g., about 45 mS/cm, 46 mS/cm, 47 mS/cm, 48 mS/cm, 49 mS/cm, 50 mS/cm, 51 mS/cm, 52 mS/cm, 53 mS/cm, 54 mS/cm, 55 mS/cm, 56 mS/cm, 57 mS/cm, 58 mS/cm, 59 mS/cm, or 60 mS/cm). Thus, in an embodiment, the conductivity of the elution buffer is from about 45 mS/cm to about 60 mS/cm. In an embodiment, the conductivity of the elution buffer is from about 47 mS/cm to about 56 mS/cm. In an embodiment, the conductivity of the elution buffer is about 47 mS/cm. In an embodiment, the conductivity of the elution buffer is about 48 mS/cm. In an embodiment, the conductivity of the elution buffer is about 49 mS/cm. In an embodiment, the conductivity of the elution buffer is about 50 mS/cm. In an embodiment, the conductivity of the elution buffer is about 51 mS/cm. In an embodiment, the conductivity of the elution buffer is about 52 mS/cm. In an embodiment, the conductivity of the elution buffer is about 53 mS/cm. In an embodiment, the conductivity of the elution buffer is about 54 mS/cm. In an embodiment, the conductivity of the elution buffer is about 55 mS/cm. In an embodiment, the conductivity of the elution buffer is about 56 mS/cm. The conductivity of the elution buffer may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the elution buffer is measured at ambient temperature. In an embodiment, conductivity of the elution buffer is measured at a temperature of from about 18° C. to about 25° C.

In an embodiment, collection of the eluted hemopexin commences at A280 nm 50 mAU (2 mm path length) and ends at A₂₈₀ₙₘ≤50 mAU (when measured at the column outlet; 2 mm path length).

The eluted hemopexin that is recovered from the ion exchange chromatography resin may suitably have a conductivity of from about 35 mS/cm to about 50 mS/cm (e.g., about 35 mS/cm, 36 mS/cm, 37 mS/cm, 38 mS/cm, 39 mS/cm, 40 mS/cm, 41 mS/cm, 42 mS/cm, 43 mS/cm, 44 mS/cm, 45 mS/cm, 46 mS/cm, 47 mS/cm, 48 mS/cm, 49 mS/cm, or 50 mS/cm). Thus, in an embodiment, the conductivity of the eluted hemopexin is from about 35 mS/cm to about 50 mS/cm. In an embodiment, the conductivity of the eluted hemopexin is from about 35 mS/cm to about 45 mS/cm. In an embodiment, the conductivity of the eluted hemopexin is from about 40 mS/cm to about 45 mS/cm. In an embodiment, the conductivity of the eluted hemopexin is about 40 mS/cm. In an embodiment, the conductivity of the eluted hemopexin is about 41 mS/cm. In an embodiment, the conductivity of the eluted hemopexin is about 42 mS/cm. In an embodiment, the conductivity of the eluted hemopexin is about 43 mS/cm. In an embodiment, the conductivity of the eluted hemopexin is about 44 mS/cm. In an embodiment, the conductivity of the eluted hemopexin is about 45 mS/cm. The conductivity of the eluted hemopexin may be determined at any suitable temperature, preferably at ambient temperature, such as from about 18° C. to about 25° C., preferably about 18° C., or preferably about 19° C., or preferably about 20° C., or preferably about 21° C., or preferably about 22° C., or preferably about 23° C., or preferably about 24° C., or preferably about 25° C. In an embodiment, conductivity of the eluted hemopexin is measured at ambient temperature. In an embodiment, conductivity of the eluted hemopexin is measured at a temperature of from about 18° C. to about 25° C.

The eluted hemopexin that is recovered from the ion exchange chromatography resin can be stored for future use. In an embodiment, the eluted hemopexin can be stored at <23° C. for up to 24 hours. In another embodiment, the eluted hemopexin can be stored at from about 2° C. to about 8° C. for up to 7 days.

The eluted hemopexin may also be subjected to further purification, e.g., by concentrating and diafiltering the hemopexin through an ultramembrane and/or sterile filtering the concentrated and/or diafiltering hemopexin, as required. In some embodiments, the eluted hemopexin is subject to virus removal.

Techniques for virus removal based on size difference would be known to persons skilled in the art, illustrative examples of which include filtration and nanofiltration. In an embodiment, virus removal is performed by nanofiltration.

In an embodiment, virus removal is performed by pre-filtration in series with a virus filter. Suitable pre-filters would be known to persons skilled in the art, illustrative examples of which include nanofilters or other suitable filters having a pore size of about 0.1 μm or of about 0.2 μm (e.g., Sartopore 2XLM 0.1 μm). Other suitable filters would also be known to persons skilled in the art, illustrative examples of which include Planova BioEX and Virosart HF. In an embodiment, the virus filter has a pore size of less than about 0.2 μm, or preferably less than about 0.1 μm.

In an embodiment, the pre-filter area is sufficient to provide adequate pre-filtration with no product loss, e.g., >0.6 m²/100 L eluted hemopexin.

In an embodiment the virus filter area is sufficient to maintain throughput and achieve viral clearance, e.g., 1 m²/100 L eluted hemopexin.

In an embodiment, the pre-filter area is at least 0.6× the virus filter area.

In an embodiment, the pre-filter and virus filter are contacted with a wash solution prior to viral filtration of the eluted hemopexin. Suitable viral filtration wash solutions would be known to persons skilled in the art. In an embodiment, the virus filtration wash solution comprises NaCl and sodium phosphate. In an embodiment, the volume of virus filtration wash solution applied to the pre-filter and virus filter is at least about 6 capsule volumes. In an embodiment, the virus filtration wash solution is applied to the pre-filter and virus filter at a pressure selected from one or more or all of 0.3 bar, from about 0.5 to about 1 bar and about 3.0 bar (i.e., 3.0±0.1). Persons skilled in the art will appreciate that different pressures may be applied at different stages of filter preparation (i.e., pre-wash).

In an embodiment, the eluted hemopexin is applied to the pre-filter in series with a virus filter at a pressure of about 3.0 bar (i.e., 3.0±0.1).

Persons skilled in the art will appreciate that the protein load for application to the viral filters will be dependent on the filters used and the filter area. In an embodiment, the filter protein load is <1400 g/m².

In an embodiment, the pre-filter and virus filter are washed following filtration of the eluted hemopexin (i.e., post-wash). In an embodiment, the post-wash solution is the virus filtration solution described elsewhere herein. In an embodiment, the volume of virus filtration wash solution applied to the pre-filter and virus filter is at least about 3 capsule volumes. In an embodiment, the post-wash solution is applied to the pre-filter in series with a virus filter at a pressure of about 3.0 bar (i.e., 3.0±0.1).

The filtrate comprising hemopexin that is recovered from pre-filter and virus filter can be stored for future use. In an embodiment, the filtrate comprising hemopexin can be stored at <23° C. for up to 24 hours. In another embodiment, the filtrate comprising hemopexin can be stored at from about 2° C. to about 8° C. for up to 7 days.

### Concentration and Difiltration

In an embodiment, the method described herein further comprises exposing the eluted hemopexin recovered in step (xii) to ultrafiltration and/or diafiltration. In an embodiment, the eluted hemopexin recovered in step (xii) is exposed to ultrafiltration and/or diafiltration to adjust the concentration of the eluted hemopexin to a value of from about 50 mg/mL to about 120 mg/mL (e.g., about 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 111 mg/mL, 112 mg/mL, 113 mg/mL, 114 mg/mL, 115 mg/mL, 116 mg/mL, 117 mg/mL, 118 mg/mL, 119 mg/mL or 120 mg/mL).

Accordingly, in an embodiment, the concentration of the eluted hemopexin is adjusted to a value of from about 50 mg/mL to about 120 mg/mL, preferably about 50 mg/mL, preferably about 51 mg/mL, preferably about 52 mg/mL, preferably about 53 mg/mL, preferably about 54 mg/mL, preferably about 55 mg/mL, preferably about 56 mg/mL, preferably about 57 mg/mL, preferably about 58 mg/mL, preferably about 59 mg/mL, preferably about 60 mg/mL, preferably about 61 mg/mL, preferably about 62 mg/mL, preferably about 63 mg/mL, preferably about 64 mg/mL, preferably about 65 mg/mL, preferably about 66 mg/mL, preferably about 67 mg/mL, preferably about 68 mg/mL, preferably about 69 mg/mL, preferably about 70 mg/mL, preferably about 71 mg/mL, preferably about 72 mg/mL, preferably about 73 mg/mL, preferably about 74 mg/mL, preferably about 75 mg/mL, preferably about 76 mg/mL, preferably about 77 mg/mL, preferably about 78 mg/mL, preferably about 79 mg/mL, preferably about 80 mg/mL, preferably about 81 mg/mL, preferably about 82 mg/mL, preferably about 83 mg/mL, preferably about 84 mg/mL, preferably about 85 mg/mL, preferably about 86 mg/mL, preferably about 87 mg/mL, preferably about 88 mg/mL, preferably about 89 mg/mL, preferably about 90 mg/mL, preferably about 91 mg/mL, preferably about 92 mg/mL, preferably about 93 mg/mL, preferably about 94 mg/mL, preferably about 95 mg/mL, preferably about 96 mg/mL, preferably about 97 mg/mL, preferably about 98 mg/mL, preferably about 99 mg/mL, preferably about 100 mg/mL, preferably about 101 mg/mL, preferably about 102 mg/mL, preferably about 103 mg/mL, preferably about 104 mg/mL, preferably about 105 mg/mL, preferably about 106 mg/mL, preferably about 107 mg/mL, preferably about 108 mg/mL, preferably about 109 mg/mL, preferably about 110 mg/mL, preferably about 111 mg/mL, preferably about 112 mg/mL, preferably about 113 mg/mL, preferably about 114 mg/mL, preferably about 115 mg/mL, preferably about 116 mg/mL, preferably about 117 mg/mL, preferably about 118 mg/mL, preferably about 119 mg/mL or preferably about 120 mg/mL.

In an embodiment, the concentration of the eluted hemopexin is adjusted to about 100 mg/mL.

The methods described herein may suitably comprise adjusting or increasing the concentration of the hemopexin in the filtrate. Suitable methods of adjusting the concentration of the hemopexin in the filtrate will be familiar to persons skilled in the art, an illustrative example of which includes diafiltration.

In an embodiment, the concentration of hemopexin in the filtrate is adjusted by to a value of from about 50 mg/mL to about 120 mg/mL, preferably about 50 mg/mL, preferably about 51 mg/mL, preferably about 52 mg/mL, preferably about 53 mg/mL, preferably about 54 mg/mL, preferably about 55 mg/mL, preferably about 56 mg/mL, preferably about 57 mg/mL, preferably about 58 mg/mL, preferably about 59 mg/mL, preferably about 60 mg/mL, preferably about 61 mg/mL, preferably about 62 mg/mL, preferably about 63 mg/mL, preferably about 64 mg/mL, preferably about 65 mg/mL, preferably about 66 mg/mL, preferably about 67 mg/mL, preferably about 68 mg/mL, preferably about 69 mg/mL, preferably about 70 mg/mL, preferably about 71 mg/mL, preferably about 72 mg/mL, preferably about 73 mg/mL, preferably about 74 mg/mL, preferably about 75 mg/mL, preferably about 76 mg/mL, preferably about 77 mg/mL, preferably about 78 mg/mL, preferably about 79 mg/mL, preferably about 80 mg/mL, preferably about 81 mg/mL, preferably about 82 mg/mL, preferably about 83 mg/mL, preferably about 84 mg/mL, preferably about 85 mg/mL, preferably about 86 mg/mL, preferably about 87 mg/mL, preferably about 88 mg/mL, preferably about 89 mg/mL, preferably about 90 mg/mL, preferably about 91 mg/mL, preferably about 92 mg/mL, preferably about 93 mg/mL, preferably about 94 mg/mL, preferably about 95 mg/mL, preferably about 96 mg/mL, preferably about 97 mg/mL, preferably about 98 mg/mL, preferably about 99 mg/mL, preferably about 100 mg/mL, preferably about 101 mg/mL, preferably about 102 mg/mL, preferably about 103 mg/mL, preferably about 104 mg/mL, preferably about 105 mg/mL, preferably about 106 mg/mL, preferably about 107 mg/mL, preferably about 108 mg/mL, preferably about 109 mg/mL, preferably about 110 mg/mL, preferably about 111 mg/mL, preferably about 112 mg/mL, preferably about 113 mg/mL, preferably about 114 mg/mL, preferably about 115 mg/mL, preferably about 116 mg/mL, preferably about 117 mg/mL, preferably about 118 mg/mL, preferably about 119 mg/mL or preferably about 120 mg/mL

In an embodiment, the concentration of hemopexin in the filtrate is adjusted to about 100 mg/mL.

In another aspect disclosed herein, there is provided a method of purifying hemopexin from a solution containing hemopexin and other proteins, the method comprising:
(i) providing a solution comprising hemopexin and other proteins, wherein the solution comprises about 225 mM NaCl, about 40 mM sodium phosphate, a pH of about 6.4+/−1 and a conductivity of about 27+/−1 mS/cm;
(ii) passing the solution of step (i) through a mixed-mode cation exchange chromatography resin under conditions that promote selective binding of hemopexin to the resin over binding of the other proteins to the resin;
(iii) washing the resin after step (ii) to remove unbound proteins;
(iv) eluting the hemopexin bound to the resin after step (iii) with an elution buffer comprising about 150 mM NaCl, about 40 mM sodium phosphate and a pH of about 7.5+/−1;
(v) recovering the hemopexin eluted in step (iv);
(vi) passing the recovered hemopexin eluate of step (v) through a mixed-mode anion exchange chromatography resin under conditions that allow any impurities in the recovered hemopexin eluate to bind to the resin while allowing the hemopexin to pass through the resin as an unbound fraction;
(vii) recovering the unbound fraction comprising hemopexin; and
(viii) optionally, exposing the recovered unbound fraction of step (vii) to a virus inactivation step to obtain a virus inactivated hemopexin solution.

In another aspect disclosed herein, there is provided a method of purifying hemopexin from a solution containing hemopexin and other proteins, the method comprising:
(i) providing a solution comprising hemopexin and other proteins, wherein the solution comprises about 225 mM NaCl, about 40 mM sodium phosphate, a pH of about 6.4+/−1 and a conductivity of from about 23 to about 25 mS/cm;
(ii) passing the solution of step (i) through a mixed-mode cation exchange chromatography resin under conditions that promote selective binding of hemopexin to the resin over binding of the other proteins to the resin;
(iii) washing the resin after step (ii) to remove unbound proteins;
(iv) eluting the hemopexin bound to the resin after step (iii) with an elution buffer comprising about 150 mM NaCl, about 40 mM sodium phosphate, a pH of about 7.5+/−1 and a conductivity of from about 18 to about 21 mS/cm;
(v) recovering the hemopexin eluted in step (iv);
(vi) passing the recovered hemopexin eluate of step (v) through a mixed-mode anion exchange chromatography resin under conditions that allow any impurities in the recovered hemopexin eluate to bind to the resin while allowing the hemopexin to pass through the resin as an unbound fraction;
(vii) recovering the unbound fraction comprising hemopexin; and
(viii) concentrating the recovered unbound fraction from step (vii); and
(ix) optionally, exposing the concentrated unbound fraction of step (viii) to a virus inactivation step to obtain a virus inactivated hemopexin solution.

The methods described herein may suitably be performed batch-wise or continuously, as convenient.

### Compositions

In an aspect of the present invention, there is provided a composition comprising the hemopexin recovered by the methods disclosed herein. In an embodiment, the composition comprises a hemopexin content of at least 80% of total protein (e.g., at least 80%, 81%, 82%, 83%, 84%, 85%, 86%, 87%, 88%, 89%, 90%, 91%, 92%, 93%, 94%, 95%, 96%, 97%, 98%, 99%, or 100% of total protein).

In another embodiment, the composition comprises a hemopexin content of at least 90% of total protein. In another embodiment, the composition comprises a hemopexin content of at least 95%. In another embodiment, the composition comprises a hemopexin content of at least 97%. In yet another embodiment, the composition comprises a hemopexin content of at least 98%.

The composition comprising hemopexin recovered by the method of the present invention disclosed herein will be substantially free of other components with which they are normally associated (e.g., other plasma-derived proteins). Thus, in an embodiment, the composition comprising hemopexin will comprise less than 20% of total protein, preferably less than 10% of total protein, and more preferably less than 5% of total protein of other components with which they are normally associated (i.e., impurities). The skilled person will understand that the level of impurities present in the compositions of the present invention may depend on the intended use of the compositions. For example, where the compositions are to be administered to a human subject in need thereof (i.e., for clinical use), it would be desirable that the composition comprises less than 5% impurities (of total protein). Conversely, where the proteins are to be used in vitro, it may be acceptable if the composition comprises more than 5% of impurities (of total protein).

In another aspect disclosed herein, there is provided a formulation comprising the composition comprising hemopexin, as described herein, and a pharmaceutically acceptable carrier. Suitable pharmaceutically acceptable carriers, diluents and/or excipients are known to those skilled in the art, illustrative examples of which include solvents, dispersion media, antifungal and antibacterial agents, surfactants, isotonic and absorption agents and the like.

The formulation may also be formulated by the addition of (or a combination of) suitable stabilisers, for example, an amino acid, a carbohydrate, a salt, and a detergent. In particular embodiments, the stabiliser comprises a mixture of a sugar alcohol and an amino acid. The stabilizer may comprise a mixture of a sugar (e.g., sucrose or trehalose), a sugar alcohol (e.g., mannitol or sorbitol), and an amino acid (e.g., proline, glycine and arginine). In an embodiment, the formulation comprises an amino acid such as arginine. In other embodiments, the formulation comprises divalent metal ions in a concentration up to 100 mM and a complexing agent as described in U.S. Pat. No. 7,045,601. In embodiments where the pH is preferably about 6.5 to 7.5 and the osmolality is at least 240 mosmol/kg.

The formulation may also be sterilised by filtration prior to dispensing and long term storage. Preferably, the formulation will retain substantially its original stability characteristics for at least 2, 4, 6, 8, 10, 12, 18, 24, 36 or more months. For example, formulations stored at 2-8° C. or 25° C. can typically retain substantially the same molecular size distribution as measured by HPLC-SEC when stored for 6 months or longer. Particular embodiments of the pharmaceutical formulation can be stable and suitable for commercial pharmaceutical use for at least 6 months, 12 months, 18 months, 24 months, 36 months or even longer when stored at 2-8° C. and/or room temperature.

The compositions or formulations described herein may be formulated into any of many possible dosage forms such as injectable formulations. The formulations and their subsequent administration (dosing) are within the skill of those in the art. Dosing is dependent on the responsiveness of the subject to treatment, but will invariably last for as long as the desirable effect (e.g., a reduction in the level of free Hb/heme) is desired. Persons of ordinary skill can easily determine optimum dosages, dosing methodologies and repetition rates.

In an embodiment disclosed herein, the formulation has a volume of at least 5 mL and comprises at least 5 mg/mL hemopexin (e.g., 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 150 mg/mL, or 200 mg/mL). In another embodiment, the pharmaceutical formulation has a volume of at least 5 mL and comprises at least 20 mg/mL hemopexin. In particular embodiments, the formulation has a volume of at least 5 mL and comprises hemopexin at a concentration of about 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 90 mg/mL, 100 mg/mL, 150 mg/mL or 200 mg/mL. In another aspect, there is provided a vessel containing at least 5 mL of a formulation comprising hemopexin, wherein the concentration of hemopexin in the formulation is at least 20 mg/mL.

In a preferred embodiment, the formulation comprises about 15 mM citrate phosphate buffer, about 150 mM NaCl, wherein the pH is preferably about 7.2, and a concentration of hemopexin of about 100 mg/mL.

In an embodiment, the composition or formulation comprises a hemopexin content of from about 95 mg/mL to about 110 mg/mL. In an embodiment, the composition or formulation comprises a hemopexin content of from about 100 mg/mL to about 110 mg/mL. In an embodiment, the composition or formulation comprises a hemopexin content of from about 103 mg/mL to about 106 mg/mL. In an embodiment, the composition or formulation comprises a hemopexin content of from about 103 mg/mL to about 104 mg/mL. In an embodiment, the composition or formulation comprises a hemopexin content of from about 106 mg/mL to about 107 mg/mL. In an embodiment, the composition or formulation comprises a hemopexin content of about 100 mg/mL. In an embodiment, the composition or formulation comprises a hemopexin content of about 103 mg/mL. In an embodiment, the composition or formulation comprises a hemopexin content of about 106 mg/mL.

In an embodiment, the composition or formulation comprises heme binding activity of from about 1000 μM to about 2000 μM. In an embodiment, the composition or formulation comprises heme binding activity of from about 1300 μM to about 1900 μM. In an embodiment, the composition or formulation comprises heme binding activity of from about 1500 μM to about 1800 μM. In an embodiment, the composition or formulation comprises heme binding activity of from about 1600 μM to about 1800 μM. In an embodiment, the composition or formulation comprises a heme binding activity of about 1700 UM.

In an embodiment, the composition or formulation comprises heme specific binding activity of at least about 80% of total protein. In an embodiment, the composition or formulation comprises heme specific binding activity of at least about 85% of total protein. In an embodiment, the composition or formulation comprises heme specific binding activity of at least about 90% of total protein. In an embodiment, the composition or formulation comprises heme specific binding activity of at least about 91% of total protein. In an embodiment, the composition or formulation comprises heme specific binding activity of at least about 92% of total protein. In an embodiment, the composition or formulation comprises heme specific binding activity of at least about 93% of total protein. In an embodiment, the composition or formulation comprises heme specific binding activity of at least about 94% of total protein. In an embodiment, the composition or formulation comprises heme specific binding activity of at least about 95% of total protein. In an embodiment, the composition or formulation comprises heme specific binding activity of at least about 96% of total protein. In an embodiment, the composition or formulation comprises heme specific binding activity of at least about 97% of total protein. In an embodiment, the composition or formulation comprises heme specific binding activity of at least about 98% of total protein. In an embodiment, the composition or formulation comprises heme specific binding activity of at least about 99% of total protein. In an embodiment, the composition or formulation comprises heme specific binding activity of about 100% of total protein.

In an embodiment, the composition or formulation comprises a CD91 dissociation constant (KD) of from about 0.50 μM to about 2.0 μM. In an embodiment, the composition or formulation comprises a CD91 dissociation constant (KD) of from about 1.00 μM to about 1.5 μM. In an embodiment, the composition or formulation comprises a CD91 dissociation constant (KD) of from about 1.05 μM to about 1.15 μM. In an embodiment, the composition or formulation comprises a CD91 dissociation constant (KD) of from about 1.10 μM to about 1.20 μM. In an embodiment, the composition or formulation comprises a CD91 dissociation constant (KD) of about 1.13±0.06 μM. In an embodiment, the composition or formulation comprises a CD91 dissociation constant (KD) of about 1.06±0.05 M.

In an embodiment, the composition or formulation comprises a transferrin content of less than about 0.50 mg/mL. In an embodiment, the composition or formulation comprises a transferrin content of less than about 0.40 mg/mL. In an embodiment, the composition or formulation comprises a transferrin content of less than about 0.30 mg/mL. In an embodiment, the composition or formulation comprises a transferrin content of less than about 0.25 mg/mL. In an embodiment, the composition or formulation comprises a transferrin content of less than about 0.20 mg/mL. In an embodiment, the composition or formulation comprises a transferrin content of less than about 0.15 mg/mL. In an embodiment, the composition or formulation comprises a transferrin content of less than about 0.12 mg/mL. In an embodiment, the composition or formulation comprises a transferrin content of less than about 0.11 mg/mL.

In an embodiment, the composition or formulation comprises an albumin content of less than about 0.05 mg/mL. In an embodiment, the composition or formulation comprises an albumin content of less than about 0.025 mg/mL. In an embodiment, the composition or formulation comprises an albumin content of less than about 0.020 mg/mL. In an embodiment, the composition or formulation comprises an albumin content of less than about 0.015 mg/mL. In an embodiment, the composition or formulation comprises an albumin content of less than about 0.010 mg/mL. In an embodiment, the composition or formulation comprises an albumin content of less than about 0.009 mg/mL. In an embodiment, the composition or formulation comprises an albumin content of less than about 0.0085 mg/mL. In an embodiment, the composition or formulation comprises an albumin content of less than about 0.008 mg/mL.

In an embodiment, the composition or formulation comprises a haptoglobin content of less than about 0.05 mg/mL. In an embodiment, the composition or formulation comprises a haptoglobin content of less than about 0.04 mg/mL. In an embodiment, the composition or formulation comprises a haptoglobin content of less than about 0.03 mg/mL. In an embodiment, the composition or formulation comprises a haptoglobin content of less than about 0.025 mg/mL. In an embodiment, the composition or formulation comprises a haptoglobin content of less than about 0.02 mg/mL. In an embodiment, the composition or formulation comprises a haptoglobin content of less than about 0.015 mg/mL. In an embodiment, the composition or formulation comprises a haptoglobin content of less than about 0.010 mg/mL. In an embodiment, the composition or formulation comprises a haptoglobin content of less than about 0.005 mg/mL.

In an embodiment, the composition or formulation comprises an Apo-A1 content of less than about 0.10 mg/mL. In an embodiment, the composition or formulation comprises an Apo-A1 content of less than about 0.09 mg/mL. In an embodiment, the composition or formulation comprises an Apo-A1 content of less than about 0.08 mg/mL. In an embodiment, the composition or formulation comprises an Apo-A1 content of less than about 0.07 mg/mL. In an embodiment, the composition or formulation comprises an Apo-A1 content of less than about 0.06 mg/mL. In an embodiment, the composition or formulation comprises an Apo-A1 content of less than about 0.05 mg/mL.

In an embodiment, the composition or formulation comprises a high molecular weight (HMW) hemopexin aggregate content of less than about 1.0% of total protein. In an embodiment, the composition or formulation comprises a HMW hemopexin aggregate content of less than about 0.9% of total protein. In an embodiment, the composition or formulation comprises a HMW hemopexin aggregate content of less than about 0.8% of total protein. In an embodiment, the composition or formulation comprises a HMW hemopexin aggregate content of less than about 0.7% of total protein. In an embodiment, the composition or formulation comprises a HMW hemopexin aggregate content of less than about 0.6% of total protein. In an embodiment, the composition or formulation comprises a HMW hemopexin aggregate content of less than about 0.5% of total protein. In an embodiment, the composition or formulation comprises a HMW hemopexin aggregate content of less than about 0.4% of total protein. In an embodiment, the composition or formulation comprises a HMW hemopexin aggregate content of from about 0.4% to about 0.5% of total protein.

In an embodiment, the composition or formulation comprises a HMW hemopexin aggregate content of less than about 1.0% of total protein, as determined by size exclusion-high-performance liquid chromatography. In an embodiment, the composition or formulation comprises a HMW hemopexin aggregate content of less than about 0.9% of total protein, as determined by size exclusion-high-performance liquid chromatography. In an embodiment, the composition or formulation comprises a HMW hemopexin aggregate content of less than about 0.8% of total protein, as determined by size exclusion-high-performance liquid chromatography. In an embodiment, the composition or formulation comprises a HMW hemopexin aggregate content of less than about 0.7% of total protein, as determined by size exclusion-high-performance liquid chromatography. In an embodiment, the composition or formulation comprises a HMW hemopexin aggregate content of less than about 0.6% of total protein, as determined by size exclusion-high-performance liquid chromatography. In an embodiment, the composition or formulation comprises a HMW hemopexin aggregate content of less than about 0.5% of total protein, as determined by size exclusion-high-performance liquid chromatography. In an embodiment, the composition or formulation comprises a HMW hemopexin aggregate content of less than about 0.4% of total protein, as determined by size exclusion-high-performance liquid chromatography. In an embodiment, the composition or formulation comprises a HMW hemopexin aggregate content of from about 0.4% to about 0.5% of total protein, as determined by size exclusion-high-performance liquid chromatography.

In an embodiment, the composition or formulation comprises a hemopexin monomer content of at least about 90% of total protein. In an embodiment, the composition or formulation comprises a hemopexin monomer content of at least about 91% of total protein. In an embodiment, the composition or formulation comprises a hemopexin monomer content of at least about 92% of total protein. In an embodiment, the composition or formulation comprises a hemopexin monomer content of at least about 93% of total protein. In an embodiment, the composition or formulation comprises a hemopexin monomer content of at least about 94% of total protein. In an embodiment, the composition or formulation comprises a hemopexin monomer content of at least about 95% of total protein. In an embodiment, the composition or formulation comprises a hemopexin monomer content of at least about 96% of total protein. In an embodiment, the composition or formulation comprises a hemopexin monomer content of at least about 97% of total protein. In an embodiment, the composition or formulation comprises a hemopexin monomer content of at least about 98% of total protein. In an embodiment, the composition or formulation comprises a hemopexin monomer content of at least about 99% of total protein.

In an embodiment, the composition or formulation comprises a hemopexin monomer content of at least about 90% of total protein, as determined by size exclusion-high-performance liquid chromatography. In an embodiment, the composition or formulation comprises a hemopexin monomer content of at least about 91% of total protein, as determined by size exclusion-high-performance liquid chromatography. In an embodiment, the composition or formulation comprises a hemopexin monomer content of at least about 92% of total protein, as determined by size exclusion-high-performance liquid chromatography. In an embodiment, the composition or formulation comprises a hemopexin monomer content of at least about 93% of total protein, as determined by size exclusion-high-performance liquid chromatography. In an embodiment, the composition or formulation comprises a hemopexin monomer content of at least about 94% of total protein, as determined by size exclusion-high-performance liquid chromatography. In an embodiment, the composition or formulation comprises a hemopexin monomer content of at least about 95% of total protein, as determined by size exclusion-high-performance liquid chromatography. In an embodiment, the composition or formulation comprises a hemopexin monomer content of at least about 96% of total protein, as determined by size exclusion-high-performance liquid chromatography. In an embodiment, the composition or formulation comprises a hemopexin monomer content of at least about 97% of total protein, as determined by size exclusion-high-performance liquid chromatography. In an embodiment, the composition or formulation comprises a hemopexin monomer content of at least about 98% of total protein, as determined by size exclusion-high-performance liquid chromatography. In an embodiment, the composition or formulation comprises a hemopexin monomer content of at least about 99% of total protein, as determined by size exclusion-high-performance liquid chromatography.

In an embodiment, the composition or formulation comprises a low molecular weight (LMW) impurity content of less than about 1.0% of total protein. In an embodiment, the composition or formulation comprises an LMW impurity content of less than about 0.9% of total protein. In an embodiment, the composition or formulation comprises an LMW impurity content of less than about 0.8% of total protein. In an embodiment, the composition or formulation comprises an LMW impurity content of less than about 0.7% of total protein. In an embodiment, the composition or formulation comprises an LMW impurity content of less than about 0.6% of total protein. In an embodiment, the composition or formulation comprises an LMW impurity content of less than about 0.5% of total protein. In an embodiment, the composition or formulation comprises an LMW impurity content of less than about 0.4% of total protein. In an embodiment, the composition or formulation comprises an LMW impurity content of less than about 0.3% of total protein.

In an embodiment, the composition or formulation comprises a low molecular weight (LMW) impurity content of less than about 1.0% of total protein, as determined by size exclusion-high-performance liquid chromatography. In an embodiment, the composition or formulation comprises an LMW impurity content of less than about 0.9% of total protein, as determined by size exclusion-high-performance liquid chromatography. In an embodiment, the composition or formulation comprises an LMW impurity content of less than about 0.8% of total protein, as determined by size exclusion-high-performance liquid chromatography. In an embodiment, the composition or formulation comprises an LMW impurity content of less than about 0.7% of total protein, as determined by size exclusion-high-performance liquid chromatography. In an embodiment, the composition or formulation comprises an LMW impurity content of less than about 0.6% of total protein, as determined by size exclusion-high-performance liquid chromatography. In an embodiment, the composition or formulation comprises an LMW impurity content of less than about 0.5% of total protein, as determined by size exclusion-high-performance liquid chromatography. In an embodiment, the composition or formulation comprises an LMW impurity content of less than about 0.4% of total protein, as determined by size exclusion-high-performance liquid chromatography. In an embodiment, the composition or formulation comprises an LMW impurity content of less than about 0.3% of total protein, as determined by size exclusion-high-performance liquid chromatography.

In an embodiment, the composition or formulation comprises a hemopexin purity content of at least about 80% of total protein. In an embodiment, the composition or formulation comprises a hemopexin purity content of at least about 82% of total protein. In an embodiment, the composition or formulation comprises a hemopexin purity content of at least about 84% of total protein. In an embodiment, the composition or formulation comprises a hemopexin purity content of at least about 86% of total protein. In an embodiment, the composition or formulation comprises a hemopexin purity content of at least about 88% of total protein. In an embodiment, the composition or formulation comprises a hemopexin purity content of at least about 90% of total protein. In an embodiment, the composition or formulation comprises a hemopexin purity content of at least about 92% of total protein. In an embodiment, the composition or formulation comprises a hemopexin purity content of at least about 94% of total protein. In an embodiment, the composition or formulation comprises a hemopexin purity content of at least about 96% of total protein. In an embodiment, the composition or formulation comprises a hemopexin purity content of at least about 98% of total protein. In an embodiment, the composition or formulation comprises a hemopexin purity content of at least about 99% of total protein.

In an embodiment, the composition or formulation comprises a hemopexin purity content of at least about 80% of total protein, as determined by reduced SDS-PAGE. In an embodiment, the composition or formulation comprises a hemopexin purity content of at least about 82% of total protein, as determined by reduced SDS-PAGE. In an embodiment, the composition or formulation comprises a hemopexin purity content of at least about 84% of total protein, as determined by reduced SDS-PAGE. In an embodiment, the composition or formulation comprises a hemopexin purity content of at least about 86% of total protein, as determined by reduced SDS-PAGE. In an embodiment, the composition or formulation comprises a hemopexin purity content of at least about 88% of total protein, as determined by reduced SDS-PAGE. In an embodiment, the composition or formulation comprises a hemopexin purity content of at least about 90% of total protein, as determined by reduced SDS-PAGE. In an embodiment, the composition or formulation comprises a hemopexin purity content of at least about 92% of total protein, as determined by reduced SDS-PAGE. In an embodiment, the composition or formulation comprises a hemopexin purity content of at least about 94% of total protein, as determined by reduced SDS-PAGE. In an embodiment, the composition or formulation comprises a hemopexin purity content of at least about 96% of total protein, as determined by reduced SDS-PAGE. In an embodiment, the composition or formulation comprises a hemopexin purity content of at least about 98% of total protein, as determined by reduced SDS-PAGE. In an embodiment, the composition or formulation comprises a hemopexin purity content of at least about 99% of total protein, as determined by reduced SDS-PAGE.

In an embodiment, the composition or formulation comprises a hemopexin purity content of at least about 80% of total protein, as determined by non-reduced SDS-PAGE. In an embodiment, the composition or formulation comprises a hemopexin purity content of at least about 82% of total protein, as determined by non-reduced SDS-PAGE. In an embodiment, the composition or formulation comprises a hemopexin purity content of at least about 84% of total protein, as determined by non-reduced SDS-PAGE. In an embodiment, the composition or formulation comprises a hemopexin purity content of at least about 86% of total protein, as determined by non-reduced SDS-PAGE. In an embodiment, the composition or formulation comprises a hemopexin purity content of at least about 88% of total protein, as determined by non-reduced SDS-PAGE. In an embodiment, the composition or formulation comprises a hemopexin purity content of at least about 90% of total protein, as determined by non-reduced SDS-PAGE. In an embodiment, the composition or formulation comprises a hemopexin purity content of at least about 92% of total protein, as determined by non-reduced SDS-PAGE. In an embodiment, the composition or formulation comprises a hemopexin purity content of at least about 94% of total protein, as determined by non-reduced SDS-PAGE. In an embodiment, the composition or formulation comprises a hemopexin purity content of at least about 96% of total protein, as determined by non-reduced SDS-PAGE. In an embodiment, the composition or formulation comprises a hemopexin purity content of at least about 98% of total protein, as determined by non-reduced SDS-PAGE. In an embodiment, the composition or formulation comprises a hemopexin purity content of at least about 99% of total protein, as determined by non-reduced SDS-PAGE.

In an embodiment, the protein content of the composition or formulation comprises an isoelectric point (pI) of from about 5.0 to about 6.5. In an embodiment, the protein content of the composition or formulation comprises an isoelectric point (pI) of from about 5.2 to about 6.4. In an embodiment, the protein content of the composition or formulation comprises an isoelectric point (pI) of from about 5.4 to about 6.3. In an embodiment, the protein content of the composition or formulation comprises an isoelectric point (pI) of from about 5.5 to about 6.2. In an embodiment, the protein content of the composition or formulation comprises an isoelectric point (pI) of from about 5.6 to about 6.1. In an embodiment, the protein content of the composition or formulation comprises an isoelectric point (pI) of from about 5.7 to about 6.0. In an embodiment, the protein content of the composition or formulation comprises a minimum isoelectric point (pI) of from about 5.4 to about 5.6. In an embodiment, the protein content of the composition or formulation comprises a minimum isoelectric point (pI) of about 5.5. In an embodiment, the protein content of the composition or formulation comprises a main isoelectric point (pI) of from about 5.8 to about 5.9. In an embodiment, the protein content of the composition or formulation comprises a main isoelectric point (pI) of from about 5.8 to about 5.85. In an embodiment, the protein content of the composition or formulation comprises a maximum isoelectric point (pI) of from about 6.00 to about 6.10. In an embodiment, the protein content of the composition or formulation comprises a maximum isoelectric point (pI) of from about 6.00 to about 6.05. In an embodiment, the protein content of the composition or formulation comprises a maximum isoelectric point (pI) of about 6.03. In an embodiment, the protein content of the composition or formulation comprises a maximum isoelectric point (pI) of about 6.04.

In an embodiment, the protein content of the composition or formulation comprises an isoelectric point (pI) of from about 5.0 to about 6.5, as determined by Capillary isoelectric focusing (cIEF). In an embodiment, the protein content of the composition or formulation comprises an isoelectric point (pI) of from about 5.2 to about 6.4, as determined by cIEF. In an embodiment, the protein content of the composition or formulation comprises an isoelectric point (pI) of from about 5.4 to about 6.3, as determined by cIEF. In an embodiment, the protein content of the composition or formulation comprises an isoelectric point (pI) of from about 5.5 to about 6.2, as determined by cIEF. In an embodiment, the protein content of the composition or formulation comprises an isoelectric point (pI) of from about 5.6 to about 6.1, as determined by cIEF. In an embodiment, the protein content of the composition or formulation comprises an isoelectric point (pI) of from about 5.7 to about 6.0, as determined by cIEF. In an embodiment, the protein content of the composition or formulation comprises a minimum isoelectric point (pI) of from about 5.4 to about 5.6, as determined by cIEF. In an embodiment, the protein content of the composition or formulation comprises a minimum isoelectric point (pI) of about 5.5, as determined by cIEF. In an embodiment, the protein content of the composition or formulation comprises a main isoelectric point (pI) of from about 5.8 to about 5.9, as determined by cIBF. In an embodiment, the protein content of the composition or formulation comprises a main isoelectric point (pI) of from about 5.8 to about 5.85, as determined by cIEF. In an embodiment, the protein content of the composition or formulation comprises a maximum isoelectric point (pI) of from about 6.00 to about 6.10, as determined by cIEF. In an embodiment, the protein content of the composition or formulation comprises a maximum isoelectric point (pI) of from about 6.00 to about 6.05, as determined by cIEF. In an embodiment, the protein content of the composition or formulation comprises a maximum isoelectric point (pI) of about 6.03, as determined by cIEF. In an embodiment, the protein content of the composition or formulation comprises a maximum isoelectric point (pI) of about 6.04, as determined by cIEF.

In an embodiment, the composition or formulation comprises a level of protease activity of less than about 5 nKat/L. In an embodiment, the composition or formulation comprises a level of protease activity of less than about 4 nKat/L. In an embodiment, the composition or formulation comprises a level of protease activity of less than about 3 nKat/L.

In an embodiment, the composition or formulation comprises a level of prekallikrein activity of less than about 30 IU/mL. In an embodiment, the composition or formulation comprises a level of prekallikrein activity of less than about 25 IU/mL. In an embodiment, the composition or formulation comprises a level of prekallikrein activity of less than about 20 IU/mL.

In an embodiment, the composition or formulation comprises a tri (n-butyl) phosphate (TnBP) content of less than about 10 μg/mL. In an embodiment, the composition or formulation comprises a TnBP content of less than about 8 μg/mL. In an embodiment, the composition or formulation comprises a TnBP content of less than about 7 μg/mL. In an embodiment, the composition or formulation comprises a TnBP content of less than about 6 μg/mL. In an embodiment, the composition or formulation comprises a TnBP content of less than about 5 μg/mL. In an embodiment, the composition or formulation comprises a TnBP content of less than about 4 μg/mL. In an embodiment, the composition or formulation comprises a TnBP content of less than about 3 μg/mL. In an embodiment, the composition or formulation comprises a TnBP content of less than about 2 μg/mL.

In an embodiment, the composition or formulation comprises a PS80 content of less than about 20 mg/mL. In an embodiment, the composition or formulation comprises a PS80 content of less than about 18 mg/mL. In an embodiment, the composition or formulation comprises a PS80 content of less than about 16 mg/mL. In an embodiment, the composition or formulation comprises a PS80 content of less than about 14 mg/mL. In an embodiment, the composition or formulation comprises a PS80 content of less than about 12 mg/mL. In an embodiment, the composition or formulation comprises a PS80 content of less than about 10 mg/mL. In an embodiment, the composition or formulation comprises a PS80 content of less than about 8 mg/mL.

Also contemplated herein are compositions or formulations comprising a combination of any two or more of the characteristics described herein. By way of non-limiting examples, the composition or formulation described herein can comprise a combination of any two or more of the following characteristics:
(a) a hemopexin content of from about 95 mg/mL to about 110 mg/mL;
(b) heme binding activity of from about 1000 μM to about 2000 μM;
(c) heme specific binding activity of at least about 80% of total protein
(d) a CD91 dissociation constant (KD) of from about 0.50 μM to about 2.0 μM;
(e) a transferrin content of less than about 0.50 mg/mL;
(f) an albumin content of less than about 0.05 mg/mL;
(g) a haptoglobin content of less than about 0.05 mg/ml;
(h) an Apo-A1 content of less than about 0.10 mg/mL;
(i) a high molecular weight (HMW) hemopexin aggregate content of less than about 1.0% of total protein, as determined by size exclusion-high-performance liquid chromatography;
(j) a hemopexin monomer content of at least about 90% of total protein, as determined by size exclusion-high-performance liquid chromatography;
(k) a low molecular weight (LMW) impurity content of less than about 1.0% of total protein, as determined by size exclusion-high-performance liquid chromatography;
(l) a hemopexin purity content of at least about 80% of total protein, as determined by reduced SDS-PAGE or as determined by non-reduced SDS-PAGE;
(m) an isoelectric point (pI) of from about 5.0 to about 6.5, as determined by Capillary isoelectric focusing (cIEF);
(n) a level of protease activity of less than about 5 nKat/L;
(o) a level of prekallikrein activity of less than about 30 IU/mL;
(p) a tri (n-butyl) phosphate (TnBP) content of less than about 10 μg/mL; and
(q) a PS80 content of less than about 20 mg/mL.

In an embodiment, the composition or formulation described herein comprises a combination of any two or more of the following characteristics:
(a) a hemopexin content of from about 95 mg/mL to about 110 mg/mL;
(b) heme binding activity of from about 1600 μM to about 1800 μM;
(c) heme specific binding activity of at least about 97% of total protein;
(d) a CD91 dissociation constant (KD) of from about 1.10 μM to about 1.20 μM;
(e) a transferrin content of less than about 0.25 mg/ml;
(f) an albumin content of less than about 0.009 mg/mL;
(g) a haptoglobin content of less than about 0.03 mg/mL;
(h) an Apo-A1 content of less than about 0.06 mg/mL;
(i) a high molecular weight (HMW) hemopexin aggregate content of less than about 0.6% of total protein;
(j) a hemopexin monomer content of at least about 99% of total protein;
(k) a low molecular weight (LMW) impurity content of less than about 0.4% of total protein;
(l) a hemopexin purity content of at least about 88% of total protein;
(m) an isoelectric point (pI) of from about 5.4 to about 6.3;
(n) a level of protease activity of less than about 3 nKat/L;
(o) a level of prekallikrein activity of less than about 20 IU/mL;
(p) a tri (n-butyl) phosphate (TnBP) content of less than about 5 μg/mL; and
(q) a PS80 content of less than about 18 mg/mL.

In an embodiment, the composition or formulation described herein comprises (i) a hemopexin content of from about 95 mg/mL to about 110 mg/mL, (ii) a hemopexin monomer content of at least about 99% of total protein, and (ii) a heme specific binding activity of at least about 97% of total protein. In another embodiment, the composition or formulation further comprises (i) a transferrin content of less than about 0.25 mg/mL and (ii) a haptoglobin content of less than about 0.03 g/L haptoglobin.

In an embodiment, the composition or formulation described herein further comprises no detectable apolipoprtein A1 and/or albumin content. In an embodiment, the composition or formulation described herein further comprises (i) an albumin content of less than about 0.009 mg/mL, and (ii) an Apo-A1 content of less than about 0.06 mg/mL.

In an embodiment, the composition or formulation described herein further comprises no detectable protease activity. Suitable methods of determining protease activity will be familiar to persons skilled in the art, illustrative examples of which are described, for example, in Zhang et al (editors. Assay Guidance Manual [Internet]. Bethesda (MD): Eli Lilly & Company and the National Center for Advancing Translational Sciences; 2004), the entire contents of which is incorporated herein by reference).

In an embodiment, the composition or formulation described herein is suitable for pharmaceutical administration after storage for 12 months at 2° C. to 8° C. and/or at ambient (e.g. room) temperature.

### Methods of Treatment

In another aspect of the present invention, there is provided a method of treating a condition associated with haemolysis, the method comprising administering to a subject in need thereof the composition or the formulation of the present invention, as disclosed herein.

The term “subject”, as used herein, refers to an animal which includes a primate (a lower or higher primate). A higher primate includes human. Whilst the present invention has particular application to targeting conditions in humans, it would be understood by those skilled in the art that non-human animals may also benefit from the compositions and methods disclosed herein. Thus, it will be appreciated by the skilled addressee that the present invention has both human and veterinary applications. For convenience, an “animal” includes livestock and companion animals such as cattle, horses, sheep, pigs, camelids, goats, donkeys, dogs and cats. With respect to horses, these include horses used in the racing industry as well as those used recreationally or in the livestock industry.

The compositions or formulations comprising hemopexin, as described herein, may be administered to the subject by any suitable route. Illustrative examples of suitable routes of administration include intravenous, subcutaneous, intra-arterial or by infusion. In an embodiment, the compositions or formulations described herein are administered intravenously.

Where necessary, the methods of treatment described herein may further comprise administering a second therapeutic agent. The second therapeutic compound may be co-administered to the subject sequentially (before or after administration of the compositions or formulations disclosed herein) or concurrently. In an embodiment, the second therapeutic agent is an iron chelating agent (e.g., deferrioxamine or deferiprone).

In another aspect disclosed herein, there is provided use of the compositions or formulations comprising hemopexin, as described herein, in the manufacture of a medicament for treating a condition associated with haemolysis. In an embodiment, the compositions or formulations described herein are formulated for use in humans.

The compositions and formulations described herein are particularly suitable for treating a subject with a condition associated with haemolysis, including those associated with a risk of haemoglobin/heme-mediated toxicity. Conditions associated with haemolysis, including those associated with a risk of haemoglobin/heme-mediated toxicity, are known in the art. In an embodiment, the condition is selected from an acute haemolytic condition and/or a chronic haemolytic condition. In an embodiment, the condition is selected from the group consisting of haemolytic anaemia, transfusion-induced haemolysis, haemolytic uraemic syndrome, an autoimmune disease, malaria infection, trauma, blood transfusion, open heart surgery using cardiopulmonary bypass and burns, including in the treatment of hemoglobinemia or hemoglobinuria accompanied with hemolysis after burn. In an embodiment, the condition is selected from the group consisting of sickle cell anaemia, hereditary spherocytosis, hereditary elliptocytosis, thalassemia, congenital dyserythropoietic anemia and Paroxysmal nocturnal hemoglobinuria, systemic lupus erythematosus and chronic lymphocytic leukemia.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1—Screening of Mixed-Mode Chromatography Resins for Hemopexin Purification

Initial investigations found that the isoelectric points of hemopexin, and the major contaminating proteins in FIV-4 were all quite similar, ranging from around pI 4.8 to 6.3. This indicated that ion-exchange chromatography was unlikely to provide a powerful purification step for hemopexin. As an alternative, mixed mode chromatography resins were investigated leveraging their ability to allow separation by hydrophobic interaction as well as by charge. In an initial resin screening study, several mixed-mode cation and anion exchange resins were screened in a high throughput format, and their utility for hemopexin purification was assessed.

### Mixed-Mode Cation Exchange

The mixed mode cation exchange resins Capto MMC (Cytiva), Eshmuno HCX (Merck), Nuvia cPrime (Bio-Rad) and Toyopearl MX-TRP 650M (Tosoh) were screened in this study.

FIV-4 paste was resuspended in acetate buffer at pH 4.5 and loaded onto mixed mode cation exchange columns equilibrated in the same buffer. Unbound material was washed through with the same buffer and bound proteins eluted with a stepwise NaCl gradient.

No reasonable selectivity towards hemopexin was observed with Eshmuno HCX or Toyopearl MX-TRP 650M resins, with the majority of all proteins eluting in unbound fractions. Conversely, no proteins were eluted from Capto MMC or Nuvia cPrime, indicating strong retention of all proteins. Given the expectation (based on pI) that hemopexin should be one of the more strongly retained proteins, these resins were identified as having potential for a suitable hemopexin capture step. Further development of these resins was performed to establish whether sufficient selectivity could be achieved for effective purification (Example 4).

### Mixed-Mode Anion Exchange

The mixed mode anion exchange resins; Capto Adhere (Cytiva), MEP Hypercel (Sartorius), HEA Hypercel (Sartorius) and PPA Hypercel (Sartorius) were screened in this study.

FIV-4 paste was resuspended in phosphate buffer at pH 7.5 and loaded onto mixed mode anion exchange columns equilibrated in the same buffer. Unbound material was washed through with the same buffer and bound proteins eluted with a stepwise NaCl gradient. In a second experiment, paste was resuspended at pH 7.5 in 140 mM NaCl/30 mM KCl, applied to anion exchange columns, and eluted with a stepwise decreasing pH gradient.

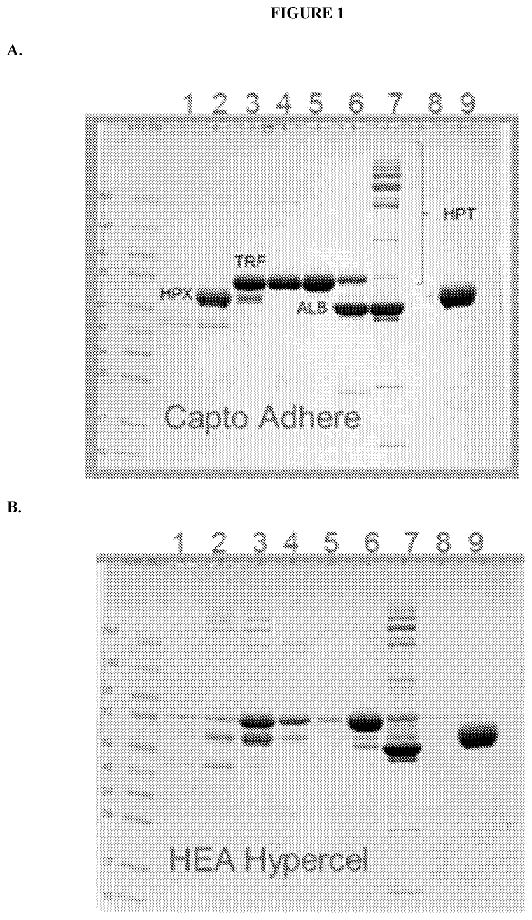

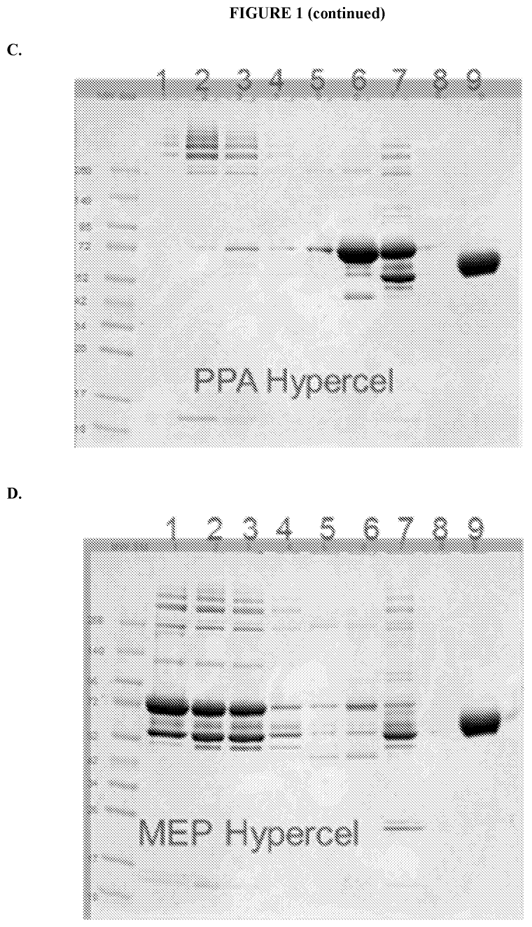

The mixed mode resins MEP Hypercel, HEA Hypercel and PPA Hypercel showed poor selectivity, with hemopexin eluting along with contaminant proteins in several fractions (FIG. 1).

When loaded onto Capto Adhere resin in the presence of salt, hemopexin was found at reasonably high purity in the unbound fraction. Whilst this clearly indicates a useful purification step, when loaded with paste extract the binding of all other proteins would lead to low hemopexin capacity, with the column rapidly saturating with contaminants. This suggests that Capto Adhere chromatography may be best suited for use as a second purification step, once most contaminant proteins have been removed.

Further studies performed to optimise conditions for downstream purification with Capto Adhere are discussed in Example 5.

### Additional Cation/Anion Exchange Resins

Initial studies centred on purification of hemopexin from extracted FIV-4 paste, and involved screening of the cation exchange resins Eshmuno CPX, Eshmuno S, Eshmuno COO and Fractogel EMD COO⁻ (M). The Eshmuno CPX and Eshmuno COO resins both demonstrated binding selectivity for hemopexin, but recovery was poor, with 40% and 20% hemopexin present in the flow through fraction respectively, and low binding capacity observed for both resins. As such, these cation exchange resins were not considered to be suitable as a first purification step.

The feasibility of anion exchange chromatography resins, Eshmuno Q, Fractogel TMAE Hicap (M) and Fractogel DMAE (M), was investigated for their potential use as a polishing step. This study was performed using hemopexin containing only minor amounts of contaminating protein, as expected following partial purification. Of these, Eshmuno Q produced favourable results, but further development of this resin was discontinued in favour of cation exchange resins.

When the cation exchange chromatography resins, Eshmuno COO; Eshmuno CPS, Eshmuno CPX and Fractogel SE HC (M) were screened, Eshmuno CPS was identified as a viable option for a polishing step. It was identified that in the final stages of the hemopexin purification process, hemopexin would likely be substantially pure, with some contaminating transferrin remaining. Further development work therefore used a surrogate feed mixture of 5 mg/mL hemopexin and 0.5 mg/mL transferrin. These further studies demonstrated that at pH 6.0, and 10 mS/cm conductivity, Eshmuno CPS resin had high binding capacity for hemopexin, (around 40 mg/mL resin), and was able to remove around 80% of the contaminating transferrin.

It was anticipated that this chromatography step would be well placed as a final polishing step in the hemopexin purification process. In the overall process design, this would likely result in the feed for this column containing 1% polysorbate 80 (PS80) and 0.3% tri (n-butyl) phosphate (TnBP) following viral inactivation. Using the conditions described above and partially purified intermediate as the load material, the solvent/detergent reagents were shown to have no effect on the separation characteristics of Eshmuno CPS. The solvent/detergent reagents eluted in the flow through during the loading and wash steps, resulting in low concentrations of PS80 and TnBP in the hemopexin product. This indicated that Eshmuno CPS resin was a viable option for both purity polishing and removal of solvent/detergent from the product.

As Eshmuno CPS chromatography resin was not tested during the initial screening of cation exchange resins using paste extract, and as it demonstrated high binding selectivity for hemopexin, it was re-assessed for potential use as an initial purification step. When extracted paste was used as the load material, constant breakthrough of hemopexin was observed during loading, with approximately 40% of hemopexin lost regardless of load conductivity (5 and 10 mS/cm). This may be indicative of competition for binding sites with another component in crude extract and/or the presence of different forms of hemopexin, such as heme complex. Regardless, further development of the Eshmuno CPS resin as an initial purification step was not pursued due to low recovery.

Eshmuno CPS chromatography resin was further optimised for use as a polishing step following viral inactivation. These studies are described in Example 7.

### Example 2—Evaluation of Extraction Conditions for Fraction IV-4 Paste for Hemopexin Purification

The first step in the purification process of hemopexin from FIV-4 paste is a resuspension in an aqueous buffer. As several different proteins are present in the FIV-4 paste, certain extraction conditions may preferentially solubilise hemopexin, whilst leaving some or all of the contaminating proteins in the insoluble fraction. Solubilisation conditions that maximise the amount of hemopexin extracted from the FIV-4 paste, and provide the protein in a matrix compatible with clarification and further downstream purification were required. From a practical standpoint, it is preferable that solubilisation be performed in relatively small volumes to facilitate large-scale manufacture. With these goals in mind, a number of extraction buffers and conditions were investigated to optimise the extraction process step. These studies are described briefly below.

### Initial FIV-4 Paste Extraction Development for Laboratory Scale Batches

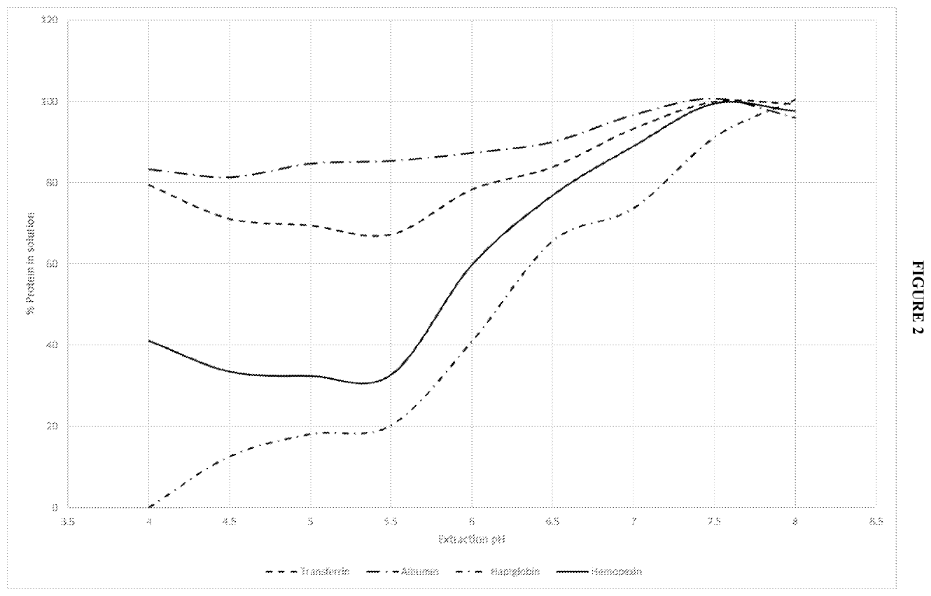

Initial extraction studies, performed over the pH range pH 4 to pH 8 demonstrated that only small amounts of hemopexin and haptoglobin were solubilised at pH <5.5, with a large amount of albumin and transferrin present in the extraction solution across all pH levels (FIG. 2). Under the pH range investigated, no conditions were identified that facilitated the preferential extraction of hemopexin.

Extraction of hemopexin was demonstrated to be most effective and high yielding at neutral, or near neutral pH. Hemopexin was not preferentially extracted at this condition, with most contaminating protein also extracted under these conditions. Efficient resuspension of FIV-4 paste was demonstrated at paste: buffer ratios from 1:5 up to 1:20 and mixing times from one to four hours, with comparable hemopexin intermediate produced over the combination of these conditions.

Following the initial extraction process development studies, laboratory scale batch manufacture using the second generation hemopexin process (Example 13) was performed utilising extraction conditions at pH 6.5 with 250 mM NaCl, paste: buffer ratio of 1:10 and a two hour mixing time.

### FIV-4 Paste Batch Consistency

During early experiments, the hemopexin concentration of the extracted FIV-4 paste solution was observed to vary by more than 50%. It was hypothesised that this variation was due to batch variation of the FIV-4 paste.

To examine this, the consistency of FIV-4 paste extraction and depth filtration was explored over 10 batches of FIV-4 paste. The experiment was performed with 0.2 kg paste from each batch, solubilised at a 1:10 paste: buffer ratio in 40 mM phosphate, 180 mM NaCl, pH 6.2 for 2 hours at room temperature. The extract was clarified by filtration with 3M 90LP depth filters with a filter area of 0.04 m², pre-coated with 625 g/m² filter area Celpure C1000 and one filter volume post wash (Example 3). The hemopexin concentration of the extract was measured by reversed-phase HPLC before and after depth filtration.

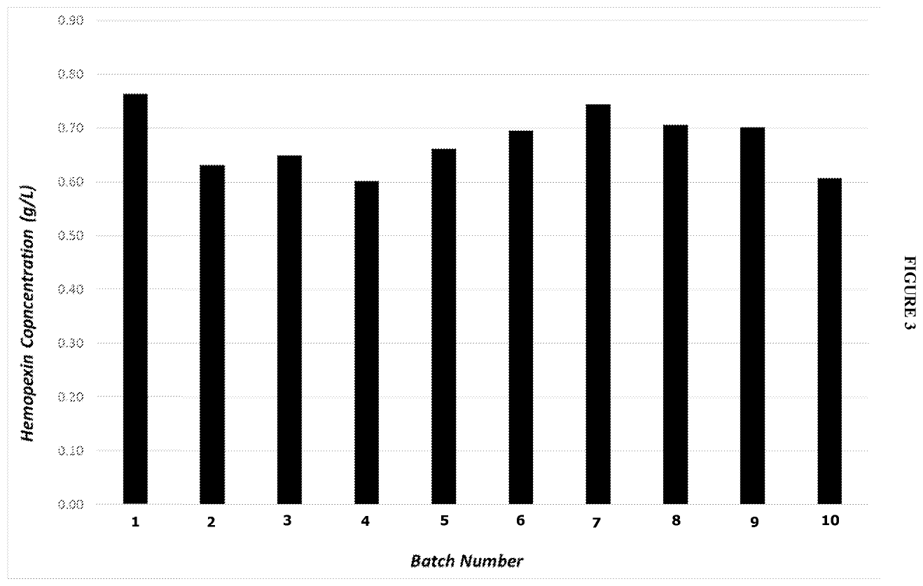

When analysed by reversed-phase HPLC, the hemopexin concentration of extracted paste varied from 0.6 to 0.76 g/L (FIG. 3). Performance over the depth filtration process step was consistent across the batches tested with hemopexin recovery of approximately 80 to 95% regardless of the method of quantitation.

### Optimisation of FIV-4 Paste Extraction for Pilot Scale Manufacture

Despite early studies showing maximal extraction of hemopexin at pH 7.5, extraction for laboratory studies was performed at pH 6.2 to closely match the equilibration conditions for the first chromatography step. To improve yields at pilot scale, a study was performed to investigate whether extraction at pH 7.5 extracted sufficiently more hemopexin to justify the extra manipulation involved in pH adjustment post-clarification.

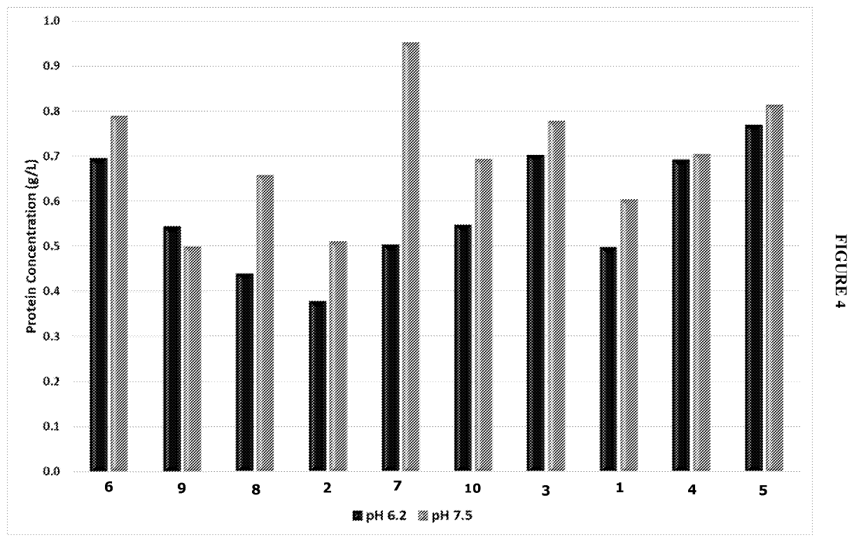

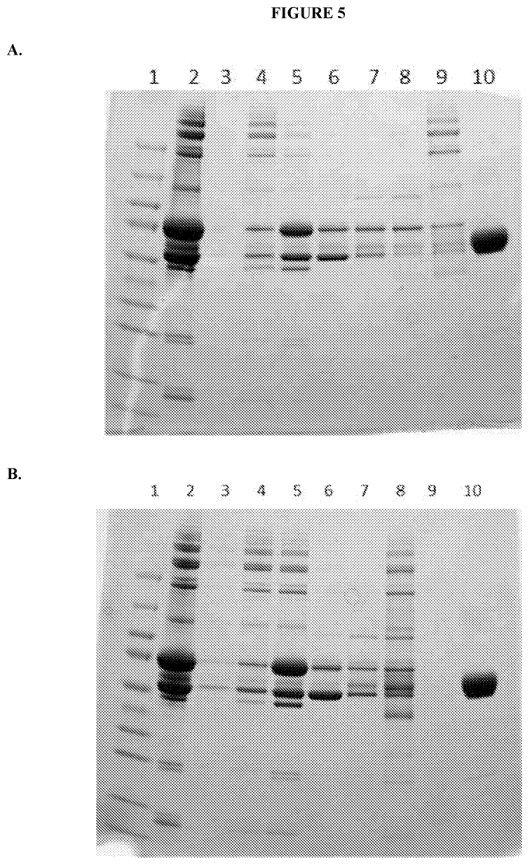

Hemopexin extractions were performed with 10 batches of FIV-4 paste at pH 6.2 and 7.5. Extractions were performed at a 1:10 paste: buffer ratio in 40 mM phosphate, 180 mM NaCl at the given pH for 1 hour at room temperature. It was demonstrated that by increasing the extraction pH to 7.5, around 20% more hemopexin could be solubilised, regardless of the paste batch (FIG. 4). This was considered a significant increase in yield.

As part of the Capto MMC chromatography process development, robustness studies around loading conditions were performed and a NaCl concentration of 225 mM was identified as the target loading condition (Example 4).

Following these studies, extraction buffer conditions were changed to 225 mM NaCl to match conductivity with Capto MMC equilibration, and pH 7.5 to maximize yield. These conditions were used for the remaining laboratory scale and pilot scale batches (Examples 13 and 14).

Prior to pilot scale batch manufacture, solubilisation of FIV-4 paste in smaller amounts of extraction buffer was investigated in order to minimise tank sizes in a manufacturing setting. Hemopexin extraction performed at paste: buffer ratios of 1:2.5 and 1:5 demonstrated that comparable amounts of hemopexin were extracted, ranging from 5.5-6.6 g per kg paste. Concerns were initially raised that residual ethanol in paste may lead to low recoveries from the first chromatographic purification step, Capto MMC resin, when solubilisation ratios are low. Clarified extracted paste from the 1:2.5 1:5 and 1:10 extractions was subjected to Capto MMC chromatography and demonstrated that lower ratios had no impact on purity and recovery, indicating no notable effect of residual ethanol, and suitability of extracts at ratios as low as 1:2.5. Studies performed with an extraction paste: buffer ratio of 1:1, showed lower recovery of hemopexin and protein over the Capto MMC chromatography step.

At lower extraction ratios, a larger filter press post-wash volume was required to maximise protein recovery, up to 3× press volumes required at a 1:2.5 extraction ratio.

### Optimisation for FIV-4 Paste Extraction for Manufacturing Scale

At manufacturing scale, it is likely that paste will be frozen before use and in this case, solubilisation directly from frozen paste would provide simpler paste handling. To assess this, the solubilisation efficiency of hemopexin was assessed after extraction of frozen paste and paste thawed at 2-8° C., resuspended in buffer at room temperature (approximately 22° C.) and 4° C. The results demonstrated that extraction efficiency with frozen paste was slightly higher than that obtained with thawed paste, regardless of the extraction buffer temperature. As paste was stored for several days at 4° C. before extraction, this suggests that there may be some degradation of hemopexin in FIV-4 paste under this storage condition. Recovery over depth filtration was demonstrated to be consistent regardless of the paste and extraction buffer temperature.

As part of further optimisation of the process for manufacturing scale, the NaCl concentration of the extraction buffer was modified in order to achieve a conductivity for the clarified extract paste equivalent to the equilibration buffer for the Capto MMC column. Performing FIV-4 paste extraction in a buffer containing 400 mM NaCl produced a clarified extracted paste process intermediate with a conductivity of 27 mS/cm, comparable to the Capto MMC equilibration buffer. This removes the requirement to perform a conductivity adjustment after depth filtration and clarification.

### Final Process Extraction Method

The hemopexin containing FIV-4 paste is resuspended in a 40 mM sodium phosphate, 400 mM sodium chloride pH 7.5±0.1 extraction buffer at a 1:2.5 w/w ratio of paste to buffer. Paste is broken into small clumps and added slowly to the extraction buffer. The extract is stirred for at least 120 minutes at room temperature before clarification, with at least 60 minutes of stirring after all visible clumps have dissociated.

### Example 3—Evaluation of Fraction IV-4 Paste Clarification Conditions for Hemopexin Purification

Development of an efficient filtration step was necessary prior to the downstream chromatographic purification process steps to remove suspended particulate matter and prevent column fouling. Following development of initial conditions for solubilisation of hemopexin from FIV-4 paste, clarification process development was performed in conjunction with further extraction studies, to ensure compatibility of extraction and clarification conditions. Depth filtration and clarification studies were performed with the understanding that a filter press will be utilised for depth filtration at manufacturing scale. These studies are described briefly below.

### Initial Development of Depth Filtration and Clarification

Several different depth filter media from manufacturers 3M and Pall were screened for optimal throughput and clarification of extracted FIV-4 paste. Fraction IV-4 paste was resuspended in phosphate buffer, 250 mM NaCl, at pH 6.5 at a 1:10 paste: buffer ratio. Extracts were clarified using 47 mm disc filters, 60 mM disc filters, or small cartridges under a constant pressure of 2 bar.

Acceptance criteria for filter performance were initially set with a throughput of at least 200 L/m² filter area, and turbidity of less than 80 NTU. The NTU specification was based upon initial studies that found this level of turbidity after 0.22 μm filtration of extracts, and no apparent column fouling with this material.

Dual layer cartridge filters and sandwich filters provided some of the highest levels of clarification, but had very low throughput characteristics due to clogging of the cartridges with residual filter aid from the original FIV-4 paste. A two filtration step process, which included a first coarse filter such as 3M 30SP filter sheet followed by a second depth filter sheet of finer grade, was shown to produce a high clarity product intermediate with high throughput. However, in a manufacturing setting, a two-step or sandwich filter filtration process is undesirable.

When used as a single layer filter, the 3M zeta plus 90SP filter media was selected to produce a product intermediate with reasonable clarity and throughput.

The best throughput and clarification was achieved when the depth filter was pre-coated with filter aid. Addition of filter aid into the extracted paste feed was less efficient, with reduced clarification and lower throughput observed compared to pre-coating the filter with filter aid (premixed aid). There was little difference in depth filtration performance using two different filter aids, Celpure C1000 and Celpure C300.

Filter press capacity studies identified a filter area of 0.11 m²/kg paste and 4 cm frame depths provided adequate capacity to perform depth filtration of the extracted paste solution.

A relatively high flow rate was required to produce an even coat of filter aid and filter cake in the filter press. At low flow rates, filter aid was observed to pool in the bottom of the filter frame, reducing filter efficiency and contributing to fouling of inlet lines. Studies performed identified that a flow rate of 6.25 L/m² filter area/min produced an even coat of filter aid on the filter sheet.

Following initial development of the depth filtration step, further studies were performed with 1:10 extraction ratios, using 3M 90LP depth filters with a filter area of 0.11 m²/kg paste, pre-coated with 625 g/m² filter area Celpure C1000 and one filter volume post wash.

### Optimisation of Depth Filtration and Clarification

Variation in recovery over the depth filtration step was observed in laboratory scale batches, with one particular batch recovering only 50 to 60% hemopexin over this step (Example 13). Investigation of the cause showed that recovery was significantly worse when filtration was performed without a post-wash step. Lower extract protein concentration, alternative extraction buffers, or post-washing with high salt had little or no impact on product recovery. Taken together, this indicates that losses over the depth filtration step were unlikely to be due to protein binding to the filter, and are most likely due to inadequate post-wash volume.

The optimal volume of depth filter post-wash for a 1:10 paste: buffer extraction was determined using a 20×20 cm filter press with a filter area of 0.012 m²/L extract and the results indicated that almost two press-volumes were required to recover all of the protein.

To be better suited to large scale manufacture, the FIV-4 paste extraction conditions were optimised to reduce the volume, with extraction performed at a paste: buffer ratio of 1:2.5. When extraction was performed at the 1:2.5 ratio, the filter area used was scaled according to the amount of paste used, such that the solid load per unit area was the same as at the 1:10 extraction. This equated to 9 kg paste/m² filter area or 0.031 m²/L extract. Filtration efficiency for the 1:2.5 extraction ratio was not substantially different from a 1:10 extraction, however, a larger post-wash volume of 3 press volumes was required to recover most of the protein.

### Estimation of Hemopexin Concentration in Clarified Extract

To calculate the appropriate load onto the Capto MMC chromatography resin, measurement of the hemopexin concentration of the clarified extracted paste solution is required. At the extracted paste solution stage, hemopexin makes up a low proportion of the total protein content. Immunonephelometry or reversed-phase HPLC were therefore used during early development to provide the hemopexin concentration. During manufacture, it is not feasible to perform a complicated or time consuming method to provide a concentration for loading onto the Capto MMC resin and, hence, a simple in-process test method was developed using the OD₂₈₀ₙₘ of the extract solution.

The OD₂₈₀ₙₘ data from the FIV-4 paste batch consistency study (Example 4) was compared with the measured hemopexin concentration of each batch by reversed-phase HPLC. It was found that, on average, the hemopexin concentration could be estimated by dividing the OD₂₈₀ₙₘ by 13 (Table 1Error! Reference source not found.).

### Final Clarification Method

The FIV-4 extracted paste solution is clarified by 3M 90SP zeta plus depth filter media in a filter press with filter area of 1 m²/9 kg paste, using a 4 cm frame depth. The filter press is pre-washed with one press volume of extraction buffer containing 625 g of Celpure 1000 filter aid/m² of filter area, at a flow rate of 6.25 L/m²/min. Filtration of the FIV-4 extracted paste solution is then performed at a flow rate of 6.25 L/m²/min and this typically results in a pressure of <1.5 bar (max 2 bar) during filtration. The filter press is post-washed with 3 press volumes of Capto MMC equilibration buffer. The resulting depth filtered extract is pH adjusted to pH 6.4±0.1 with 0.5 M HCl and conductivity confirmed to be 26 to 28 mS/cm. The depth filtered extract is then further clarified using a Millipak 200 0.2 μm cartridge or similar, with an area of 33 cm²/L to produced clarified extracted paste.

### Example 4—Mixed-Mode Chromatography with Capto MMC Resin

Two mixed-mode resins, Capto MMC and Nuvia cPrime, were optimised and compared for use as the first purification step in hemopexin manufacturing process described herein. Initial studies demonstrated that there was little difference in the yield and purity of hemopexin purified by Capto MMC and Nuvia cPrime resins. However, after several studies to optimise performance of these two resins, Capto MMC resin demonstrated marginally better purity than the Nuvia cPrime resin. Studies performed in the development and optimisation of the Capto MMC resin are summarised below.

### Development and Optimisation of Capto MMC Chromatography

Early studies showed that Capto MMC resin binds all proteins very strongly at low pH (Example 1). It was hypothesised that if hemopexin was among the more strongly bound proteins, conditions could be established whereby Capto MMC could constitute an effective capture step.

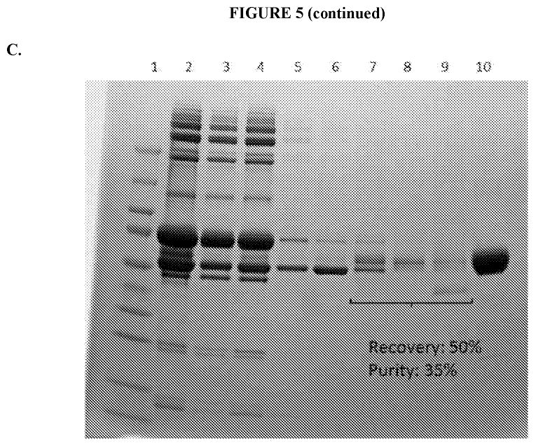

An initial assessment of the binding conditions, was made over the pH range pH 5.0 to 7.0, with the goal of maximizing hemopexin binding whilst minimizing contaminant binding. At each pH, elution conditions were assessed at a range of NaCl concentrations (50 mM, 150 mM, 300 mM, 500 mM and 1 M). The highest contaminant levels were observed in the unbound fraction for loading at pH 7.0. Whilst some hemopexin did not bind to the Capto MMC resin and was observed in the unbound fraction, around 50% of the loaded hemopexin was eluted in the combination of higher NaCl concentrations, at a purity level of ˜30% (FIG. 5).

Based on the results of the initial study, it was determined that optimal binding conditions required further manipulation of either pH, NaCl concentration, or both. A study was therefore performed investigating binding conditions between pH 6.0 and 7.5 at a range of NaCl concentrations, with elution conditions between pH 7.0 and pH 7.5.

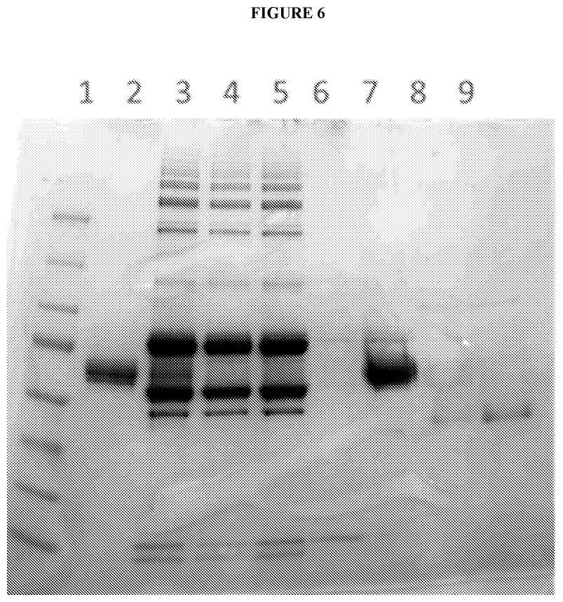

Loading the hemopexin starting material in 200 mM NaCl at either pH 6.0 or pH 6.5, and elution at pH 7.5 resulted in high recovery of hemopexin, at ≥90%. However, higher purity (˜90%) was observed when loading at pH 6.5, compared to 58% purity at pH 6.0. Under the pH 6.5, 200 mM NaCl loading conditions, hemopexin appeared to be almost the only protein bound, with most other proteins eluting in the unbound fraction (FIG. 6). Clearly, these loading and elution conditions are highly suited to the purification of hemopexin using Capto MMC resin.

With the binding and elution conditions for hemopexin purification established, the hemopexin binding capacity of Capto MMC resin, and appropriate load amounts were determined. Clarified extracted paste was loaded onto a 5 mL Capto MMC column, to a total hemopexin load of 250 mg per mL resin with a contact time of 7.5 minutes. Fractions were collected during the loading step, and these were analysed for hemopexin concentration by immunonephelometry.

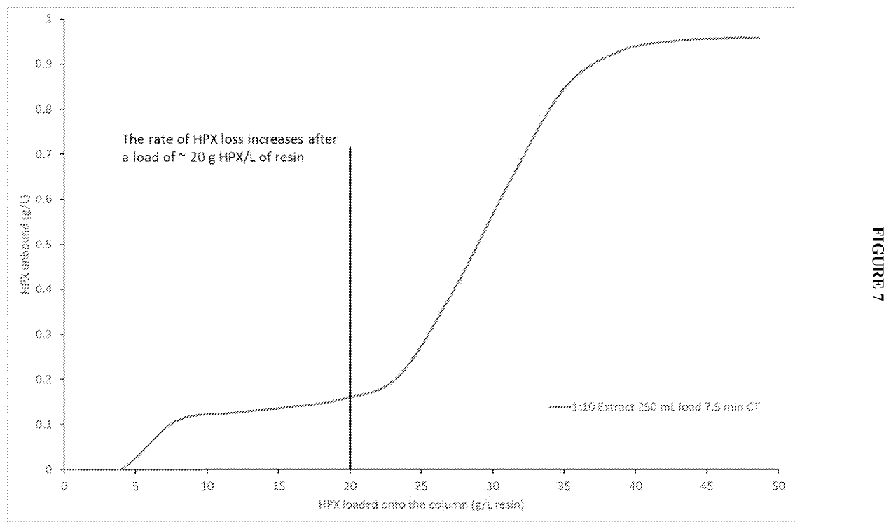

After loading around 4 mg hemopexin per mL resin, a small amount of hemopexin was observed in the unbound fraction. This amount remained fairly constant until around 20 mg of hemopexin had been loaded per mL of resin, after which, the amount of hemopexin breakthrough increased substantially (FIG. 7). This indicated the presence of a proportion of hemopexin that does not bind to Capto MMC under the current conditions, with the remaining hemopexin able to be captured to a capacity of around 20 mg per mL resin, with some modest losses.

This is consistent with the presence of two populations of hemopexin with different physicochemical characteristics, and the presence of heme-hemopexin complex was suspected (Example 10).

The optimal hemopexin load amount for the Capto MMC chromatography process step was further investigated by loading different amounts of clarified extracted paste, and quantifying the eluted hemopexin. The eluted amounts generally corresponded with the reverse of that seen in the unbound fraction. Recoveries were reasonably consistent at 70-75% until the hemopexin load amount exceeded about 15 mg per mL resin, falling to around a 65% hemopexin recovery at a 20 mg per mL resin load. Based on this, the optimal load amount was determined to be 14 mg per mL resin, with 20 mg per mL resin considered the maximum acceptable load.

Again, a constant recovery of 70-75% initially, points to the presence of a second population of hemopexin with weaker binding characteristics (Example 10).

In other brief studies, the effect on column capacity was assessed with different load contact times. These studies showed that a contact time of 3 minutes produced very similar results to those outlined above with 7.5 minutes contact time. Whilst this may allow a more rapid loading of feed, it was decided that a contact time of 7.5 minutes with a bed height of 15 cm would allow the entire method to be run at a linear velocity of 120 cm/hr, which is comfortably achievable with most instrumentation and avoids potential overpressure issues with higher flow rates. A bed height of 15 cm also falls within a range recommended for ease of column packing at commercial scale.

### Loading Conditions

A robustness study of Capto MMC loading conditions was performed to more specifically define the optimal loading conditions, and to determine an acceptable operating range for these conditions.

Capto MMC loading conditions were investigated within the pH range of 6.0 to 6.6 and NaCl concentration of 160 to 250 mM, with the FIV-4 extraction performed in the Capto MMC equilibration buffer.

Over the Capto MMC chromatography step, purity and recovery of hemopexin were unaffected over a pH range of 6.2 to 6.6 with NaCl concentrations between 160 and 25 OmM. Material processed at pH 6.0 however, had poorer purity and recovery characteristics, with 160 mM NaCl poorer than 200 mM NaCl.

In order to determine the effect of Capto MMC loading parameters on the final product quality, material was processed using the entire putative purification process, with different Capto MMC loading conditions.

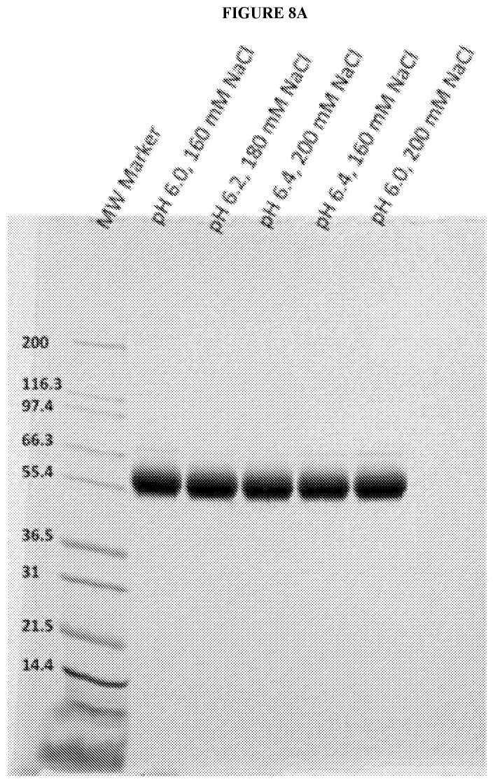

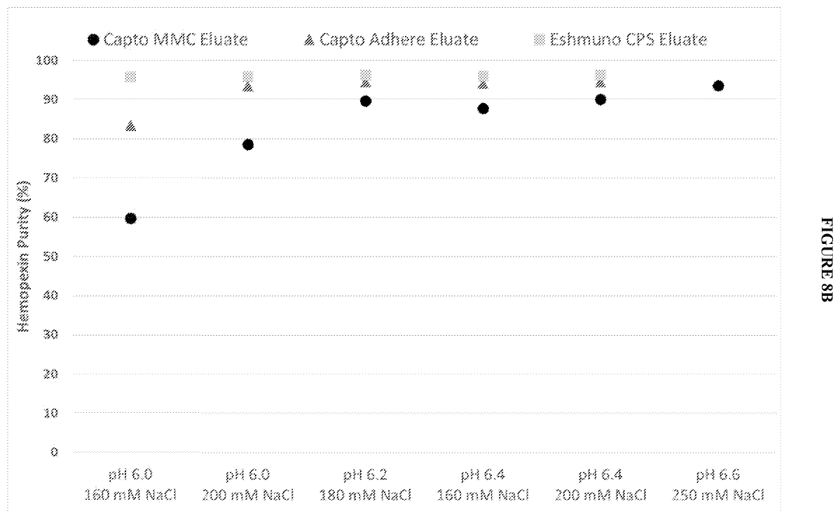

Following processing over the Capto MMC chromatography step at each loading condition (except pH 6.6, 250 mM NaCl), the hemopexin product was processed through Capto Adhere chromatography, solvent detergent treatment and Eshmuno CPS chromatography under optimal conditions for these steps (Examples 5 to 7). An increase in hemopexin purity was observed following processing over the Capto Adhere and Eshmuno CPS chromatography steps, with all conditions demonstrating equivalent final purity, at approximately 96% hemopexin by RP-HPLC (FIG. 8). All loading conditions demonstrated equivalent protein contaminant profile at the Eshmuno CPS eluate process intermediate, with only a faint transferrin band visible by non-reduced SDS-PAGE. This demonstrates that the downstream processing steps of Capto Adhere and Eshmuno CPS chromatography are robust and capable of producing an equivalent final product despite sub-optimal Capto MMC loading conditions.

From the loading condition data, the target Capto MMC chromatography loading conditions were defined as pH 6.4 and 225 mM NaCl, with a proven acceptable range of pH 6.2 to 6.6 and 200 to 250 mM NaCl.

### Stability of Capto MMC Eluate

During development, Capto MMC eluate was required as the feedstock in the assessment of other chromatography resins. This could be expedited by the purification of a large amount of material that could be stored and used in these development studies. During commercial manufacture, situations may also arise where product intermediates require storage. An understanding of the stability of stored product intermediate is therefore of considerable importance.

In order to ensure that stored material was of sufficient quality, a brief stability study was performed, and is summarised below.

Fraction 4-IV paste was extracted in 40 mM phosphate, 250 mM NaCl at pH 6.50 at a ratio of 1:10 and clarified by 0.22 μm filtration. The clarified extract was purified by Capto MMC chromatography, equilibrated in the same buffer and eluted with 40 mM phosphate, 150 mM NaCl, pH 7.5.

The partially purified hemopexin was stored at 4° C. and at room temperature for up to 7 days. Samples were taken at initiation of the study (T=0), after 2 and 7 days, and analysed for protein concentration, purity, monomer content, heme binding activity and charge heterogeneity. To ensure samples could be frozen without affecting results, a sample was subjected to a single freeze-thaw episode and analysed as above. This enabled the use of a frozen T=0 control at each time point.

The results show no notable changes to hemopexin under any of the storage conditions (see Table 2). This indicates that Capto MMC eluate can be stored for up to 7 days at room temperature or below, without affecting product quality. Alternatively, the product may be frozen at least once without detrimental effect.

### Capto MMC Chromatography Process Step

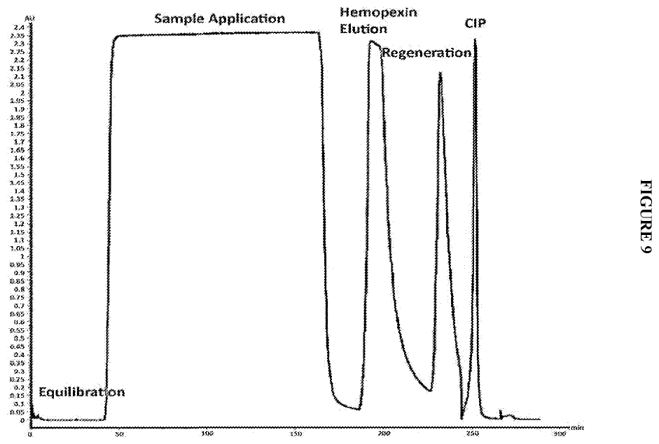

Clarified extracted paste solution (Example 3) is loaded onto a mixed mode Capto MMC chromatography column, equilibrated with 5 CV of equilibration buffer (40 mM sodium phosphate, 225 mM NaCl, pH 6.4). The product is loaded to a target load of 14 g hemopexin/L of resin. After product loading, the column is further washed with 3 CVs of equilibration buffer to remove unbound proteins. Hemopexin is eluted with 3 CVs of elution buffer (40 mM sodium phosphate, 150 M NaCl, pH 7.5). Collection of Capto MMC eluate commences 5 minutes into elution buffer application phase and continues until the UV absorbance returns to baseline. An example chromatography profile is shown in FIG. 9. The eluted hemopexin concentration is typically between 3 and 6 mg/mL and is 70 to 85% pure. Following product collection, the Capto MMC column is regenerated with 40 mM phosphate, 1M NaCl pH 7.5 buffer.

### Example 5—Development and Optimisation of Capto Adhere Chromatography

Initial chromatography screening studies identified Capto Adhere as a potential resin for hemopexin purification (Example 3). In these studies, hemopexin eluted from this resin in the unbound fraction with reasonable purity when loaded at pH 7.5 with NaCl and KCl. As hemopexin was found in the unbound fraction with most other protein binding to the column, this resin appeared to be most suited to circumstances in which the contaminant protein load is low. Further studies were therefore performed to optimise conditions for Capto Adhere as a second step, following on from partial purification by Capto MMC. Studies performed in the development and optimisation of the Capto Adhere resin are summarised below.

The initial chromatography screening studies showed that the drop through fraction of the Capto Adhere contained predominantly hemopexin with a small amount of transferrin when operated under loading conditions of 140 mM NaCl, 30 mM KCl at pH 7.5. To optimise the binding of contaminant proteins, the effect of various loading salt concentration and pH conditions was investigated.

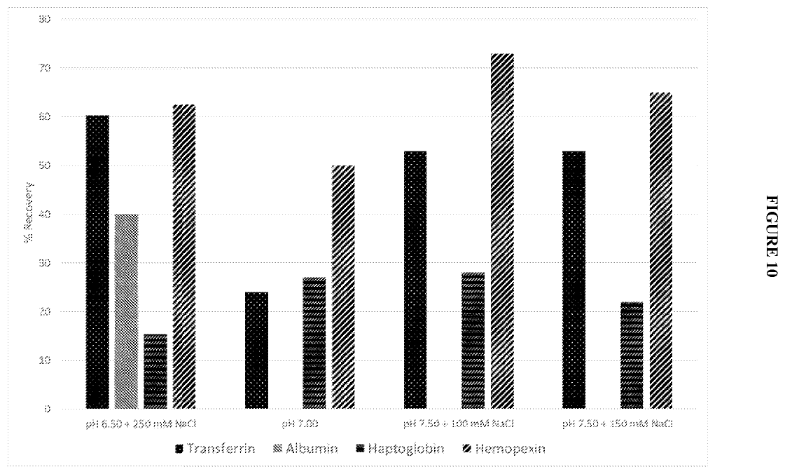

A first study was performed prior to the availability of partially purified hemopexin (i.e., Capto MMC eluate), using clarified extracted paste as the starting material. A combination of loading conditions, pH 6.5, 7.0 and 7.5 and NaCl concentrations 100, 150 and 250 mM were investigated, and the concentration of the major protein species in the unbound fraction was measured by immunonephelometry. The results show that the highest hemopexin purity and recovery was observed for the pH 7.5 and 100 mM NaCl load condition, and although most conditions were able to remove albumin quite effectively, many other species remained (FIG. 10). It is suspected that these other species remain present due to a combination of the large contaminant protein load, and potential overloading of the column. Further studies were not conducted with clarified extract as starting material.

As the Capto Adhere chromatography step appeared to be best suited as a second purification step, further development of loading conditions was performed using hemopexin after partial purification using Capto MMC resin. In this study, Capto MMC eluate was adjusted to pH 7.5 in range of NaCl concentrations and applied to a small Capto Adhere column equilibrated with the same buffer. As the feed for this experiment was partially purified material, consisting of mainly hemopexin and transferrin, only these two proteins were quantitated in the unbound fraction by immunonephelometry and SDS-PAGE was performed to assess purity. This experiment was performed as a rapid investigation, with loading contact time at 1 min for most conditions. The 150 mM NaCl condition was then repeated at pH 7.5 and 8.0 with an 8 minute contact time.

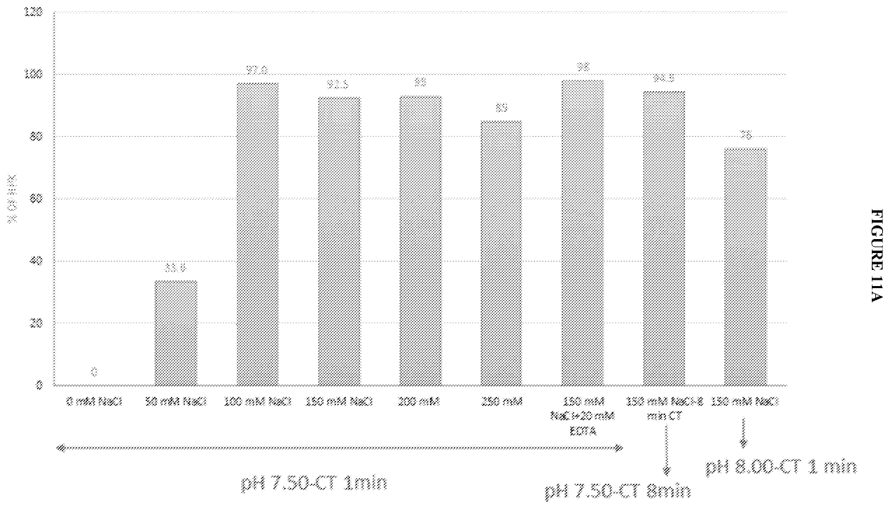

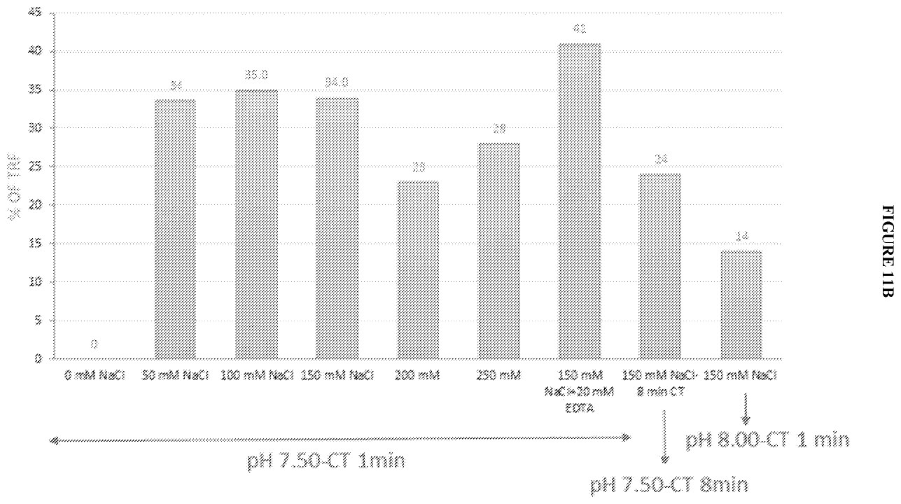

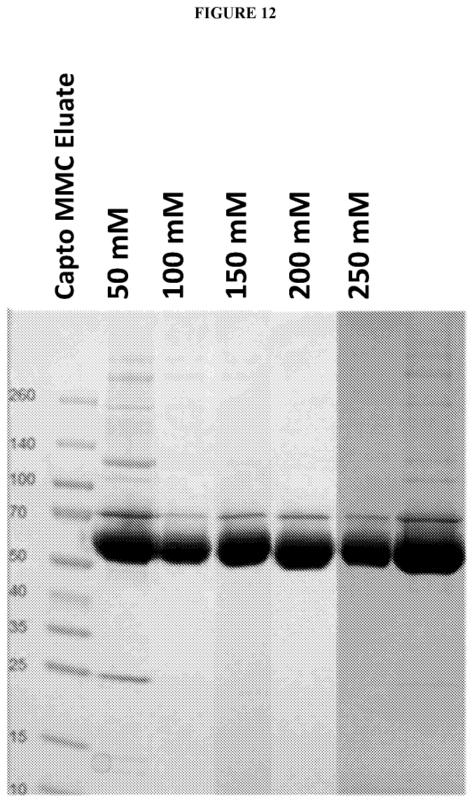

Very high hemopexin recovery was observed (92 to 97%), at loading NaCl concentrations between 100 and 200 mM NaCl, while highest removal of contaminant proteins (mainly transferrin) was achieved at 150 mM with 8 minute contact time and 200 mM with 1 minute contact time (FIG. 11). These results were reinforced by the SDS-PAGE analysis, which demonstrated very high hemopexin purity between 100 and 200 mM NaCl (FIG. 12).

The elution step for the previous process step, Capto MMC chromatography, utilised buffer with 150 mM NaCl and pH 7.5, well within the optimal conditions for Capto Adhere as described above. The process could therefore be streamlined if the same conditions were used for the Capto Adhere load. For this reason, 150 mM NaCl and pH 7.5 was selected as the most appropriate loading condition for Capto Adhere.

Binding capacity of the Capto Adhere resin was determined under the loading conditions determined above, using partially purified hemopexin (Capto MMC eluate), and a contact time of 7.5 minutes (120 cm/hr for column of 15 cm bed height). A hemopexin load of 250 mg per mL resin was applied to the resin, the equivalent of approximately 60 mg of contaminating protein per mL of resin. The breakthrough of haptoglobin, albumin and transferrin was measured in unbound fractions by immunonephelometry, and purity assessed by SDS-PAGE.

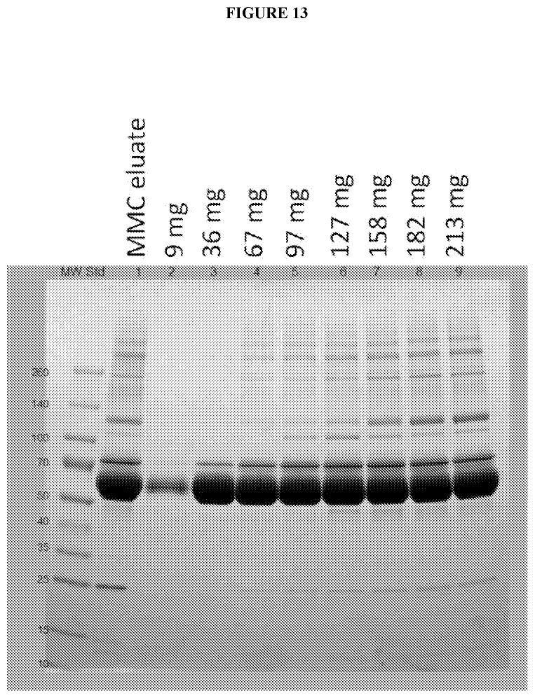

Analysis indicated that haptoglobin began to appear in unbound fractions after a hemopexin load of 36 mg hemopexin per mL resin. This was supported by SDS-PAGE analysis which demonstrated excellent hemopexin purity in fractions up to this load amount (FIG. 13). For simplicity, and to provide a margin for error, it was decided that an appropriate load limit for Capto Adhere chromatography be set to 30 g hemopexin/L resin. Break through of transferrin during Capto Adhere chromatography was almost immediate, meaning that any residual levels of transferrin in the product after Capto Adhere chromatography would require an additional purification step.

### Loading Conditions

To determine the robustness of Capto Adhere loading conditions, Capto MMC eluate was pH and conductivity adjusted and loaded onto Capto Adhere at a range of loading conditions; pH 7.0 to pH 8.0 and NaCl concentration 100 to 200 mM.

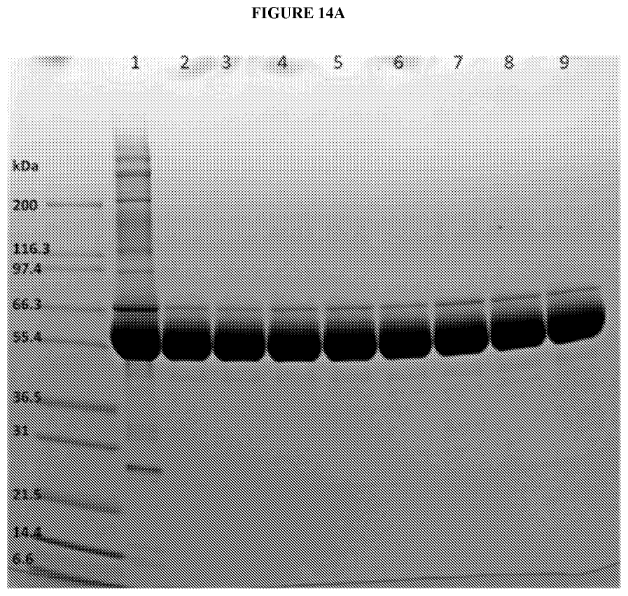

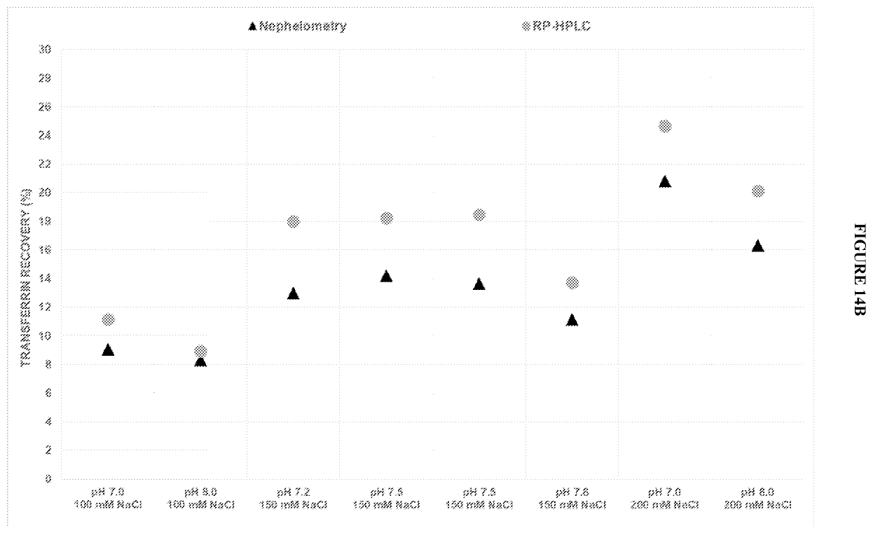

Hemopexin recovery over the Capto Adhere column was comparable for all loading conditions and almost complete removal of albumin and haptoglobin was observed by SDS-PAGE (FIG. 14). Transferrin recovery ranged from 9 to 25% when measured by RP-HPLC, with higher recovery observed at the 200 mM NaCl concentration level, indicating the higher conductivity is reducing the binding of contaminants (FIG. 14). These data indicate that optimal loading conditions for the Capto Adhere chromatography step are between pH 7.0 and 8.0 with NaCl concentration of 100 mM to 150 mM (approximately 15.8-21 mS/cm) to ensure maximum removal of transferrin. However, given the demonstrated ability of an additional cation exchange step to remove transferrin (Example 7), it may be feasible to extend the NaCl (conductivity) range to 200 mM (approximately 25.6 mS/cm).

During the manufacture of laboratory and pilot scale batches, the Capto MMC eluate was adjusted from pH 7.2-7.3 to pH 7.5 and from approximately 18.5 mS/cm to 20 mS/cm prior to loading onto the Capto Adhere resin. The Capto Adhere robustness study indicated that pH and conductivity of unadjusted MMC eluate would not impact the purification ability of the Capto Adhere resin or the recovery of hemopexin. The Capto MMC eluate could therefore be loaded without pH and conductivity adjustment to simplify the process in a manufacturing setting. A small scale run demonstrated that this was indeed the case, with no impact to chromatography profile, purity and hemopexin recovery.

A further processing run was performed to assess the feasibility of operating the Capto MMC and Capto Adhere chromatography columns in line. In contrast to a pooled Capto MMC eluate, the in line processing run demonstrated higher contaminant protein level by non-reduced SDS-PAGE gel, with a more intense transferrin band and haptoglobin bands visible. Lower hemopexin recovery was observed for the in line processing run with 33% hemopexin recovered from the clarified extract paste, compared to 47% when operated under current conditions. Operation of the Capto MMC and Capto Adhere columns in line was not considered to be suitable for use in a manufacturing process under the current conditions, but may be feasible with further optimization.

### Capto Adhere Chromatography Process Step

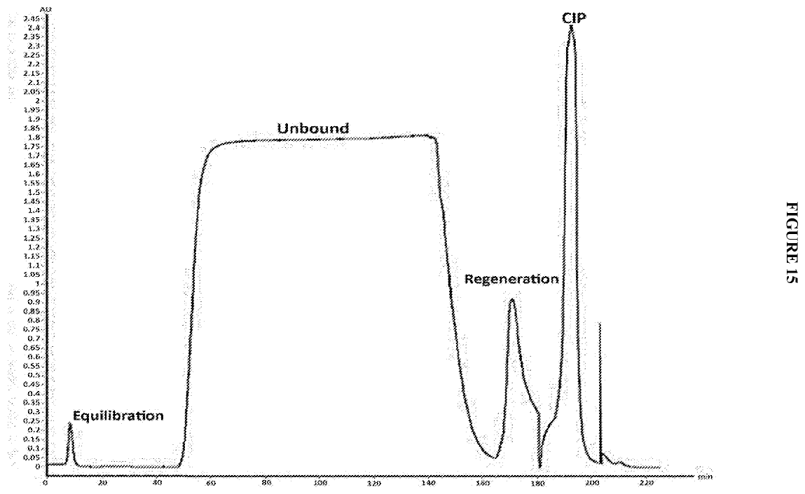

The Capto MMC eluate solution obtained from the previous step is typically at pH 7.2 with conductivity approximately 18 to 19 mS/cm. Although this differs slightly from the equilibration conditions for Capto Adhere, pH and conductivity adjustment of the Capto MMC eluate is not necessary. The product is loaded onto a Capto Adhere mixed mode chromatography column, equilibrated with 5 CV of 40 mM sodium phosphate, 150 mM NaCl pH 7.5. The product is loaded to a target load of 30 g hemopexin/L of resin and the unbound hemopexin fraction is collected along with a further 3 CVs wash of equilibration buffer. An example chromatography profile is shown in FIG. 15. The eluted hemopexin is typically >90% pure and is at a concentration of between 1 and 3 mg/mL. Following product collection, the Capto Adhere column is regenerated with 40 mM phosphate, 1M NaCl pH 7.5 buffer.

### Example 6—Virus Inactivation by Solvent Detergent Treatment

### Confirmation of Solvent Detergent Virus Inactivation

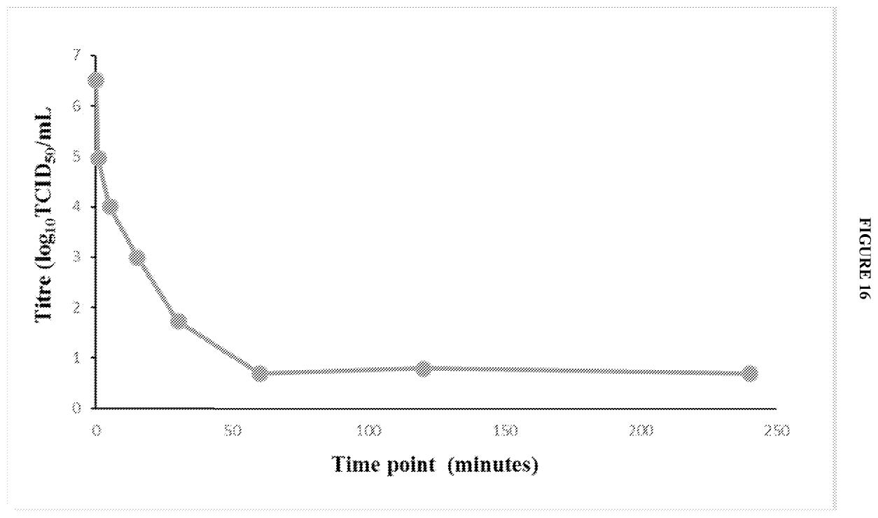

Solvent detergent (SD) treatment with 1% polysorbate 80 (PS80) and 0.3% tri-n-butyl phosphate (TnBP) represents one of the two dedicated virus removal/inactivation steps for the hemopexin process. This treatment disrupts the membrane of enveloped viruses and is a well-defined, robust method for virus inactivation in the blood plasma industry. The effectiveness of SD treatment for hemopexin was demonstrated in Capto MMC eluate using the Pseudorabies virus (PRV) as a model. In this study a viral titre reduction of approximately 5.7 log₁₀ was achieved after 60 minutes incubation without compromising the hemopexin intermediate (FIG. 16).

A stability study with Capto MMC purified hemopexin maintained in SD solution for 24 hours at 25° C. showed no change to product purity or aggregation state, and negligible changes to heme binding activity. This, in conjunction with the clearance data, shows that SD treatment is a viable strategy for virus reduction in the hemopexin process.

If SD treatment is performed with Capto MMC eluate, however, (as in the spiking/clearance study), the likelihood exists of SD interference with the subsequent Capto Adhere purification step. In addition, it is unlikely that SD will be removed by this step. To avoid these issues, SD treatment was performed on material after Capto Adhere treatment. As protein purity is increased following Capto Adhere, it is unlikely that viral inactivation will be adversely affected by this change. This will also limit the number of purification steps required to be performed in post-VI grade areas during commercial manufacture.

### Solvent Detergent Treatment Process Step

Capto Adhere eluate is filtered through a 0.22 μm filter into a jacketed vessel and heated to 21 to 25° C. A stock solvent and detergent solution comprising of 20% w/w polysorbate 80 (PS80) and 6% w/w Tri-n-Butyl Phosphate (TnBP) is added slowly to the Capto Adhere eluate over 5 to 10 minutes to a target concentration of 1.0% w/w PS80 and 0.3% w/w TnBP. The solvent detergent treated bulk is stirred using a stirring rate and impeller size such that vortexing occurs to about approximately 10% of the vessel depth, without causing aeration. After at least 15 minutes the stirring rate is reduced such that vortexing occurs to about 5% of the vessel depth, without causing aeration. Incubation commences once the solution temperature reaches 23° C. and is performed for 2 to 24 hours with stirring, and temperature maintained at 23±2° C.

### Example 7—Ion Exchange Chromatography with Eshmuno CPS Resin

Four ion exchange chromatography resins, Capto Q, Capto DEAE, Capto S and Eshmuno CPS were investigated for removal of the solvent detergent process reagents from hemopexin process intermediate. The Capto Q chromatography resin, which is used in an earlier hemopexin process (as previously described in WO2014/055552), was demonstrated to bind hemopexin and contaminant proteins, such as transferrin, haptoglobin and albumin. Elution of hemopexin was observed at NaCl concentrations from 50 to 100 mM, however, transferrin was demonstrated to co-elute with hemopexin. Further investigation over the pH range pH 7.0 to 8.0 demonstrated no additional reduction in transferrin contamination.

Capto DEAE chromatography resin was observed to bind hemopexin and the contaminant proteins, however a small loss of hemopexin of approximately 5% was observed in the drop through fraction. Transferrin was observed to co-elute with hemopexin over a range of NaCl concentrations, with haptoglobin eluting at ≥150 mM NaCl and albumin at ≥200 mM NaCl. Capto S chromatography resin was demonstrated to not be suitable to bind hemopexin, with hemopexin present in the drop through fraction.

Chromatography resin screening studies identified Eshmuno CPS ion exchange resin as a suitable resin for use in a chromatography polishing step following the solvent detergent treatment process step (Example 1). The Eshmuno CPS resin demonstrated high binding capacity for hemopexin, with efficient removal of transferrin and demonstrated superior purification to the Capto Q, Capto DEAE and Capto S resins.

### Development and Optimisation of Eshmuno CPS Chromatography

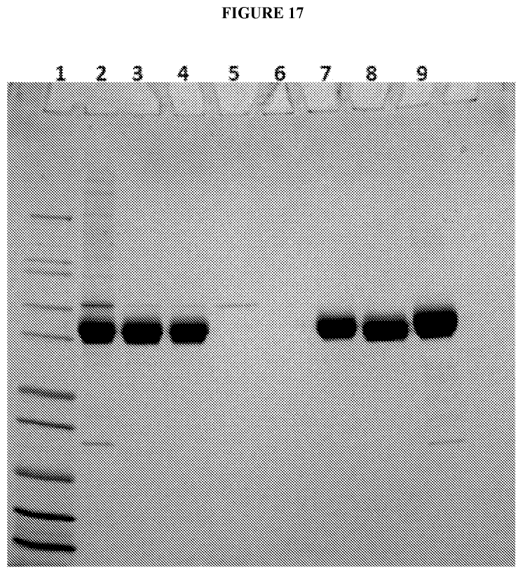

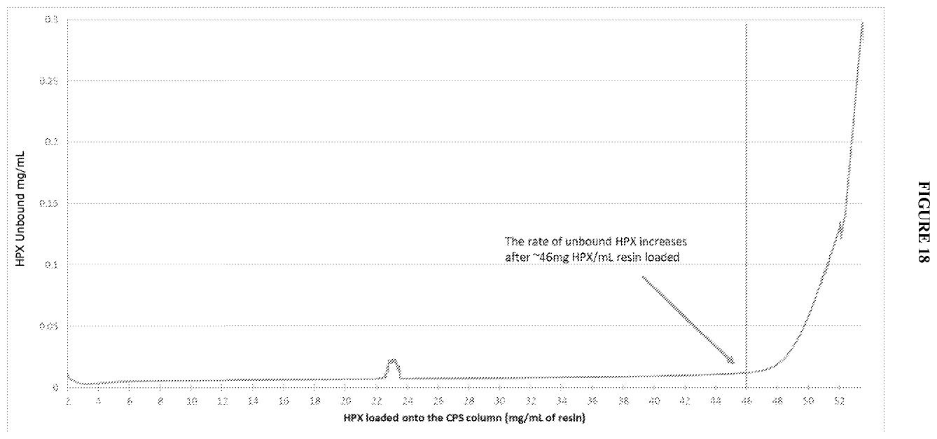

Initial evaluation of the Eshmuno CPS resin with SD treated Capto Adhere eluate was performed with equilibration and load conditions at pH 6.0 and a conductivity of 10 mS/cm (Example 1). Elution of bound hemopexin was performed using a step gradient with 100 mM, 200 mM and 300 mM NaCl. Under these conditions, purification of hemopexin was demonstrated with 99% of transferrin recovered in the drop through fraction while 98% of hemopexin was recovered with elution at 300 mM NaCl. The final purity of hemopexin after Eshmuno CPS chromatography is shown in the SDS-PAGE analysis (FIG. 17).

To simplify the operation of the hemopexin process, elution of hemopexin from the Eshmuno CPS resin in the hemopexin formulation buffer would provide the advantage of reduced diafiltration time during the UF/DF and reduced number of buffers required. A small scale processing run resulted in the elution of 99.6% of loaded hemopexin with formulation buffer (0.9 mM citric acid, 14.1 mM sodium phosphate, 150 mM NaCl pH 7.2) demonstrating the feasibility of eluting directly into the hemopexin formulation buffer. In later studies on viral filtration, it was found that a NaCl concentration of 0.6 M enabled greater filter throughput (Example 8). Given that all contaminating protein is found in the unbound fraction in Eshmuno CPS chromatography, it is likely that elution can be performed in this NaCl concentration to enable a simpler transition to the virus filtration step.

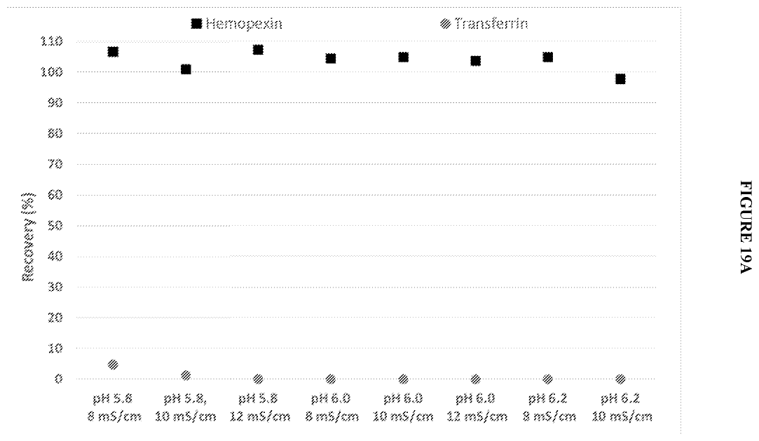

Binding capacity for the Eshmuno CPS resin was determined using Capto Adhere eluate as the feedstock, and loading the column to the point of breakthrough. Binding conditions were at pH 6.0, 10 mS/cm as described above. Breakthrough was observed after loading 46 g hemopexin per L resin (FIG. 18). As a result, the target load for hemopexin on the Eshmuno CPS resin was set at approximately 90% of the break through point, 40 g hemopexin/L resin, to minimise the risk of overloading and loss of hemopexin over the process step.

The ability of Eshmuno CPS resin to remove polysorbate 80 was demonstrated by loading 1% polysorbate 80 as a sample. Analysis of the drop through, elution and regeneration fractions demonstrated 100% recovery of the loaded polysorbate 80 in the drop through fraction, demonstrating that polysorbate 80 is not retained by the Eshmuno CPS resin.

### Loading Conditions

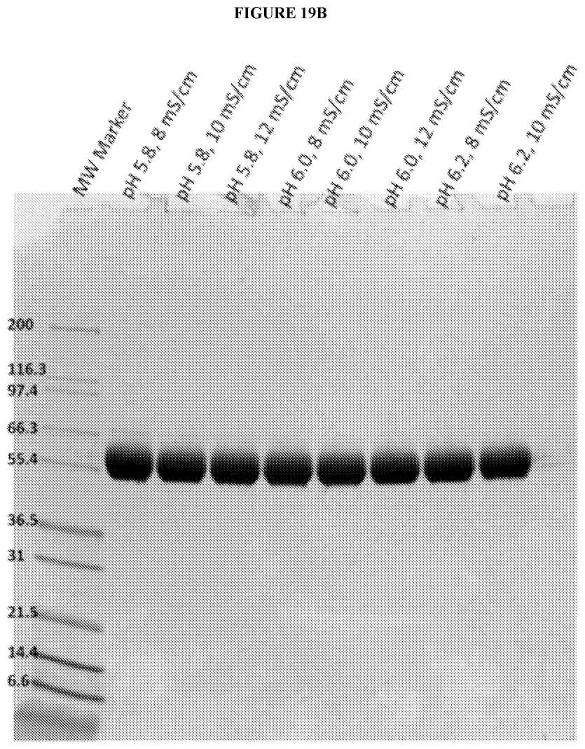

To determine robustness of the loading conditions, solvent detergent treated Capto Adhere eluate was spiked with transferrin and loaded onto Eshmuno CPS columns at a pH of between 5.8 to 6.2 and a conductivity 8 to 12 mS/cm. It was observed that transferrin removal over the Eshmuno CPS column was optimal with loading conditions at pH 6.0 to 6.2, and a conductivity of 10 to 12 mS/cm, but with acceptable results at 8 mS/cm. At the pH 5.8 loading condition, transferrin was detected by RP-HPLC in the Eshmuno CPS eluate at the 8 and 10 mS/cm conductivity levels (FIG. 19). The recommended loading conditions for the Eshmuno CPS chromatography step are therefore pH 6.0 to 6.2 with conductivity 8.0 to 12 mS/cm.

### Eshmuno CPS Chromatography Process Step

Solvent/detergent treated Capto Adhere eluate is pH adjusted to pH 6.0±0.1 with 0.5 M acetic acid and diluted with PFW or WFI to a target conductivity of 10 mS/cm. The adjusted product is loaded onto an Eshmuno CPS ion exchange chromatography column, equilibrated with 5 CV 25 mM sodium phosphate, 25 mM sodium acetate, 38 mM NaCl, pH 6.0. The adjusted product is loaded to a target load of 40 g hemopexin/L of resin. After product loading, the column is further washed with 3 CVs of equilibration buffer to remove SD and unbound proteins. Hemopexin is eluted with 3 CVs of 20 mM sodium phosphate, 0.6 M NaCl, pH 7.2. Collection of Eshmuno CPS eluate commences when the UV absorbance is ≥50 mAU (2 mm path length) and continues until the UV absorbance returns to baseline (A₂₈₀ₙₘ≤50 mAU for 2 mm path length). The eluted hemopexin is typically >95% pure and is at a concentration of between 6 and 10 mg/mL. Following product collection, the Eshmuno CPS column is regenerated with 40 mM phosphate, 1M NaCl pH 7.5 buffer.

### Example 8—Optimisation of Virus Filtration for the Hemopexin Process

Virus filtration represents one of the two dedicated virus removal/inactivation steps in the hemopexin process. The virus filtration step removes viruses based on size differences, and is a well-defined, robust method for virus reduction in the blood plasma industry. The virus filtration method from the first generation hemopexin process, using the Planova BioEX virus filter, as previously described in WO2014/055552) laid the foundation for further development of the virus filtration step. Literature shows that various factors can affect nanofiltration, including pressure, protein concentration, conductivity and pH of the solution and the effect of each of these factors on virus filtration throughput was investigated. Development studies performed on the virus filtration step are summarised below.

### Development and Optimisation of Virus Filtration

Initial studies indicated that higher pressure resulted in greater filter throughput, with 152 L/m² observed at 1.5 hours at 3 bar compared to 113 L/m² at 2 bar. However, a higher decay rate was also observed for filtration at 3 bar. When the data was fitted to an exponential decay model it showed that maximum throughput was higher at the lower pressure of 2 bar, with 473 L/m² compared to 371 L/m² at 3.0 bar, in contrast with the throughput data at 1.5 hours. Lower throughput at 3 bar is consistent with the ‘trapping’ theory of particle retention in virus filters, where particles are retained in pockets under high pressure, but allowed to diffuse out of these pockets under lower pressure and it is for this reason that higher pressures are preferred for viral clearance. Although the results of this study indicate that throughput at 3 bar is lower than that at 2 bar, the throughput achieved was acceptable for commercial manufacture of hemopexin. This, in combination with the time saving achieved with 3 bar, and the more preferable pressure for viral clearance, led to the decision to utilise 3 bar for all subsequent studies.

Development studies to optimise virus filtration throughput were performed around the pH and conductivity of the Eshmuno CPS eluate using a Sartopore 2 XLM pre-filter coupled to a BioEX filter.

No substantial difference in the filtration throughput and flux decay was observed with Eshmuno CPS eluate over a pH range of 6.5 to 7.5. Based on this finding it was decided that adjustment of the eluate pH prior to virus filtration would offer little or no throughput advantage.

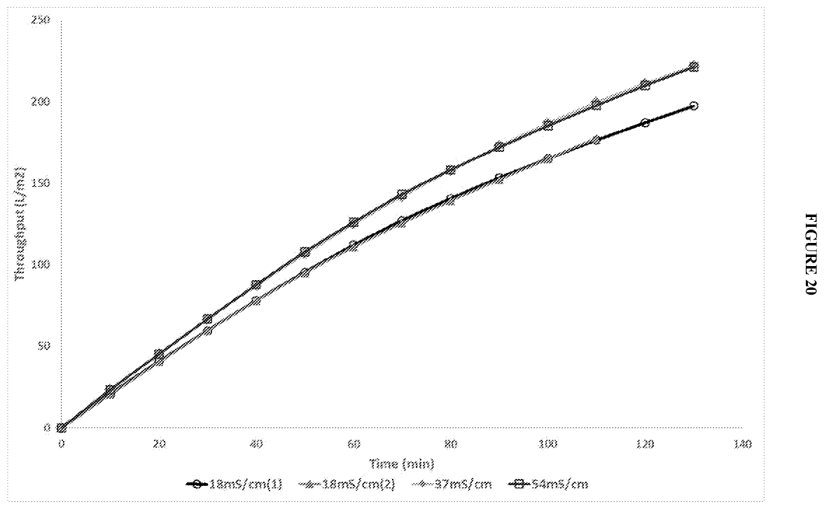

For the initial hemopexin process, as previously described in WO2014/055552, the conductivity of the pure hemopexin material for virus filtration was equivalent to 600 mM NaCl. Early development studies for the second generation process also showed superior throughput at high conductivity. Confirmation studies identified that moderate to high conductivity resulted in increased throughput, with CPS eluate at conductivity values of 37 mS/cm (600 mM NaCl) and 54 mS/cm (1 M NaCl) demonstrating equivalent throughput, 13% greater than 18 mS/cm (150 mM NaCl) (FIG. 20). Flux decay rate did not vary significantly with conductivity, despite the change in throughput, indicating that increased conductivity may not increase the permeability of the filter, or reduce the fouling rate, but rather allows the solution to access a greater available area, or a greater number of pores.

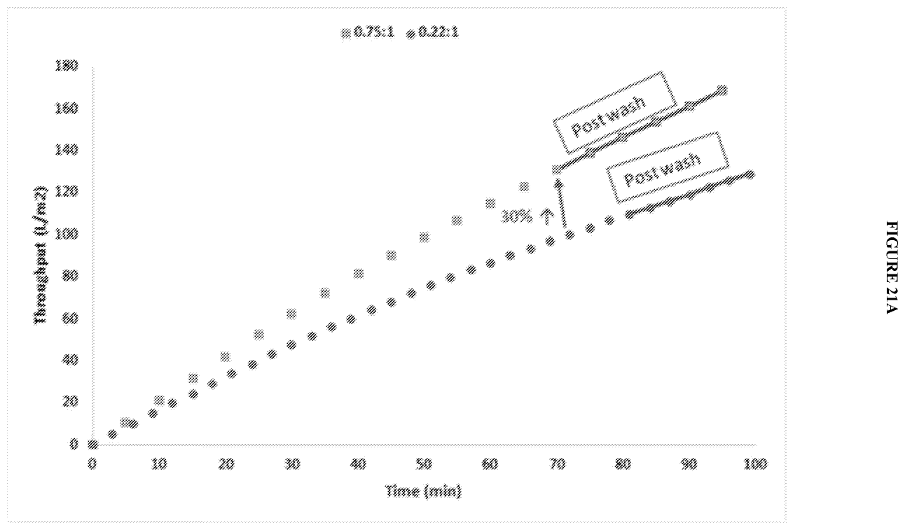

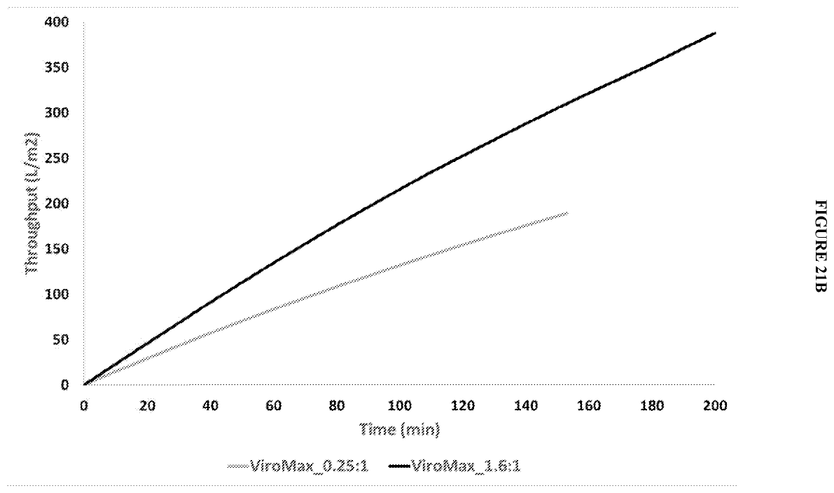

During pilot scale manufacture, low virus filtration throughput was observed, with the filters fouling after only 45-70 L/m². When the ratio of pre-filter to virus filter surface area was investigated as a possible cause, throughput was shown to be significantly improved when a larger pre-filter area was employed. In this experiment, a pre-filter area: virus filer area ratio of 0.75:1 led to about a 30% increase in throughput compared to a 0.22:1 ratio (FIG. 21A). Flux decay was also lower with the higher pre-filter area, indicating that with higher loading, greater differences may become apparent. To account for differences in flux decay and limitations in sample volume, the data from subsequent experiments were fitted to an exponential curve and extrapolated to determine theoretical maximum throughput.

When the above experiment was repeated with a different pre-filter, Virosart Max (Sartorius), throughput was improved further (FIG. 21B). Curve fitting of the filtration data predicted that a maximum throughput of 551 L/m² could be achieved with this prefilter, at a 0.25:1 filter area: virus filter area ratio, rising to 1040 L/m² with a 1.67:1 ratio. This is clearly superior to the maximum throughput obtained with Sartopore XLM, and would allow the use of a smaller Virosart Max prefilter.

Assessment of alternative virus filters as replacement options for the Asahi Kasei BioEX identified the Sartorius Stedim Virosart HF filter as a suitable replacement, with a maximum throughput of 1068 L/m², slightly higher than 1044 L/m² for the BioEX in the same experiment. The Sartorius Stedim Virosart HC and Merck-Millipore Viresolve Pro filters were not suitable alternatives as both had lower maximum throughput than the BioEx filter. As BioEX has undergone early virus validation studies for use in the hemopexin process, and was utilised in the production of material for the hemopexin Phase 1 study referred to elsewhere herein (and as described in WO2014/055552), it remains the preferred filter. However, the throughput of the Virosart HF filter highlights its potential use as a replacement for the BioEX filter, if required.

### Virus Spiking Experiments

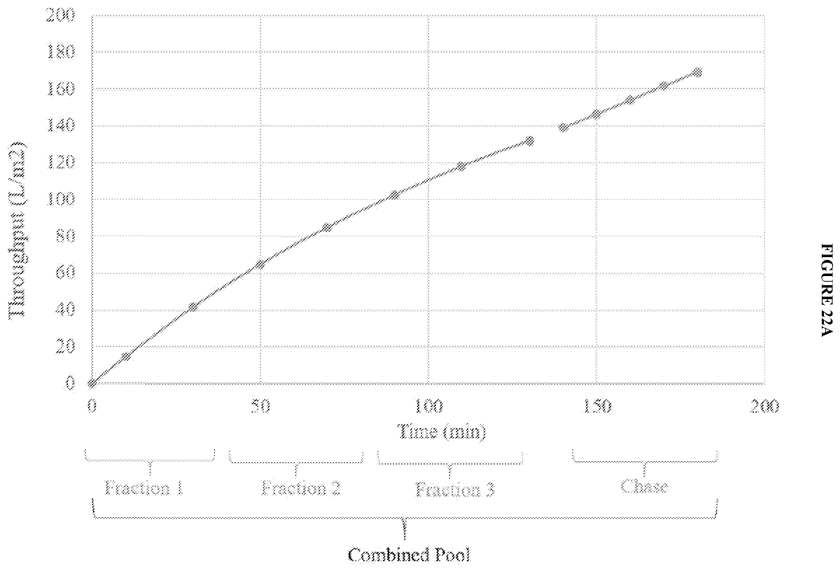

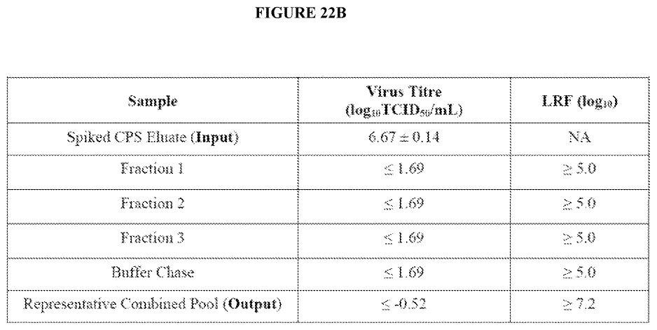

The effectiveness of the Asahi BioEX filtration step was assessed by its capability to remove the model virus MVM (Minute Virus of Mice) from purified hemopexin. MVM, a worst-case model virus for filtration due to its small size, was spiked into Eshmuno CPS eluate, and filtered with an Asahi BioEx filter with 0.0003 m² filter area. Excellent viral clearance was achieved throughout the filtration process via evaluation of the associated sample fractions under worst-case combined conditions for virus breakthrough. A final reduction in viral titre of ≥7.2 log₁₀ was also achieved for the representative combined pool (FIG. 22). This demonstrates that the BioEX filter provides adequate virus clearance for implementation in the hemopexin process.

### Virus Filtration Process Step

The Eshmuno CPS eluate is filtered through a 0.1 μm pre-filter in series with a Planova BioEX filter at a pressure of 3.0±0.1 bar with a filter area of 1 m²/100 L Eshmuno CPS eluate. The prefilter area should be at least 0.6 times the virus filter area. The filters are post-washed with three BioEX capsule volumes of virus filter wash buffer, which is pooled with bulk filtrate to form the BioEX filtrate.

### Example 9—Concentration and Diafiltration of BioEX Filtrate

The concentration and diafiltration process step for the initial hemopexin process, as previously described in WO2014/055552) was used for the current process without modification. Implementation of the concentration and diafiltration process step into the hemopexin process was successful and no further development or optimisation of the process step was performed.

### Example 10—Identification and Quantification of Heme-Hemopexin Complex in FIV-4 Paste

During development of the Capto MMC chromatography process step, poor hemopexin recovery was observed, with a significant portion of hemopexin observed in unbound fractions. This seemed to vary depending on the batch of paste used. The fact that this occurred at low column loads and optimal binding conditions (Example 6), led to the hypothesis of the presence of a population of hemopexin with different physicochemical characteristics. Early studies showing a lack of binding to heme agarose and literature evaluation suggested that this population may be composed of heme-hemopexin complex.

In a key study, a 50% mixture of hemopexin and heme-hemopexin complex was applied to Capto MMC under the conditions established above, with protein monitored by absorbance at 280 nm and heme-hemopexin complex monitored at 411 nm. The result clearly showed pure hemopexin eluting in the bound fraction, and heme-hemopexin complex eluting in the unbound fraction (FIG. 23).

Whilst a lack of binding of heme-hemopexin complex to Capto MMC had been shown, the presence of complex in extracted paste had not been demonstrated. To address this, heme-hemopexin complex was isolated from the unbound fraction from Capto MMC purification of extracted paste. Heme-hemopexin complex was found to not bind Capto Adhere under the process operating conditions used for hemopexin purification, allowing this step to be used to purify complex from the Capto MMC unbound fraction. In this experiment, with Capto Adhere loaded at a low volume, most of the contaminant proteins remained bound to the Capto Adhere resin and purified heme-hemopexin complex was collected in the drop through fraction. The resulting purified heme-hemopexin complex contained a significant amount of transferrin by SDS-PAGE, however, the absorbance spectrum showed a peak at 414 nm for the heme-hemopexin complex without a peak at 475 nm for holo-transferrin. This clearly demonstrates the presence of heme-hemopexin complex in the Capto MMC unbound fraction.

This result indicated that the amount of heme-hemopexin complex could be estimated using Capto Adhere Impres chromatography with small injection volumes. Under these conditions, hemopexin and heme-hemopexin complex are eluted as a single broad peak in the unbound fraction, with all other proteins retained. As the extinction coefficients for hemopexin at 280 nm and heme-hemopexin complex at 414 nm are almost identical, the proportion of heme-hemopexin complex can be estimated by the ratio of 414 nm peak area to that at 280 nm.

Initial analysis indicated that approximately 15% of hemopexin in the clarified extracted paste was present as heme-hemopexin complex, although this has been found to vary depending on the batch of paste.

A second assay to measure the heme-hemopexin complex content was developed, using a combination of RP-HPLC and Size Exclusion HPLC (SE-HPLC) with detection at 412 nm. Size exclusion HPLC of extracted paste was able to identify a reasonably well resolved peak for heme-hemopexin complex that could be integrated and quantitated relative to a standard curve. Free hemopexin concentration was determined by RP-HPLC, and from the two data sets, the portion of hemopexin in complex with heme could be determined. The assay was used to quantify the proportion of heme-hemopexin complex in nine different batches of paste, and showed that complex amount varied considerably, from 3.7 to 17.2% of total hemopexin, depending on paste batch.

Taken together, the data from the above studies show that the amount of complex may vary considerably depending on the batch of paste. As heme-hemopexin complex is not recovered in the hemopexin purification process, a clear understanding of the amount of complex is required to fully understand process recovery.

### Example 11—Process Description

The final developed process involves the purification of hemopexin from FIV-4 paste, with three chromatography steps. After paste solubilisation and clarification, hemopexin is purified by Capto MMC and Capto Adhere chromatography, before being subjected to viral inactivation by solvent/detergent treatment. The product is then polished using an Eshmuno CPS salt-tolerant cation exchange column, and nanofiltered for virus removal before diafiltration into formulation buffer.

Whilst Examples 2 to 10 outline most of the development of each of these steps, some further optimisations and simplifications were made following large lab-scale and pilot scale purification campaigns.

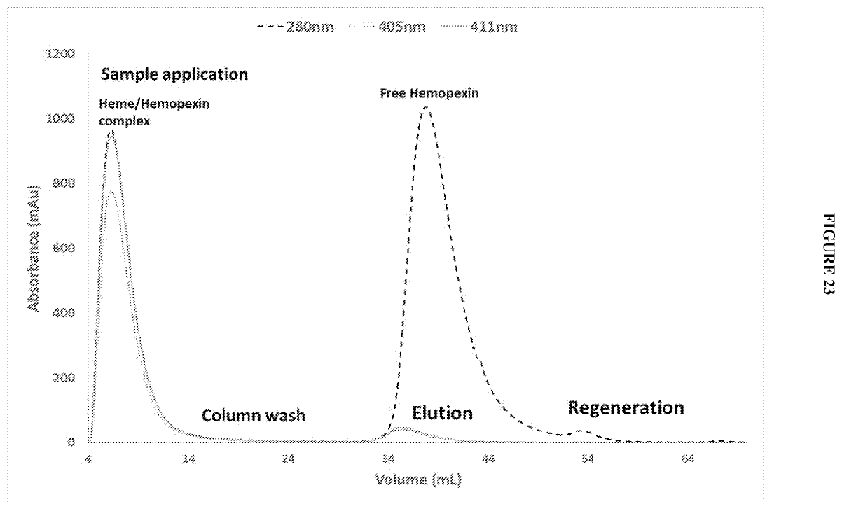

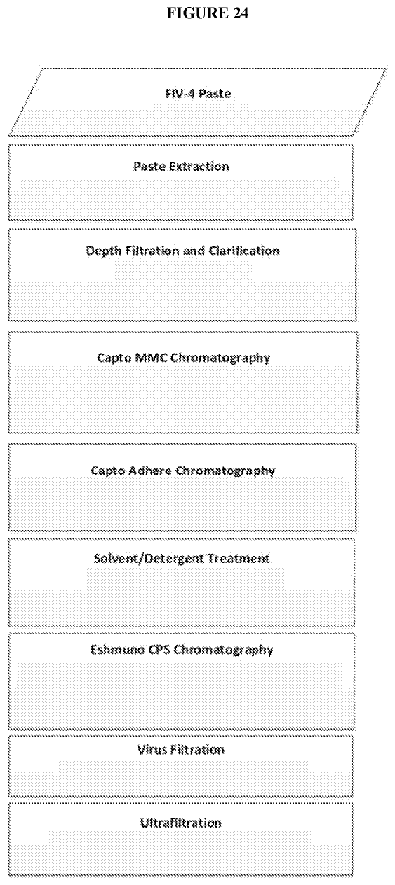

The final process including all optimisations is outlined in FIG. 24Error! Reference source not found.

### Example 12—Process Reproducibility

After initial development and optimization, the purification process was performed three times to assess reproducibility. Although this study was performed before all optimization and process streamlining was complete, most process parameters were established at this stage, and only small changes were made hereafter. The process was performed essentially as outlined in FIG. 24, but with extraction performed at a 1:10 paste: buffer ratio in 180 mM NaCl at pH 6.2 and with the Capto MMC column equilibrated in the same buffer.

Three batches were produced at small laboratory scale from 3 batches of paste-Batches 1, 2 and 3. Batches 1 and 2 were derived from batches adsorbed for Berinert/Beriplex production. Process intermediates and the final purified product were assayed for hemopexin concentration by immunonephelometry and RP-HPLC and for purity by SDS-PAGE. Final purified product was further analysed for activity by heme binding.

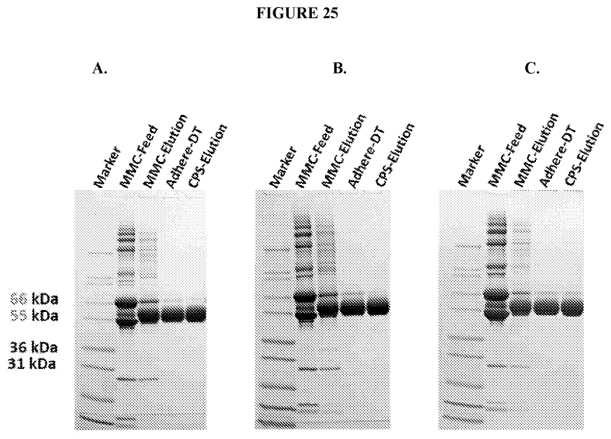

The results show that the process was quite reproducible regardless of the paste batch used, with hemopexin purity and specific activity almost identical across the three batches (Table 3 and FIG. 25). In all cases, purity was very high, at over 99% by SDS-PAGE and densitometry, and heme binding activity ranging from 99.6 to 104.9% of total protein. Whilst there is some variation in these activity numbers, they remain extremely high, and are likely reflective of assay variability rather than process or batch inconsistencies.

One possible cause of variation in yield and recovery is the amount of heme-hemopexin complex present in the starting material. Hemopexin in this complex is included in quantitation of the starting material, but the complex is removed during the Capto MMC chromatography step of the process. As the amount of complex varies between batches of paste, this can lead to variations in yield and recovery between batches.

When recovery over each processing step is compared, it is apparent that most of the process losses occur during the Capto MMC chromatography step. This is to be expected to some extent, due to the removal of heme/hemopexin complex during this step. Despite this, a reasonably consistent recovery was seen over this step of between 61 and 71%. Recovery at all other steps was high and consistent.

Taken together, the data show that the process yields and recoveries are reasonably consistent across several batches of paste. Product quality was very high and consistent in all cases, and no notable differences were apparent with paste subjected to Beriplex/Berinert adsorption.

### Example 13—Large Laboratory Scale Manufacture

Laboratory scale batch manufacture was performed during the development of the hemopexin process to provide baseline data and demonstrate the feasibility of the process. Four laboratory scale batches were manufactured at 3 kg FIV-4 paste scale and processed through to hemopexin drug substance. A number of changes to the hemopexin process and optimisation of process conditions were implemented over these batches.

### Initial Laboratory Scale Batches

The initial laboratory scale batches were performed with a 1:10 FIV-4 paste to buffer extraction ratio. The extract was clarified by depth filtration and purified via chromatography using Capto MMC and Capto Adhere resins. Following the two initial chromatography steps, the product was solvent detergent treated for viral inactivation, and the solvent detergent reagents removed via chromatography using Capto Q resin. The resulting process intermediate was subjected to virus filtration using a BioEX filter (laboratory scale batch 2 only) prior to UF/DF into formulation buffer to give hemopexin DS.

The two laboratory scale batches were successfully manufactured up to drug substance stage.

Overall, the quality of hemopexin purified by these initial laboratory scale batches was quite high with heme specific activity >90%, hemopexin monomer content >95%, and contaminant proteins reduced to below detectable levels (Table 4). The quality of the final product was also quite consistent between the two batches.

### Optimised Laboratory Scale Batch

After optimisation of several process conditions, a further batch was manufactured to evaluate the complete process at laboratory scale. Conditions were optimised for the FIV-4 extraction step (Example 4) and the filter frame depth was increased to 4 cm to allow for higher solid loads. Loading conditions were improved for the Capto MMC chromatography step (Example 4), the Capto Q chromatography step was replaced with Eshmuno CPS (Example 7), and various buffer conditions were optimised. The changed process conditions are outlined in Table 5.

The optimised laboratory scale batch was successfully manufactured up to drug substance stage, with a hemopexin recovery of 73% achieved across the process. As a different batch of FIV-4 paste was used in this batch, the improvement in recovery over previous batches may be due to differences in the amount of hemopexin and heme/hemopexin complex present. Recovery may also have been improved with more efficient recovery from the clarification step, through the use of a more appropriate frame size.

The hemopexin drug substance produced had a heme binding specific activity of 97%, with monomer content of >99% and the contaminant proteins albumin, transferrin and haptoglobin reduced to undetectable levels (Table 6). Overall, the optimised laboratory scale batch demonstrated that the changed conditions resulted in good process recovery, and a highly pure hemopexin product.

### Streamlined Laboratory Scale Batch

Following pilot scale batch manufacture (Example 14), further optimisation was performed to streamline the process at scale. An increase in extraction buffer NaCl concentration removed the need for conductivity adjustment prior to Capto MMC chromatography (Example 2). It was also found that Capto Adhere chromatography could be performed without pH and conductivity adjustment of the Capto MMC eluate (Example 5).

A streamlined laboratory scale batch was manufactured to confirm that the changes in process did not affect the hemopexin purity and yield. The laboratory scale batch was manufactured up to the Eshmuno CPS eluate stage, with hemopexin eluted from this column at a lower NaCl concentration to provide material for virus filtration conductivity studies. The changed process conditions are outlined in Table 7.

A total process recovery of 55% and a yield of 0.18 g/LPEQ were observed for the streamlined batch, which is low, but consistent with yields and recoveries of previous batches. As a different batch of paste was used in this study, again, the amount of hemopexin and heme/hemopexin complex may have played a role. On the whole, it is considered unlikely that the streamlined conditions led to decreased recovery.

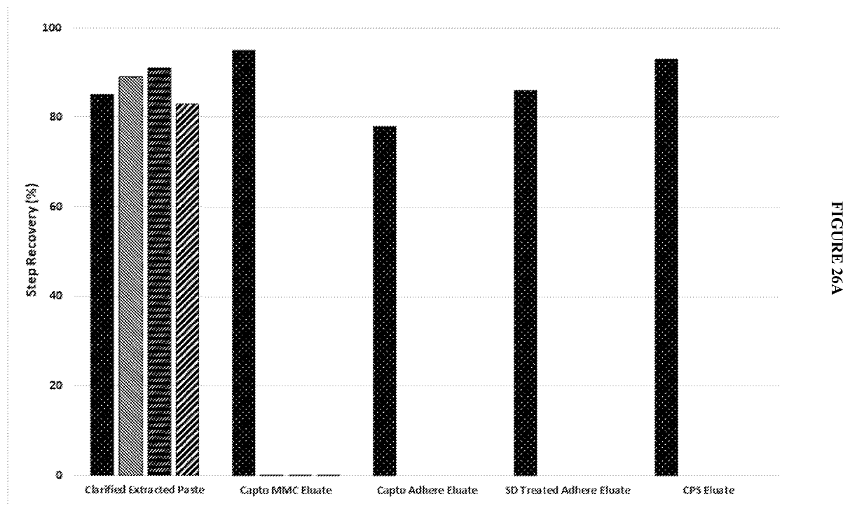

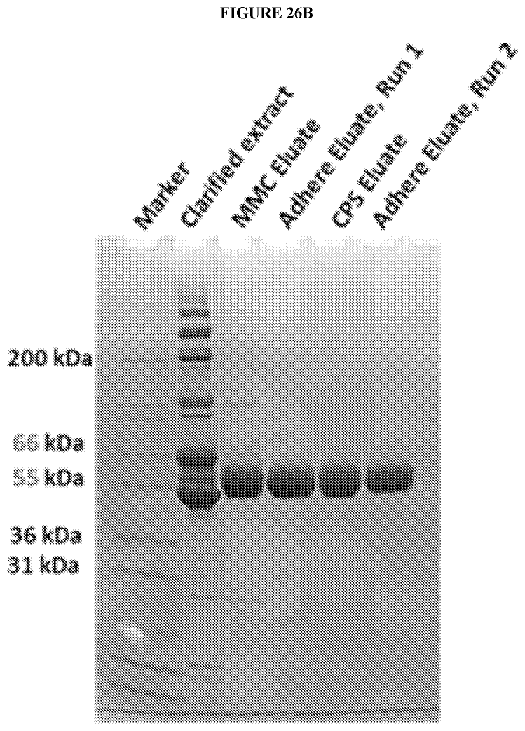

By SDS-PAGE analysis, hemopexin purity was quite high following Capto MMC chromatography, with no visible contaminant bands after subsequent chromatography steps (FIG. 26). Very high purity was achieved following Eshmuno CPS chromatography, consistent with previous laboratory scale batches.

### Example 14—Pilot Scale Batch Manufacture

Pilot scale batches were conducted to demonstrate that the process was scalable and capable of producing consistent product intermediates and hemopexin drug substance. In addition, these pilot scale batches helped to identify potential process issues that may occur at large scale manufacture.

Two pilot scale batches were manufactured at 20 kg FIV-4 paste scale, with the same batch of FIV-4 paste used as the starting material. The process was performed following the parameters outlined in FIG. 44, with the following exceptions: paste was thawed at 4° C. for 2.5 days before extraction with buffer containing 225 mM NaCl at pH 6.4. Due to limitations in maximum pump speed, the extract was divided in half and clarified using a filter area appropriate for half the paste mass, before recombining the filtrates. Due to limited availability of column hardware, the Capto Adhere and Eshmuno CPS columns were not large enough to accommodate all of the Capto MMC eluate. As the Capto MMC column was loaded to capacity, this meant that only around 65% of the Capto MMC eluate was carried through the rest of the process, with the remainder discarded. Whilst wasteful, this allowed assessment of product quality with all chromatography steps at capacity.

Virus filtration throughput was surprisingly low in both pilot scale batches, with total throughputs of only 47 and 70 L/m² for batch 1 and 2 respectively. This led to further development work around filtration conditions, as discussed above in Example 10. Following on from this development work, two further large scale batches were performed under similar conditions to pilot scale, and the material used to verify virus filter throughput with the optimised conditions. In essence these conditions included the use of a 650 cm² Sartopore XLM pre-filter with a 0.1 m² BioEx virus filter, and feed conditions with 37 mS/cm conductivity. Under these circumstances, all available material was easily filtered with only moderate decay. Maximum throughput was calculated at 521 L/m² for the first of these batches, and 232 L/m² in the second. Whilst it remains unclear as to why the two throughputs are different, they are clearly a substantial improvement over that seen in the first two pilot scale batches.

### Intermediate and Process Recoveries at Pilot Scale

When comparing the two pilot scale batches, comparable recoveries were observed for all steps, with the exception of Capto Adhere chromatography (Table 11). It is suspected that the low recovery seen over this step in Batch B is due to inadequate mixing of the product prior to sampling, leading to an inaccurate concentration measurement. A similar error is also apparent in the Eshmuno CPS recovery calculation, which returned a value of 130% in the same batch. Comparable overall process recoveries for the two batches are also incompatible with such variations in step recovery.

Over the depth filtration and clarification step, approximately 80% recovery of hemopexin was observed for both batches with approximately 80% recovery of transferrin, albumin and haptoglobin observed for the first batch (Batch A) (Tables 12 and 13). This is consistent with a loss of product volume over this step, potentially due to inadequate filter press post-washing. In the second pilot scale batch, Batch B, variable recovery for the contaminant proteins was seen over the depth filtration and clarification step, ranging from 60 to 90%. In this case, sampling variability is suspected.

The majority of contaminating protein was removed in the Capto MMC step, with the recovery of around 85% of total hemopexin (Tables 11 and 12). At least some of the loss of hemopexin can be attributed to the presence of heme-hemopexin complex in the clarified extract, which is not expected to bind to Capto MMC (Example 10). Analysis of the clarified extract used in the pilot scale batches indicated that up to 20% of total hemopexin may be present as heme complex. This could easily account for the majority of hemopexin lost in this step.

Minimal protein or hemopexin loss was observed over the Eshmuno CPS chromatography, virus filtration, UF/DF and sterile filtration process steps, with approximately 100% recovery observed.

The overall process recovery of hemopexin was approximately 50 to 60%. As outlined above in Example 10, the FIV-4 paste contains a portion of hemopexin in complex with heme, which varies with paste batch. As heme-hemopexin complex is undesirable in the final product and is removed during the Capto MMC step of the process, an understanding of the recovery of free hemopexin is more appropriate. When the concentration of complex is taken into account, the recovery of free hemopexin was around 70% for both pilot scale batches, demonstrating a high recovery of usable product.

In summary, the data outlined above show that reasonably consistent recoveries were achieved over each step of the process, and over the process as a whole when performed at pilot manufacturing scale.

The process yielded 50 and 51 grams of purified hemopexin from the two pilot scale batches, derived from the equivalent of 13 kg of FIV-4 paste (corrected for material discarded before Capto Adhere). This equates to 0.15 grams of purified hemopexin per litre plasma input.

### Intermediate Product Quality at Pilot Scale

The purity of process intermediates was as expected for both pilot scale batches, with most contaminating protein removed at the Capto MMC stage and remaining transferrin almost completely removed in subsequent steps. The purity profile of each process intermediate was almost identical over the two batches.

Protease activity was quite different in the two batches of extracted paste (Table 8). It is suspected that the high value seen in Batch A is due to the presence of filter aid in the sample, leading to protease activation in the time between sampling and analysis. This sample in Batch B was filtered immediately after sampling, preventing further activation by this mechanism. Regardless, in both batches, protease activity became very low after clarification and remained low for the remainder of the process.

Prekallikrein activator was found to be at <20 IU/mL in extracted paste, and remained at this level throughout the process regardless of batch (Table 9).

Taken together these data show that the process yields hemopexin of consistent purity at each step. Protein contaminants, proteases and PKA are consistently removed, and are at very low levels in final drug substance, even at a hemopexin concentration of 100 mg/mL.

### Characterisation of Hemopexin Drug Substance

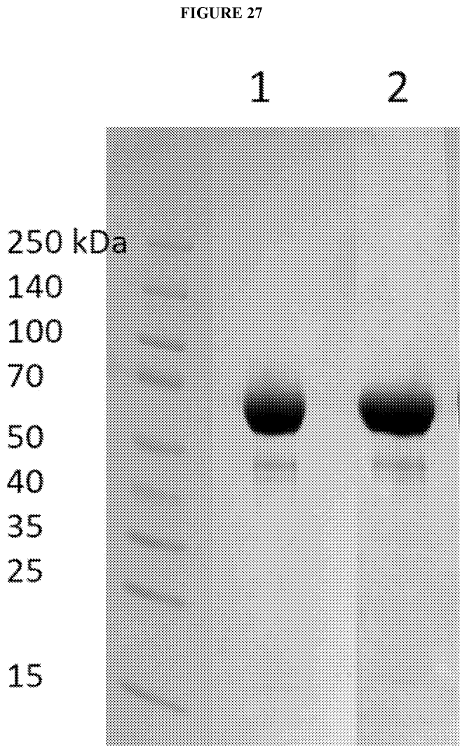

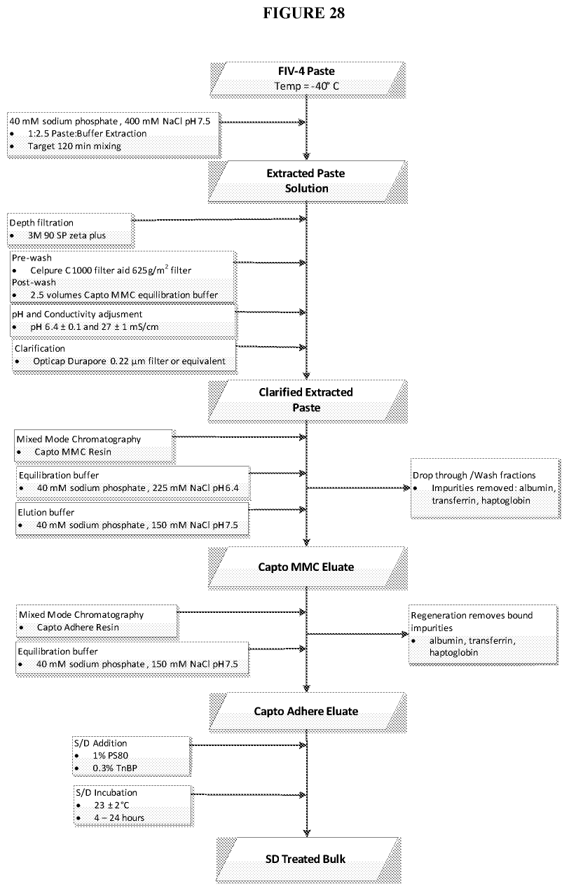

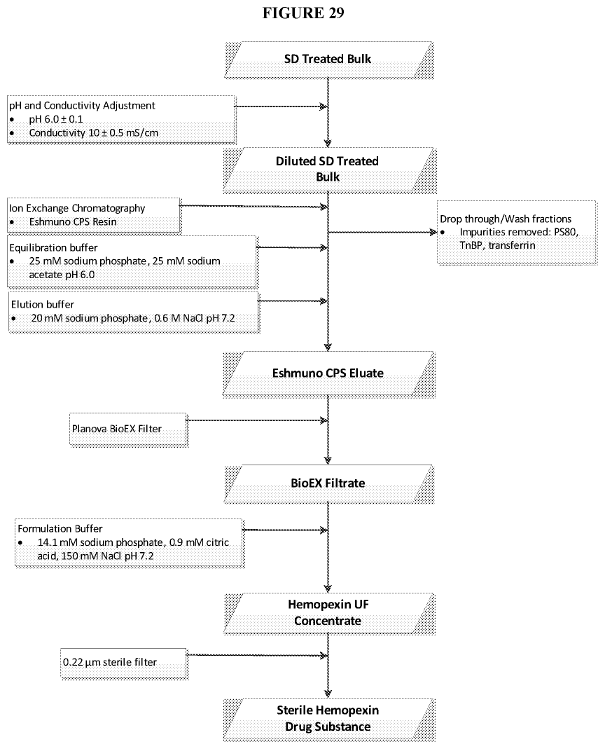

The process at pilot scale produced highly pure, active hemopexin that met or exceeded all of the quality criteria as defined by the QTPP for Phase 1 manufacture (Table 10). This is underlined by very high values for specific activity, percent monomer and purity by non-reducing SDS-PAGE (FIG. 27). The specific protein contaminants albumin, haptoglobin, transferrin and apolipoprotein A1 were reduced to extremely low levels, as were TnBP and polysorbate 80. Prekallikrein activator activity was reduced to an acceptable level, and residual protease activity was very low.

The two pilot scale batches produced highly comparable hemopexin DS, with very similar results obtained for all quality parameters. As the same batch of paste was used for both pilot scale batches, this highlights the reproducibility of the process itself.

### Example 15—Manufacturing Scale Purification Process

### Step 1: FIV-4 Paste Extraction

The hemopexin containing Fraction IV-4 (FIV-4) paste was resuspended in 40 mM sodium phosphate, 400 mM NaCl pH 7.5±0.1 (conductivity of from about 36 to about 38 mS/cm) at a 1:2.5 w/w ratio of paste: buffer by stirring for about 20 to about 120 minutes at room temperature before clarification.

### Step 2—Extracted Paste Depth Filtration and Clarification

The FIV-4 extracted paste solution was clarified by depth filtration to remove undissolved material and particulates and then further clarified through a 0.22 μm filter. Briefly, the FIV-4 extracted paste solution was depth filtered using STAX single-use disposable cartridges with EKIP filter media. Filtration of the FIV-4 extracted paste solution was then performed at a flow rate of 6.25 L/m²/min that typically resulted in a pressure of <1.5 bar during filtration. The filter press was post-washed until protein concentration is below 0.2 g/L to a maximum of 2.0 press volumes of Capto MMC equilibration buffer (40 mM sodium phosphate, 225 mM NaCl pH 6.4; conductivity of from about 23 to about 25 mS/cm). Following depth filtration, the pH of the depth filtered extract is adjusted to pH 6.4±0.1 with 0.5 M HCl, and conductivity adjusted to 27 mS with 5 M NaCl or water for injection (WFI) as appropriate. The depth filtered extract was then further clarified using a 0.5 μm or smaller filter, such as a Durapore® 0.2 μm or similar. Following filtration, the clarified extracted solution was stored <23° C. overnight or at 2-8° C. for up to 48 hours, before proceeding to processing by Capto MMC chromatography.

Step 3—Mixed Mode Chromatography with Capto MMC Resin

The clarified extracted solution obtained from the previous step (27±2 mS/cm) was loaded onto a pre-equilibrated mixed mode Capto MMC chromatography column to a target load of 14 g hemopexin/L of resin. The hemopexin concentration of the clarified extracted solution was estimated using the OD₂₈₀ₙₘ÷13, and this value was used to calculate load volume. Following product loading, the Capto MMC column was washed with equilibration buffer (40 mM sodium phosphate, 225 mM NaCl pH 6.4; conductivity of from about 23 to about 25 mS/cm) prior to elution of the bound hemopexin. At the completion of the eluate collection, the Capto MMC column was regenerated.

The hemopexin was eluted from the column with 40 mM sodium phosphate, 150 mM NaCl, pH 7.5 (conductivity of from about 18 to about 19 mS/cm). The eluted hemopexin concentration was typically between 3 and 6 mg/mL and 70 to 85% pure. The Capto MMC eluate was stored at <23° C. overnight or at 2-8° C. for up to 48 hours, before further purification by Capto Adhere chromatography.

Step 4—Mixed Mode Chromatography with Capto Adhere Resin

The Capto MMC eluate obtained from the previous step (typically at pH 7.2 with conductivity approximately 18 to 19 mS/cm) was loaded onto a pre-equilibrated mixed mode Capto Adhere chromatography column (Cytiva) to a target load of 30 g hemopexin/L of resin. The unbound hemopexin fraction was collected along with a further 3×CV wash with equilibration buffer (40 mM sodium phosphate, 150 mM NaCl, pH 7.5, conductivity of from about 18 to about 19 mS/cm) to collect any remaining hemopexin. At completion of the wash step, the Capto Adhere column was regenerated. The eluted hemopexin concentration was typically between 1 and 3 mg/mL and >90% pure. The Capto Adhere eluate was transferred directly to solvent detergent treatment. If required, the eluate can be stored at <23° C. overnight or at 2-8° C. for up to 7 days, before solvent detergent treatment.

### Step 5—Capto Adhere Pooling and Concentration

The flow through from the Capto Adhere column (Capto Adhere effluent; conductivity of about 18 mS/cm) was concentrated to reduce volume for subsequent S/D treatment and Eshmuno CPS column load steps by tangential flow filtration through a Millipore PES 10 kDa BioMax filter that was equilibrated with Capto Adhere equilibration buffer (40 mM sodium phosphate, 150 mM NaCl pH 7.5, conductivity of from about 18 to about 19 mS/cm). The Capto Adhere concentrate, which had a hemopexin content of about 20 mg/mL, was then stored overnight at <23° C. or for up to 7 days at 2-8° C., before solvent detergent treatment.

### Step 6—Solvent Detergent Virus Inactivation

The solvent/detergent (SD) treatment step was used as a first virus reduction step of the hemopexin purification process. Briefly, a suspension of Tri-n-Butyl Phosphate (TnBP) and Polysorbate 80 (PS80) was added to the concentrated Capto Adhere effluent/eluate to a target concentration of 0.3% w/w and 1.0% w/w, respectively, in the final product solution. The product solution containing TnBP and PS80 was incubated at 21-25° C. for a period of 4 to 24 hours.

Step 7—Ion Exchange Chromatography with Eshmuno CPS Resin

The solvent/detergent treated solution obtained from the previous step (conductivity of about 18-19 mS/cm) was pH adjusted to pH 6.0±0.1 with 0.5 M acetic acid and diluted with PFW or WFI to a target conductivity of from about 8.5-9.5 mS/cm. The adjusted product was loaded onto a pre-equilibrated Eshmuno CPS ion exchange chromatography column to a target load of 40 g hemopexin/L of resin. After product loading, the column was further washed with equilibration buffer (25 mM sodium phosphate, 25 mM sodium acetate, 38 mM NaCl, pH 6.0; conductivity of from about 8.5-9.5 mS/cm) to remove SD and unbound proteins. Hemopexin was eluted with 20 mM sodium phosphate, 0.6 M NaCl at pH 7.2 (conductivity of from about 50 to about 52 mS/cm) and the column was regenerated with a 1 M NaCl wash.

Step 8—Nanofiltration for Virus Removal with Planova BioEX

This step constitutes the second virus reduction step of the hemopexin manufacturing process. Briefly, the Eshmuno CPS eluate was filtered through a 0.1 μm pre-filter in series with a Planova BioEX filter with a filter area of 1 m²/100 L Eshmuno CPS eluate.

The virus filtrate can be stored at <23° C. overnight or at 2-8° C. for up to 7 days before processing by ultrafiltration and diafiltration.

### Step 9—Concentration and Diafiltration of BioEX Filtrate

The BioEX filtrate was concentrated and buffer exchanged into formulation buffer (14.1 mM di-sodium phosphate, 0.9 mM citric acid, 150 mM NaCl, pH 7.2, conductivity of about 15.5 mS/cm) using an ultrafiltration system (Millipore Pellicon 3, Biomax (PES)) with a nominal molecular weight cut off of not more than 10 kDa, and a membrane area of 0.01 m²/L BioEX filtrate. The material was concentrated to a hemopexin protein concentration of 100 mg/mL.

### Step 10—Sterile Filtration

The hemopexin UF bulk was sterile filtered into an appropriate sterile container, using a sterile 0.22 μm sterilising grade filter, at a pressure of <3 bar. This filtration is preferably performed aseptically inside a laminar flow cabinet, using sterile tubing and connectors. The sterile hemopexin solution may be stored at 2 to 8° C., or frozen at −80° C.

### Example 16—Conductivity

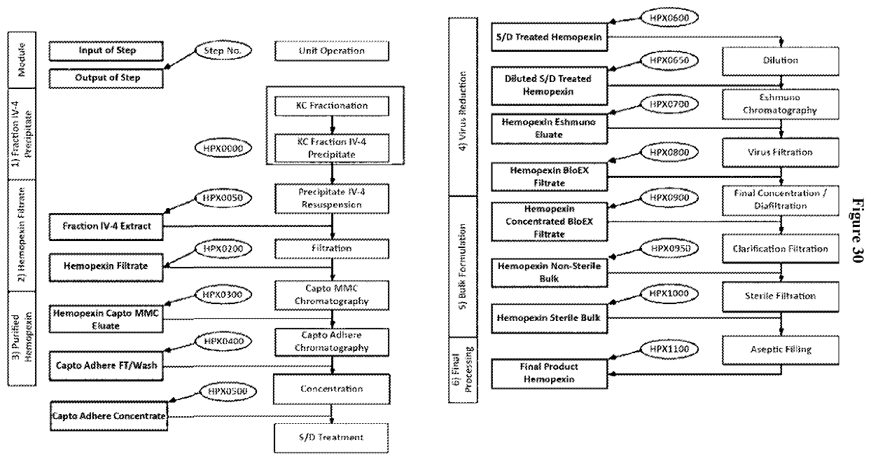

In this study, the conductivity of solutions throughout the hemopexin purification process were measured at ambient temperature (about 18° C.-23° C.) using a Thermo Fisher Orion Star A212 conductivity meter. Briefly, the purification process depicted in FIG. 30 was run at at a 1.5 kg scale, using a Fraction IV-4 plasma fraction that was filtered using a filter press prior to loading onto the Capto MMC resin. Two conductivity measurements were taken at each step, and using two different probes to allow for simultaneous readings-Probe PD (with a cell constant of 0.4750) and a recalibrated Probe PP (with a cell constant of 0.4200). Multiple conductivity readings were taken on buffers and steps requiring a conductivity adjustment.

Table 14 shows the buffer conductivity ranges and Table 15 shows the in-process conductivity ranges.

The results shows equivalent conductivity measurements using different cell constants. The recommended conductivity measurements for production were shown to be equivalent through the lab scale run.

### CONCLUSION

The strategy for development of a hemopexin purification process involved the screening of numerous candidate resins, in both high throughput and laboratory scale configurations. From these screening studies, lead candidate resins were optimised for hemopexin purity, binding capacity, recovery and operational simplicity. Viral clearance was performed with solvent/detergent treatment and by nanofiltration, with optimization of nanofiltration conditions.

Following this strategy, a method was developed for the commercially viable purification of hemopexin from Fraction IV-4 paste. The method involves three chromatographic purification steps, Capto MMC, Capto Adhere and Eshmuno CPS, and has two viral inactivation steps; solvent/detergent treatment and nano-filtration.

As the lowest binding capacity step is Capto MMC chromatography, process capacity is best expressed as a function of the size of this column, thereby making allowances for scale. The process yields approximately 8-10 grams of pure hemopexin for every litre of Capto MMC column volume, making this a high capacity process. At a column size of 250 L, this will allow processing of around 450 kg of FIV-4 paste per run.

As heme-hemopexin complex is not purified in this process, a proper estimate of process recovery requires an understanding of the amount of complex in FIV-4 paste. This can be estimated by size-exclusion chromatography, or by Capto Adhere chromatography monitored at 414 nm. When the concentration of complex is omitted, the recovery of active (non-complexed) hemopexin over the entire process was 70-72%. If recovery is calculated based on the total amount of hemopexin present (including complex), then recovery is variable, depending on the complex concentration.

During developmental studies, yields were between 0.15-0.18 grams/L PEQ. Similarly, yield relative to plasma equivalent volume varied with the amount of complex in the starting material.

The quality of hemopexin purified by this process is very high, with purity by non-reduced SDS-PAGE typically around 99%, and with monomer content also around 99%. The protein is greater than 95% active, and trace contaminants are all at very low levels.

The process was demonstrated to be quite reproducible, and was shown to be suited to large scale manufacture by the successful production of two pilot scale manufacturing batches.

On the whole, this process is well suited to commercial manufacture of high quality hemopexin, as discussed, for example, in Example 16.

TABLE 1
OD280 and concentrations by RP-HPLC
for various batches of FIV-4 paste

| | | Hemopexin | | Conc. | |
|---|---|---|---|---|---|
| Batch | | conc. | OD/conc. | estimated | % |
| no. | OD_{280 nm} | (g/L) | (HPLC) | by OD/13 | error |
| 1 | 7.10 | 0.462 | 15.4 | 0.55 | 15.4 |
| 2 | 6.32 | 0.424 | 14.9 | 0.49 | 12.8 |
| 3 | 6.85 | 0.44 | 15.4 | 0.53 | 15.7 |
| 4 | 4.16 | 0.429 | 9.7 | 0.32 | 34.1 |
| 5 | 5.01 | 0.472 | 10.6 | 0.39 | 22.5 |
| 6 | 6.29 | 0.445 | 14.1 | 0.48 | 8.0 |
| 7 | 6.69 | 0.506 | 13.2 | 0.51 | 1.7 |
| 8 | 6.96 | 0.465 | 15.0 | 0.54 | 13.1 |
| 9 | 6.58 | 0.534 | 12.3 | 0.51 | 5.5 |
| 10 | 5.07 | 0.433 | 11.7 | 0.39 | 11.0 |
| Mean | 6.103 | 0.461 | 13.2 | 0.47 | 13.98 |

TABLE 2
Hemopexin quality in Capto MMC eluate after storage

| | | T = 0 | Storage at 4 degrees | | | Storage at RT | | |
|---|---|---|---|---|---|---|---|---|
| | | Freeze | T = 0 | T = 2 | T = 7 | T = 0 | T = 2 | T = 7 |
| Property | T = 0 | thaw | control | days | days | control | days | days |
| Conc by | 6.97 | 7.21 | 6.99 | 6.99 | 7.08 | 6.99 | 7.03 | 7.14 |
| Nephelometry | | | | | | | | |
| (mg/mL ) | | | | | | | | |
| Purity (SDS page) | 91.20% | 91.00% | | No | No | | No | No |
| | | | | difference | difference | | difference | difference |
| | | | | with | with | | with | with |
| | | | | t = 0 | t = 0 | | t = 0 | t = 0 |
| Monomer content by | 98.00% | 97.20% | 98.13% | 97.95% | | 98.13% | 97.96% | |
| SEC-HPLC | | | 97.99% | | 97.98% | 97.99% | | 97.67% |
| Haem binding activity | 6.10 | 6.16 | 6.21 | 6.14 | | 6.21 | 6.18 | |
| | | | 6.00 | | 5.96 | 6.00 | | 6.02 |
| Charge Heterogeneity | 8 | No | No | No | No | No | No | No |
| by cIEF | Major | difference | difference | difference | difference | difference | difference | difference |
| | species pI | with | with | with | with | with | with | with |
| | 5.6-6.1 | t = 0 | t = 0 | t = 0 | t = 0 | t = 0 | t = 0 | t = 0 |

TABLE 3
Yield, recovery and quality data for hemopexin purified
from several batches at small laboratory scale

| | 2 | 10 | 1 |
|---|---|---|---|
| Yield (g/LPEQ; neph) | 0.12 | 0.15 | 0.15 |
| Recovery (%; neph) | 54 | 61 | 57 |
| Purity (%; SDS-PAGE) | 99.9 | 99.8 | 99.7 |
| Specific Activity (%)* | 104.9 | 100.5 | 99.6 |

*Specific activity - amount of hemopexin able to bind heme/amount total protein.

TABLE 4
Characterisation results for the hemopexin drug
substance from initial laboratory scale batches

| Assay | Units | Batch 1 | Batch 2 |
|---|---|---|---|
| Hemopexin Concentration | g/L | 126 | 45.2 |
| Heme Binding | mg/mL | 99.1 | 37.1 |
| Heme Specific Activity | % | 91 | 92 |
| Transferrin Concentration | g/L | <0.0878 | <0.0878 |
| Albumin Concentration | g/L | <0.34 | <0.34 |
| Haptoglobin Concentration | g/L | <0.075 | <0.075 |
| Purity (NR SDS-PAGE) | % | 96 | 90 |
| SE-HPLC | | | |
| HMW Aggregates | % | 1.4 | 4.4 |
| Monomer | % | 98.2 | 95.3 |
| LMW Proteins | % | 0.4 | 0.3 |

TABLE 5
Changed process conditions for optimised laboratory scale batch

| | Initial Laboratory | |
|---|---|---|
| | Scale Batch | Optimised Laboratory |
| Process Step | Conditions | Scale Batch Conditions |
| FIV-4 Paste Extraction | 40 mM phosphate, 250 | 40 mM phosphate, 225 |
| Extraction Buffer | mM NaCl pH 6.5 | mM NaCl pH 7.5 |
| Depth Filtration & | 2.5 cm | 4 cm |
| Clarification | | |
| Filter Frame Depth | | |
| Capto MMC | 40 mM phosphate, 250 | 40 mM phosphate, 225 |
| Chromatography | mM NaCl pH 6.5 | mM NaCl pH 6.4 |
| Equilibration Buffer | | |
| Capto Q resin replaced | Capto Q | Eshmuno CPS |
| with Eshmuno CPS | 10 mM phosphate pH | 25 mM sodium acetate, |
| resin | 7.5 | 25 mM sodium |
| Equilibration buffer | | phosphate, 38 mM NaCl |
| | | pH 6.0 |
| Elution buffer | Formulation buffer | 20 mM phosphate, 600 |
| | | mM NaCl pH 7.2 |

TABLE 6
Characterisation results for the hemopexin drug
substance from optimised laboratory scale batches

| Assay | Units | Optimised Batch |
|---|---|---|
| Hemopexin Concentration | g/L | 92.0 |
| Heme Binding | mg/mL | 89.6 |
| Heme Specific Activity | % | 97 |
| Transferrin Concentration | g/L | <0.00878 |
| Albumin Concentration | g/L | <0.0085 |
| Haptoglobin Concentration | g/L | <0.0075 |
| Purity (NR SDS-PAGE) | % | >99% |
| SE-HPLC | | |
| HMW Aggregates | % | 0.4 |
| Monomer | % | 99.5 |
| LMW Proteins | % | 0.1 |
| Yield | g/LPEQ | 0.14 |
| Recovery | % | 73 |

TABLE 7
Changed process conditions for streamlined laboratory scale batch

| | Optimised Laboratory | Streamlined Laboratory |
|---|---|---|
| Process Step | Scale Batch Conditions | Scale Batch Conditions |
| FIV-4 Paste | 40 mM phosphate, 225 | 40 mM phosphate, 400 |
| Extraction | mM NaCl pH 7.5 | mM NaCl pH 7.5 |
| Extraction Buffer | | |
| Adjustment of | Adjust pH to 6.4 | Adjust pH to 6.4 |
| Depth Filtrate | Adjust conductivity to | |
| | 27-28 mS/cm | |
| Capto Adhere | Adjust pH to 7.5 | No adjustment |
| Chromatography | Adjust conductivity to 20 | |
| Adjustment of | mS/cm | |
| Capto MMC Eluate | | |
| Eshmuno CPS | 20 mM phosphate, 600 | 20 mM phosphate, 150 |
| Chromatography | mM NaCl pH 7.2 | mM NaCl pH 7.2 |
| Elution Buffer | | |

TABLE 8
Protease activity in intermediate fractions from pilot scale batches

| Process Stage | Batch A (nkat/L) | Batch B (nkat/L) |
|---|---|---|
| Extracted Paste | 399.8 | 30.4 |
| Clarified Extracted Paste | <3 | <3 |
| Capto MMC Eluate | <3 | <3 |
| Capto Adhere Eluate | <3 | <3 |
| Eshmuno CPS Eluate | <3 | <3 |
| BioEx filtrate | N/D | N/D |
| Hemopexin DS | <3 | <3 |

TABLE 9
Prekallikrein activator in intermediate
fractions from pilot scale batches

| Process Stage | Batch A (IU/mL) | Batch B (IU/mL) |
|---|---|---|
| Clarified Extracted Paste | N/D | <20 |
| Capto MMC Eluate | <20 | <20 |
| Hemopexin DS | <20 | <20 |

TABLE 10
Characterisation results for the sterile hemopexin drug substance

| | | Phase 1 | | |
|---|---|---|---|---|
| | | QTPP | | |
| Assay | Units | Criteria | Batch A | Batch B |
| Hemopexin | g/L | — | 103.7 | 106.4 |
| Concentration | | | | |
| Heme Binding | μM | 1330-1897 | 1708 | 1703 |
| Heme Specific | % | ≥85 | 100 | 97 |
| Activity | | | | |
| CD91 Binding KD | μM | — | 1.13 ± 0.06 | 1.06 ± 0.05 |
| Transferrin | g/L | — | 0.115 | 0.206 |
| Concentration | | | | |
| Albumin | g/L | — | <0.0085 | <0.0085 |
| Concentration | | | | |
| Haptoglobin | g/L | — | 0.0276 | 0.0097 |
| Concentration | | | | |
| Apo-A1 Concentration | g/L | — | <0.0497 | <0.0497 |
| SE-HPLC | | | | |
| HMW Aggregates | % | | 0.5 | 0.4 |
| Monomer | % | ≥94 | 99.2 | 99.3 |
| LMW Proteins | % | | 0.3 | 0.3 |
| Purity by Reduced | % | ≥84 | 94 | 89 |
| SDS-PAGE | | | | |
| Purity by Non-reduced | % | — | 99 | 99 |
| SDS-PAGE | | | | |
| iCEF (BMW Method) | | | | |
| Minimum pI | | — | 5.51 | 5.53 |
| Main pI | | | 5.83 | 5.85 |
| Maximum pI | | | 6.03 | 6.04 |
| Protease Activity | nKat/L | — | ≤3 | ≤3 |
| Prekallikrein Activity | IU/mL | <20 | <20 | <20 |
| TnBP Concentration | μg/mL | <2 | <5 | <5 |
| (QB6694) | | | | |
| PS80 Concentration | mg/L | <100 | <8 | 16 |
| (QB7751) | | | | |

TABLE 11
Step recovery of hemopexin for pilot scale batches as
measured by RP-HPLC

| | Step recovery (%) | | | | | |
|---|---|---|---|---|---|---|
| | Depth | | | | Virus | UF/DF & |
| | Filtration & | Capto | Capto | Eshmuno | Fil- | Sterile |
| | Clarification | MMC | Adhere | CPS | tration | Filtration |
| Batch A | 80 | 87 | 88 | 100 | 98 | 101 |
| Batch B | 83 | 84 | 59 | 130 | 105 | 102 |

TABLE 12
Step recovery of transferrin, albumin and haptoglobin for
pilot scale batches

| | Step recovery (%) | | | | | | | |
|---|---|---|---|---|---|---|---|---|
| | Depth Filtration & | | Capto | | Capto | | Eshmuno | |
| | Clarification | | MMC | | Adhere | | CPS | |
| | Batch | Batch | Batch | Batch | Batch | Batch | Batch | Batch |
| | A | B | A | B | A | B | A | B |
| T | 86 | 91 | 1.1 | 1.4 | 16 | 12 | 13 | 26 |
| A | 79 | 69 | 0.3 | 0.3 | | | | |
| H | 78 | 61 | 0.6 | 0.9 | | | | |

T = transferrin;
A = albumin;
H = haptoglobin.

TABLE 13
Process recovery for pilot scale batches

| | Recovery (%) | | | |
|---|---|---|---|---|
| | | | Hemopexin | Hemopexin |
| | | | without | without |
| | Hemopexin | Hemopexin | complex | complex |
| | (Neph) | (RP-HPLC) | (Neph) | (RP-HPLC) |
| Batch A | 59 | 54 | 74 | 67 |
| Batch B | 57 | 53 | 71 | 66 |

TABLE 14
Processing Conductivity Measurements
Buffer Conductivity Measurements

| | PD probe | PP probe |
|---|---|---|
| HPX Buffer | (mS/cm) | (mS/cm) |
| Extraction Buffer | 41-43 | 36.4-38.2 |
| MMC Equilibration Buffer | 26-28 | 23-25 |
| MMC Elution/Adhere Equilibration | 20-21 | 18-18.7 |
| Buffer | | |
| Regeneration Buffer | 70+ | 70+ |
| Eshmuno Equilibration Buffer | 9.5-10.5 | 8.5-9.4 |
| Eshmuno Elution Buffer | 57-58 | 50.5-51.4 |
| Virus Wash Buffer | 35-39 | 31-34.7 |
| Final Formulation Buffer* | 17.4 | 15.5 |

*Indicates no range provided, expected reading provided

TABLE 15
Processing Conductivity Measurements
Processing Conductivity Measurements

| | PD probe | PP probe |
|---|---|---|
| HPX Processing step | (mS/cm) | (mS/cm) |
| HPX0200- Filtrate | 26-28 | 23-25 |
| HPX0300- MMC Eluate | 20-21 | 17.8-18.6 |
| HPX0400- Adhere FT/Wash* | 20 | 18 |
| HPX0500- Adhere Concentrate* | 19.5 | 17.3 |
| HPX0650- Eshmuno Load | 9.5-10.5 | 8.5-9.3 |
| HPX0700- Eshmuno Eluate* | 44.5 | 40 |
| HPX0900- Final UF/DF | 13.3 | 11.7 |
| Concentrate* | | |

*Indicates not an adjustment step, expected reading provided

1. A method of purifying hemopexin from a solution containing hemopexin and other proteins, the method comprising:
(i) providing a solution comprising hemopexin and other proteins, wherein the solution comprises less than about 300 mM sodium chloride (NaCl);
(ii) passing the solution of step (i) through a mixed-mode cation exchange chromatography resin under conditions that promote selective binding of hemopexin to the resin over binding of the other proteins to the resin;
(iii) washing the resin after step (ii) to remove unbound proteins;
(iv) eluting the hemopexin bound to the resin after step (iii); and
(v) recovering the hemopexin eluted in step (iv).

2. The method of claim 1, wherein the mixed-mode cation exchange chromatography resin the structure of formula (I) or (II):

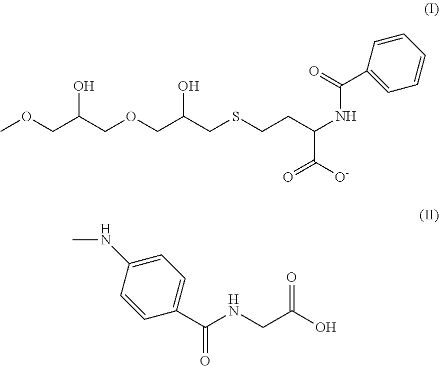 [SMILES: COCC(O)COCC(O)CSCCC(NC(=O)c1ccccc1)C(=O)[O-] ; CNc1ccc(C(=O)NCC(=O)O)cc1]

3. The method of claim 1 or claim 2, wherein the recovered hemopexin eluate of step (v) has a purity of at least about 50%.

4. The method of claim 3, wherein the recovered hemopexin eluate of step (v) has a purity of from about 70% to about 99%.

5. The method of any one of claims 1 to 4, wherein the solution of step (i) has a pH of from about 6.2 to about 6.6.

6. The method of claim 5, wherein the solution of step (i) has a pH of about 6.4.

7. The method of any one of claims 1 to 6, wherein the solution of step (i) comprises from about 160 mM to about 250 mM NaCl.

8. The method of claim 7, wherein the solution of step (i) comprises from about 200 mM to about 250 mM NaCl.

9. The method of claim 8, wherein the solution of step (i) comprises about 225 mM NaCl.

10. The method of any one of claims 1 to 9, wherein the solution of step (i) comprises:
(a) a pH of from about 6.2 to about 6.6;
(b) from about 20 mM to about 60 mM phosphate buffer; and
(c) from about 160 mM to about 250 mM NaCl.

11. The method of claim 10, wherein the solution of step (i) comprises:
(a) a pH of about 6.4;
(b) about 40 mM phosphate buffer; and
(c) about 225 mM NaCl.

12. The method of any one of claims 1 to 11, wherein the amount of hemopexin that is passed through the resin in step (ii) is from about 1 mg to about 40 mg per mL of resin.

13. The method of any one of claims 1 to 12, wherein the solution of step (i) has a conductivity of from about 23 mS/cm to about 28 mS/cm.

14. The method of claim 13, wherein the solution of step (i) has a conductivity of from about 23 mS/cm to about 25 mS/cm.

15. The method of any one of claims 1 to 14, wherein the solution is a human plasma fraction.

16. The method of claim 14, wherein the solution of step (i) is derived from a Cohn Fraction IV.

17. The method of claim 16, wherein the solution of step (i) is derived from a Cohn Fraction IV₄.

18. The method of claim 16 or claim 17, wherein the solution of step (i) is prepared by (a) resuspending the Cohn Fraction IV in an extraction buffer to obtain a resuspended Cohn Fraction IV, (b) passing the resuspended Cohn Fraction IV of step (a) through a filter, and (c) recovering the filtered Cohn Fraction IV extract from step (b).

19. The method of claim 18, wherein step (a) comprises resuspending the Cohn Fraction IV in an extraction buffer at a Cohn Fraction IV: extraction buffer ratio of from about 1:2 to about 1:20.

20. The method of claim 19, wherein step (a) comprises resuspending the Cohn Fraction IV in an extraction buffer at a Cohn Fraction IV: extraction buffer ratio of about 1:2.5.

21. The method of any one of claims 18 to 20, wherein the extraction buffer has a pH of from about 6 to about 8.

22. The method of claim 21, wherein the extraction buffer has a pH of from about 6.2 to about 7.5.

23. The method of claim 22, wherein the extraction buffer has a pH of about 7.5.

24. The method of any one of claims 18 to 23, wherein the extraction buffer comprises from about 20 mM to about 500 mM NaCl.

25. The method of claim 24, wherein the extraction buffer comprises about 400 mM NaCl.

26. The method of claim 25, wherein the extraction buffer comprises about 40 mM sodium phosphate and about 400 mM NaCl.

27. The method of any one of claims 18 to 26, wherein the filtered Cohn Fraction IV extract of step (c) is passed through a fine filter having a pore size of about 0.5 μm or less to obtain a clarified Cohn Fraction IV extract.

28. The method of claim 27, wherein the pH of the clarified Cohn Fraction IV extract is adjusted to a value of from about 6.2 to about 6.6.

29. The method of claim 28, wherein the pH of the clarified Cohn Fraction IV extract is adjusted to about 6.4.

30. The method of any one of claims 27 to 29, wherein the conductivity of the clarified Cohn Fraction IV extract is adjusted to a value of from about 24 mS/cm to about 30 mS/cm.

31. The method of claim 30, wherein the conductivity of the clarified Cohn Fraction IV extract is adjusted to a value of from about 26 mS/cm to about 28 mS/cm.

32. The method of any one of claims 1 to 31, wherein the conductivity of the recovered hemopexin eluate of step (v) is from about 18 to about 19 mS/cm.

33. The method of any one of claims 1 to 32, further comprising:
(vi) passing the recovered hemopexin eluate of step (v) through a mixed-mode anion exchange chromatography resin under conditions that allow any impurities in the recovered hemopexin eluate to bind to the resin while allowing the hemopexin to pass through the resin as an unbound fraction; and
(vii) recovering the unbound fraction comprising hemopexin.

34. The method of claim 33, wherein the mixed-mode anion exchange chromatography resin from step (vi) is equilibrated with a buffer comprising a pH of from about 7.0 to about 8.0.

35. The method of claim 33 or claim 34, wherein the equilibration buffer has a pH of about 7.5.

36. The method of any one of claims 33 to 35, wherein the equilibration buffer comprises from about 100 mM to about 200 mM NaCl.

37. The method of claim 36, wherein the equilibration buffer comprises about 150 mM NaCl.

38. The method of any one of claims 33 to 37, further comprising exposing the recovered unbound fraction of step (vii) to a virus inactivation step to obtain a virus inactivated hemopexin solution.

39. The method of claim 38, wherein the virus inactivation step comprises exposing the recovered unbound fraction of step (vii) to a solution comprising a surfactant and a solvent.

40. The method of claim 39, wherein the solvent is tri-n-butyl phosphate (TnBP).

41. The method of claim 39 or claim 40, wherein the surfactant is polysorbate 80 (PS80).

42. The method of claim 40 or claim 41, wherein the solvent detergent treatment comprises exposing the recovered unbound fraction of step (vii) to 1% polysorbate 80 (PS80) and 0.3% tri-n-butyl phosphate (TnBP).

43. The method of any one of claims 38 to 42, further comprising:
(ix) passing the virus inactivated hemopexin solution through an ion exchange chromatography resin under conditions that allow the hemopexin to bind to the resin;
(x) optionally washing the resin following step (ix); and
(xi) eluting the hemopexin bound to the resin in step (ix); and
(xii) recovering the eluted hemopexin from step (xi).

44. The method of claim 43, wherein the ion exchange chromatography resin is a cation exchange chromatography resin or an anion exchange chromatography resin.

45. The method of claim 43 or claim 44, wherein prior to step (ix), the pH of the virus inactivated hemopexin solution is adjusted to a value of from about 6.0 to about 6.2.

46. The method of claim 45, wherein prior to step (ix), the pH of the virus inactivated hemopexin solution is adjusted to about 6.0.

47. The method of any one of claims 43 to 46, wherein prior to step (ix), the conductivity of the virus inactivated hemopexin solution is adjusted to a value of from about 8 mS/cm to about 12 mS/cm.

48. The method of any one of claims 43 to 46, wherein prior to step (ix), the conductivity of the virus inactivated hemopexin solution is adjusted to about 10 mS/cm.

49. The method of any one of claims 43 to 48, further comprising exposing the eluted hemopexin recovered in step (xii) to diafiltration to adjust the concentration of the eluted hemopexin to a value of from about 50 mg/mL to about 120 mg/mL.

50. The method of claim 49, wherein the concentration of the eluted hemopexin is adjusted to about 100 mg/mL.

51. The method of any one of claims 1 to 50, wherein the recovered hemopexin is subjected to virus filtration.

52. The method of claim 51, wherein the virus filtration comprises passing the recovered hemopexin through a virus filter having a pore size of from about 15 nm to about 20 nm diameter.

53. A composition comprising the hemopexin recovered by the method of any one of claims 1 to 52.

54. A composition comprising a combination of any two or more of the characteristics selected from the group consisting of:
(a) a hemopexin content of from about 95 mg/mL to about 110 mg/mL;
(b) heme binding activity of from about 1000 μM to about 2000 μM;
(c) heme specific binding activity of at least about 80% of total protein
(d) a CD91 dissociation constant (KD) of from about 0.50 μM to about 2.0 μM;
(e) a transferrin content of less than about 0.50 mg/mL;
(f) an albumin content of less than about 0.05 mg/mL;
(g) a haptoglobin content of less than about 0.05 mg/ml;
(h) an Apo-A1 content of less than about 0.10 mg/mL;
(i) a high molecular weight (HMW) hemopexin aggregate content of less than about 1.0% of total protein, as determined by size exclusion-high-performance liquid chromatography;
(j) a hemopexin monomer content of at least about 90% of total protein, as determined by size exclusion-high-performance liquid chromatography;
(k) a low molecular weight (LMW) impurity content of less than about 1.0% of total protein, as determined by size exclusion-high-performance liquid chromatography;
(l) a hemopexin purity content of at least about 80% of total protein, as determined by reduced SDS-PAGE or as determined by non-reduced SDS-PAGE;
(m) an isoelectric point (pI) of from about 5.0 to about 6.5, as determined by Capillary isoelectric focusing (cIEF);
(n) a level of protease activity of less than about 5 nKat/L;
(o) a level of prekallikrein activity of less than about 30 IU/mL;
(p) a tri (n-butyl) phosphate (TnBP) content of less than about 10 μg/mL; and
(q) a PS80 content of less than about 20 mg/mL.

55. A composition comprising a combination of any two or more of the characteristics selected from the group consisting of:
(a) a hemopexin content of from about 95 mg/mL to about 110 mg/mL;
(b) heme binding activity of from about 1600 μM to about 1800 μM;
(c) heme specific binding activity of at least about 97% of total protein;
(d) a CD91 dissociation constant (KD) of from about 1.10 μM to about 1.20 μM;
(e) a transferrin content of less than about 0.25 mg/ml;
(f) an albumin content of less than about 0.009 mg/mL;
(g) a haptoglobin content of less than about 0.03 mg/ml;
(h) an Apo-A1 content of less than about 0.06 mg/mL;
(i) a high molecular weight (HMW) hemopexin aggregate content of less than about 0.6% of total protein;
(j) a hemopexin monomer content of at least about 99% of total protein;
(k) a low molecular weight (LMW) impurity content of less than about 0.4% of total protein;
(l) a hemopexin purity content of at least about 88% of total protein;
(m) an isoelectric point (pI) of from about 5.4 to about 6.3;
(n) a level of protease activity of less than about 3 nKat/L;
(o) a level of prekallikrein activity of less than about 20 IU/mL;
(p) a tri (n-butyl) phosphate (TnBP) content of less than about 5 μg/mL; and
(q) a PS80 content of less than about 18 mg/mL.

56. A composition comprising (i) a hemopexin content of from about 95 mg/mL to about 110 mg/mL, (ii) a hemopexin monomer content of at least about 99% of total protein, and (ii) a heme specific binding activity of at least about 97% of total protein.

57. The composition of claim 56, further comprising (i) a transferrin content of less than about 0.25 mg/mL and (ii) a haptoglobin content of less than about 0.03 g/L haptoglobin.

58. The composition of claim 56 or claim 57, further comprising no detectable apolipoprtein A1 and/or albumin content.

59. The composition of claim 56 or claim 57, further comprising (i) an albumin content of less than about 0.009 mg/mL, and (ii) an Apo-A1 content of less than about 0.06 mg/mL.

60. The composition of any one of claims 56 to 59, further comprising comprises no detectable protease activity.

61. A formulation comprising the composition of any one of claims 53 to 60 and a pharmaceutically acceptable carrier.

62. The formulation of claim 61, comprising about 15 mM citrate phosphate buffer, about 150 mM NaCl, a pH of about 7.2 and a concentration of hemopexin of about 100 mg/mL.

63. The composition of any one of claims 53 to 60 or the formulation of claim 61 or claim 62, wherein the composition or formulation is suitable for pharmaceutical administration after storage for 12 months at 2° C. to 8° C. and/or at ambient temperature.

64. A method of treating a condition associated with hemolysis, the method comprising administering to a subject in need thereof the composition of any one of claims 53 to 60 and 63, or the formulation of any one of claims 61 to 63.

65. The method of claim 64, wherein the condition is an acute hemolytic condition or a chronic hemolytic condition.

66. The method of claim 65, wherein the condition is selected from the group consisting of hemolytic anaemia, transfusion-induced hemolysis, hemolytic uraemic syndrome, an autoimmune disease, malaria infection, trauma, blood transfusion, open heart surgery using cardiopulmonary bypass and burns, hemoglobinemia and hemoglobinuria accompanied with hemolysis after burn.

67. The method of claim 65, wherein the condition is selected from the group consisting of sickle cell anaemia, hereditary spherocytosis, hereditary elliptocytosis, thalassemia, congenital dyserythropoietic anemia and Paroxysmal nocturnal hemoglobinuria, systemic lupus erythematosus and chronic lymphocytic leukemia.

68. Use of the composition of any one of claims 53 to 60 and 63 in the manufacture of a medicament for treating a condition associated with hemolysis.

69. The use of claim 68, wherein the condition is selected from the group consisting of hemolytic anaemia, transfusion-induced hemolysis, hemolytic uraemic syndrome, an autoimmune disease, malaria infection, trauma, blood transfusion, open heart surgery using cardiopulmonary bypass and burns, hemoglobinemia and hemoglobinuria accompanied with hemolysis after burn.

70. The use of claim 68, wherein the condition is selected from the group consisting of sickle cell anaemia, hereditary spherocytosis, hereditary elliptocytosis, thalassemia, congenital dyserythropoietic anemia and Paroxysmal nocturnal hemoglobinuria, systemic lupus erythematosus and chronic lymphocytic leukemia.

71. The composition of any one of claims 53 to 60 and 63 or the formulation of any one of claims 61 to 63 for use in treating a condition associated with hemolysis.

72. The composition for use according to claim 71, wherein the condition is selected from the group consisting of hemolytic anaemia, transfusion-induced hemolysis, hemolytic uraemic syndrome, an autoimmune disease, malaria infection, trauma, blood transfusion, open heart surgery using cardiopulmonary bypass and burns, hemoglobinemia and hemoglobinuria accompanied with hemolysis after burn.

73. The composition for use according to claim 71, wherein the condition is selected from the group consisting of sickle cell anaemia, hereditary spherocytosis, hereditary elliptocytosis, thalassemia, congenital dyserythropoietic anemia and Paroxysmal nocturnal hemoglobinuria, systemic lupus erythematosus and chronic lymphocytic leukemia.
